(19) United States
(12) Patent Application Publication
(10) Pub. No.: US 2003/0060663 A1
(43) Pub. Date: Mar. 27, 2003

(54) Novel therapeutic agents for macromolecular structures

(76) Inventors: John H. Griffin, Atherton, CA (US); Burton G. Christensen, Alamo, CA (US); Thomas E. Jenkins, La Honda, CA (US); J. Kevin Judice, El Granada, CA (US)

Correspondence Address: ADVANCED MEDICINE, INC., 901 GATEWAY BOULEVARD, SOUTH SAN FRANCISCO, CA, 94080, US

(21) Appl. No.: 10/096,270

(22) Filed: Mar. 8, 2002

Related U.S. Application Data
(63) Continuation of application No. 09/510,176, filed on Feb. 22, 2000.
Application No. 09/510,176 is a continuation of application No. 09/326,660, filed on Jun. 7, 1999, now abandoned.
(60) Provisional application No. 60/088,464, filed on Jun. 8, 1998.
(60) Provisional application No. 60/092,941, filed on Jul. 15, 1998.

(51) Int. Cl. C07C 233/57

(52) U.S. Cl. 564/152; 564/123

(57) ABSTRACT

Disclosed are novel multibinding compounds (agents) which bind macromolecular structures including cellular, extracellular, and microbial components derived from vectors, viruses, fungi, yeasts, bacteria, and the like. The compounds of this invention comprise a plurality of ligands each of which can bind to such macromolecular structures thereby modulating the biological processes/functions thereof. Each of the ligands is covalently attached to a linker (framework) to provide for the multibinding compound. The linker is selected such that the multibinding compound so constructed demonstrates increased modulation or disruption of the biological processes/functions of cell as compared to the aggregate of the individual units of the ligand.

### CROSS-REFERENCE TO RELATED APPLICATIONS

This application claims the benefit of the following U.S. Provisional Patent Applications:

1. U.S. Provisional Application No. 60/088,464, filed Jun. 8, 1998; and

2. U.S. Provisional Application No. 60/092,941, filed Jul. 15, 1998; each of these applications are incorporated herein by reference in its entirety.

### BACKGROUND OF THE INVENTION

1. Field of the Invention

This invention relates to novel multibinding compounds (agents) which bind macromolecular structures including cellular and extracellular components of mammalian systems along with microbial components derived from vectors, viruses, fungi, parasites, yeasts, bacteria, and the like. The compounds of this invention comprise a plurality of ligands each of which can bind macromolecular structures thereby modulating the biological processes/functions thereof. Each of the plurality of ligands is covalently attached to a linker (framework) to provide for the multibinding compound. The linker is selected such that the multibinding compound so constructed demonstrates increased modulation or disruption of the biological processes/functions of cells (as defined herein) as compared to the aggregate of the individual units of the ligand made available for binding to the cells.

These multibinding compounds are particularly useful in treating pathologic conditions and other conditions which benefit from therapeutic treatment which are mediated by cells comprising macromolecular structures targeted by the ligands. Accordingly, this invention also relates to pharmaceutical compositions comprising a pharmaceutically acceptable excipient and an effective amount of a compound of this invention.

Still further, this invention is directed to methods for treating pathologic conditions in mammals which are mediated by cells comprising macromolecular structures targeted by a ligand which method comprises administering to such a mammal an effective amount of a pharmaceutical composition of this invention.

This invention also provides for combinatorial synthetic methods for providing libraries of multimeric compounds useful in identifying compounds exhibiting properties consistent with multibinding compounds. Libraries of such compounds are also provided as well as iterative methods for identifying multibinding compounds targeting macromolecular receptors.

References

The following publications are cited in this application as superscript numbers:

¹ Carrasco, Pharmac. Ther., 64:215-290 (1994)

² Diana, et al., J. Med. Chem., 36:3240-3250 (1993)

³ Rossman, Protein Sci., 3:1712-1725 (1994)

⁴ Diana, et al., J. Med. Chem., 38:1355-1371 (1995)

⁵ Diana, et al., J. Med. Chem., 37:2421-2436 (1995)

⁶ Rychnovsky, et al., J. Am. Chem. Soc., 117:197(1995)

⁷ Rychnovsky, et al., Angew. Chem. Intl. Ed., 34:1227 (1997)

⁸ Warnock, J. Antimicrob. Chemother., 28:27-38 (1991)

⁹ Witzke, et al., Biochemistry, 23:1668-1674 (1984)

¹⁰ Brajtburg, et al., Antimicrob. Agents, Chemother., 34:183-188 (1990)

¹¹ Kleinberg, et al., J. Membrane Biol., 80:257-269 (1984)

¹² Medoff, et al., Antifungal Chemotherapy, John Wiley & Sons, Publisher, New York, N.Y., pp. 3-33 (1980)

¹³ Bolard, Biochim. Biophys. Acta, 864:257-304 (1986)

¹⁴ Bennet in Goodman & Gilman's The Pharmacological Basis of Therapeutics, 9th Ed. (Hardman, et al., Eds.); McGraw-Hill, New York, N.Y., pp.1175-1190 (1996)

¹⁵ Sawaya, et al., J. Am. Soc. Nephrol., 6:154-164 (1995)

¹⁶ Hartsel, et al., Trends Pharmacol. Sci., 17:445-449 (1996)

¹⁷ Rapp, et al., Ann. Pharmacother, 31:1174-1186 (1997)

¹⁸ Yamashita, et al., J. Am. Chem. Soc., 117:6249-6253 (1995)

¹⁹ Athar, et al., J. Med. Microbiol., 4:505-517 (1971)

²⁰ Pierce, et al., Can. J. Biochem., 56:135-142 (1978)

²¹ Gold, et al., Antibiotic Ann., 579:1955-1956

²² Nicolau, et al., J. Am. Chem. Soc., 110:4660-4672 (1988)

²³ Kennedy, et al., Tetrahedron Lett., 29:447-450 (1988)

²⁴ Nicolau, et al., J. Am. Chem. Soc., 110:4696-4705 (1988)

²⁵ Hammond, Prog. Med. Chem., 14:105-179 (1977)

²⁶ Czerwinski, et al., J. Antibiotics, 43:1098-1100 (1990)

²⁷ Cheron, et al., Biochem. Pharmacol., 37:827-836 (1978)

²⁸ Czerwinski, et al., J. Antibiotics, 43:680-683 (1990)

²⁹ Grzybowska, et al., J. Antibiotics, 43:907-908 (1990)

³⁰ Hoeprich, et al., Ann. NY Acad. Sci., 544:517-546 (1988)

³¹ Parmegiani, et al., Antimicrob. Agents Chemother., 31:1756-1760 (1987)

³² Czerwinski, et al., J. Antibiotics, 44:979-984 (1991)

³³ Wright, et al., J. Antibiotics, 35:911-914 (1982)

³⁴ Taylor, et al., J. Antibiotics, 46:486-493 (1993)

³⁵ Alberts, et al., Molecular Biology of the Cell, Second Edtion, Garland Publishing, New York, Chapters 11 and 13 (1989).

³⁶ Recommendations for the Safe Handling of Perenteral Antineoplastic Drugs, NIH Publication No. 83-2621. For sale by the Superintendent of Documents, US Government Printing Office, Washington, D.C. 20402]

³⁷ Klopman, G., et al., Molecular Pharmacology 52:323-334 (1997)

³⁸ Green, et al., Arch. Dermatol., 124:1718, November 1988

³⁹ Tang-Wai, D. F., et al., Biochemistry, 32:6470-6476 (1993)

⁴⁰ Li, L., et al., J. Med. Chem., 37:3400-3407 (1994)

⁴¹ Chen, K., et al., J. Med. Chem., 40:3049-3056 (1997)

⁴² Petit, G. R., et al., J. Med. Chem., 38:1666-1672 (1995)].

⁴³ Lee, K. H., “Antineoplastic agents from plants; Kingdom, A. D., Balandrin, M., Eds.; American Chemical Society Symposium Series 534; American Chemical Society: Washington, D.C., Chapter 12, pp. 170-190 (1997)

⁴⁴ Hamel, E., et al., Biochem. Pharmacol., 37:2445-2449 (1988)

⁴⁵ Wang, R. W. J., et al., Cancer Res., 37:3071-3079 (1977)

⁴⁶ Ye, K., et al., Proc. Natl. Acad. USA, 95:1601-1606 (1998)

⁴⁷ IARC Monographs, 10:153-161 (1976)

⁴⁸ Grove, Quart. Rev., 17:1 (1963)

⁴⁹ Huber, Antibiotics, Vol. 3, J. W. Corcoran, F. E. Hahn, Eds. (Springer-Verlag, New York, 1975) pp. 606-613.

⁵⁰ E. R. Townley, Anal. Profiles Drug Subs., 8:219-249 (1979).

⁵¹ Green, Protective Groups in Organic Synthesis, 2^{nd} Edition, John Wiley & Sons, New York, N.Y. (1991)

All of the above publications are herein incorporated by reference in their entirety to the same extent as if each individual publication was specifically and individually indicated to be incorporated by reference in its entirety.

2. State of the Art

Macromolecular structures are a separate class of cellular, extracellular and microbial components distinct from enzymes, ion channels, receptors, etc. These macromolecules are large molecular weight molecules comprising at least two, but often more, repeating, structural subunits. Such structures are often components of cellular construction such as cell envelopes (e.g., phospholipid, sterol or lipid, protein, glycolipid, and glycoprotein components), internal cytosketal structure (e.g., fibrils, filaments, etc.), polysome membranes (e.g., cellular and extracellular), vesicles, organelles, and matrices (intracellular and extracellular). Macromolecular structures also may include structural components of microbial particles such as viruses. In other cases, these molecules may be important in maintaining or modulating cellular functions. Such structures may include proteins which contain homo- and hetero-oligomeric repeating subunits such as endogenous or exogenous transcription factors.

Many well known medicinal agents/ligands (e.g., drugs) treat pathologic conditions by targeting macromolecular structures of cells mediating the disease condition. The structural targets which bind these ligands include targets on or in endogenous mammalian cells (e.g., cancer cells) or targets on exogenous microbes containing macromolecular structures (e.g., viruses, fungi, bacteria, parasites, etc.). When bound to the targeted structure, the ligand (drug) typically modulates or disrupts the biological process/function of cells or microbes which, in some cases, can result in targeted cell or microbe death. Such modulation or disruption mediates disease conditions associated with these cells or microbes. For example, the following known medicinal agents target different macromolecular structures in providing therapeutic treatment for the disease conditions indicated:

| | Macromolecular | |
|---|---|---|
| | Structure Targeted | Disease Condition |
| Drug | by Drug | Treated |
| Amphotericin B | cell wall sterol | fungal infections |
| | component (principally | (candidiasis, asperigil |
| | ergosterol) of fungi | liusis, crytococcosis, |
| | | etc.) |
| Polymyxin and | bacterial cell wall | Gram negative bacterial |
| colistin | | infections |
| | | (E. coli, Pseudomonas, |
| | | etc.) |
| Pirodivir and | capsid protein of | picornaviral infections |
| Pleconaril | picornaviruses | (rhinovirus, hepatitis A, |
| | | echovirus meningitis, |
| | | etc.) |
| Docetaxel, Paclitaxol | tubulin and/or | Cancer |
| (taxol) | microtubules | (breast and ovarian |
| | | carcinomas) |
| Griseofulvin | tubulin and/or | fungal infections |
| | microtubules | |
| Colchicine | tublin and/or | gout, gouty arthritis |
| | microtubules | |
| Vinblastine, | tubulin and/or | Cancer |
| Vincristine, | microtubules | (Hodgkin's disease, |
| Vinorelbine, and | | other lymophomas, |
| Vindesine | | testicular carcinomas, |
| | | etc.) |

Notwithstanding the above, existing drugs which target macromolecular structures often have many disadvantages such as low potency, toxicity, resistance, etc. For example, taxol and its derivatives are potent anti-neoplastic agents. While these compounds are effective in stabilizing microtubules and disrupting cellular mitosis, in clinical treatment of carcinomas, P-glycoprotein-mediated drug resistance can limit the efficacy of such agents. Such is also the case for vinblastine and other vinca alkaloid drugs.

In another case, the perceived mode of action for the anti-fungal agent Amphotericin B is through complexation with the sterol ergosterol within the fungal cell membrane. This event increases the permeability of the fungal cell membrane, leading to loss of essential intracellular components. However, notwithstanding its anti-fungal activity, Amphotericin B causes nephrotoxicity and neurotoxicity in the treatment of fungal infections.

Further, pirodavir is an antiviral agent which binds to the capsid protein of picornaviruses and is proposed to disrupt the host cell-entry process of virons. However, low potency can limit the clinical efficacy of this anti-viral agent.

Accordingly, drugs which mediate pathologic conditions by targeting macromolecular structures which drugs are more potent, less susceptible to resistance mechanisms, and/or less toxic would be particularly beneficial.

### SUMMARY OF THE INVENTION

This invention is directed to novel multibinding compounds which bind macromolecular structures including cellular, extracellular, and microbial components derived from mammalian cells and microbes such as viruses, bacteria, fungi, parasites, yeasts, and the like (“cells” as defined below). The binding of these compounds to such macromolecular structures can be used to treat pathologic conditions mediated by such cells.

Accordingly, in one of its composition aspects, this invention is directed to a multibinding compound and salts thereof comprising 2 to 10 ligands which may be the same or different and which are covalently attached to a linker or linkers which may be the same or different, each of said ligands comprising a ligand domain capable of binding to a macromolecular structure of a cell with the proviso excluding multimeric compounds having DNA binding as a primary mode of action.

Multimeric compounds having a primary mode of action based on binding to DNA are preferably characterized by dissociation constants for DNA/multimeric compounds binding of less than 1 mM.

The multibinding compounds of this invention are preferably represented by formula I:

(L)ₚ(X)_{q} I

wherein each L is independently selected from ligands comprising a ligand domain capable of binding to a macromolecular structure of a cell; each X is independently a linker; p is an integer of from 2 to 10; q is an integer of from 1 to 20 and salts thereof with the proviso excluding multimeric compounds having DNA binding as a primary mode of action.

Preferably, q is <p.

Preferably, binding of the multibinding compounds to the cell relates to cells which mediate mammalian or avian pathologic conditions and such binding modulates these conditions.

In another of its composition aspects, this invention is directed to a pharmaceutical composition comprising a pharmaceutically acceptable excipient and an effective amount of a multibinding compound or a pharmaceutically acceptable salt thereof comprising 2 to 10 ligands which may be the same or different and which are covalently attached to a linker or linkers which may be the same or different, each of said ligands comprising a ligand domain capable of binding to a macromolecular structure of a cell mediating mammalian or avian pathologic conditions thereby inhibiting the pathologic condition with the proviso excluding multimeric compounds having DNA binding as a primary mode of action.

In still another of its composition aspects, this invention is directed to a pharmaceutical composition comprising a pharmaceutically acceptable excipient and an effective amount of a multibinding compound represented by formula I:

(L)ₚ(X)_{q} I

wherein each L is independently selected from ligands comprising a ligand domain capable of binding to one or more macromolecular structures of a cell mediating mammalian or avian pathologic conditions; each X is independently a linker; p is an integer of from 2 to 10; q is an integer of from 1 to 20 and pharmaceutically acceptable salts thereof with the proviso excluding multimeric compounds having DNA binding as a primary mode of action.

Preferably, q is <p.

In one of its method aspects, this invention is directed to a method for treating a mammalian or avian pathologic condition mediated by cells comprising macromolecular structures which method comprises administering to said mammal or avian an effective amount of a pharmaceutical composition comprising a pharmaceutically acceptable excipient and a multibinding compound or a pharmaceutically acceptable salt thereof comprising 2 to 10 ligands which may be the same or different and which are covalently attached to a linker or linkers which may be the same or different, each of said ligands comprising a ligand domain capable of binding to a macromolecular structure of the cell(s) mediating mammalian or avian pathologic conditions with the proviso excluding multimeric compounds having DNA binding as a primary mode of action.

In another of its method aspects, this invention is directed to a method for treating a mammalian or avian pathologic condition mediated by cells comprising macromolecular structures which method comprises administering to said mammal or avian an effective amount of a pharmaceutical composition comprising a pharmaceutically acceptable excipient and a multibinding compound represented by formula I:

(L)ₚ(X)_{q} I

wherein each L is independently selected from ligands comprising a ligand domain capable of binding to one or more macromolecular structures of a cell mediating mammalian pathologic conditions; each X is independently a linker; p is an integer of from 2 to 10; q is an integer of from 1 to 20 and pharmaceutically acceptable salts thereof with the proviso excluding multimeric compounds having DNA binding as a primary mode of action.

Preferably, q is <p.

This invention is also directed to general synthetic methods for generating large libraries or collections of diverse multimeric compounds which bind macromolecular structures which compounds are candidates for possessing multibinding properties. In one embodiment, the general synthetic methods employ combinatorial aspects to provide for libraries of multimeric compounds which bind macromolecular structures which compounds can then be assayed for multibinding properties. In another embodiment, a collection of multimeric compounds is prepared and processed through an iterative process to determine those molecular constraints necessary to impart multibinding properties.

In the library aspect, diverse multimeric compound libraries provided by this invention are synthesized by combining a linker or linkers (i.e., a library of linkers) with a macromolecular ligand or ligands (i.e., a library of macromolecular ligands) targeting macromolecular structures of a cell to provide for a library of multimeric compounds wherein the linker and ligand each have complementary functional groups permitting covalent linkage. The library of linkers is preferably selected to have diverse properties such as valency, linker length, linker geometry and rigidity, hydrophilicity or hydrophobicity, amphiphilicity, acidity, basicity, polarization and polarizability. The library of macromolecular ligands is preferably selected to have diverse attachment points on the same ligand, different functional groups at the same site of otherwise the same ligand, different ligands and the like.

This invention is also directed to libraries of diverse multimeric compounds which compounds are candidates for possessing multibinding properties against macromolecular receptors. These libraries are prepared via the methods described above and permit the rapid and efficient evaluation (screening) of what molecular constraints impart multibinding properties to a macromolecular ligand or a class of ligands targeting a macromolecular receptor.

This invention is still further directed to iterative methods to determine those molecular constraints necessary to impart multibinding properties.

Accordingly, in one of its method aspects, this invention is directed to a method for identifying multimeric compounds possessing multibinding properties against macromolecular structures which method comprises:

(a) identifying a macromolecular ligand or a mixture of macromolecular ligands wherein each ligand contains at least one reactive functionality;

(b) identifying a library of linkers wherein each linker in said library comprises at least two functional groups having complementary reactivity to at least one of the reactive functional groups of the macromolecular ligand;

(c) preparing a multimeric compound library by combining at least two stoichiometric equivalents of the macromolecular ligand or mixture of ligands identified in (a) with the library of linkers identified in (b) under conditions wherein the complementary functional groups react to form a covalent linkage between said linker and at least two of said ligands; and

(d) assaying the multimeric compounds produced in (c) above to identify compounds possessing multibinding properties against macromolecular receptors.

In another of its method aspects, this invention is directed to a method for identifying multimeric compounds possessing multibinding properties against macromolecular structures which method comprises:

(a) identifying a library of macromolecular ligands wherein each ligand contains at least one reactive functionality;

(b) identifying a linker or mixture of linkers wherein each linker comprises at least two functional groups having complementary reactivity to at least one of the reactive functional groups of the macromolecular ligand;

(c) preparing a multimeric compound library by combining at least two stoichiometric equivalents of the library of macromolecular ligands identified in (a) with the linker or mixture of linkers identified in (b) under conditions wherein the complementary functional groups react to form a covalent linkage between said linker and at least two of said macromolecular ligands; and

(d) assaying the multimeric compounds produced in (c) above to identify multimeric compounds possessing multibinding properties against macromolecular receptors.

The preparation of the multimeric compound library is achieved by either the sequential or concurrent combination of the two or more stoichiometric equivalents of the ligands identified in (a) with the linkers identified in (b). Sequential addition of macromolecular ligands is preferred when a mixture of different macromolecular ligands is employed to ensure that heterodimeric or multimeric compounds are prepared. Concurrent addition of the macromolecular ligands is preferred when it is desired that at least a portion of the to-be-prepared multimeric compounds will be homomultimeric compounds.

The assay protocols recited in (d) can be conducted on the multimeric compound library produced in (c) above, or preferably, each member of the library can first be isolated, for example, by preparative liquid chromatography mass spectrometry (LCMS) and then assayed.

In one of its composition aspects, this invention is directed to a library of multimeric compounds which may possess multivalent properties against macromolecular receptors which library is prepared by the method comprising:

(a) identifying a macromolecular ligand or a mixture of ligands wherein each macromolecular ligand contains at least one reactive functionality;

(b) identifying a library of linkers wherein each linker in said library comprises at least two functional groups having complementary reactivity to at least one of the reactive functional groups of the macromolecular ligand; and

(c) preparing a multimeric compound library by combining at least two stoichiometric equivalents of the macromolecular ligand or mixture of ligands identified in (a) with the library of linkers identified in (b) under conditions wherein the complementary functional groups react to form a covalent linkage between said linker and at least two of said macromolecular ligands.

In another of its composition aspects, this invention is directed to a library of multimeric compounds which may possess multivalent properties against macromolecular receptors which library is prepared by the method comprising:

(a) identifying a library of macromolecular ligands wherein each ligand contains at least one reactive functionality;

(b) identifying a linker or mixture of linkers wherein each linker comprises at least two functional groups having complementary reactivity to at least one of the reactive functional groups of the macromolecular ligand; and

(c) preparing a multimeric compound library by combining at least two stoichiometric equivalents of the library of macromolecular ligands identified in (a) with the linker or mixture of linkers identified in (b) under conditions wherein the complementary functional groups react to form a covalent linkage between said linker and at least two of said macromolecular ligands.

In a preferred embodiment, the library of linkers employed in either the methods or the library aspects of this invention is selected from the group comprising flexible linkers, rigid linkers, hydrophobic linkers, hydrophilic linkers, linkers of different geometry, acidic linkers, basic linkers, linkers of different polarization and/or polarizability, and amphiphilic linkers. For example, in one embodiment, each of the linkers in the linker library may comprise linkers of different chain length and/or having different complementary reactive groups. Such linker lengths can preferably range from about 2 to 100 Å.

In another preferred embodiment, the macromolecular ligand or mixture of ligands is selected to have reactive functionality at different sites on said ligands in order to provide for a range of orientations of said ligand on said multimeric compounds. Such reactive functionality includes, by way of example, carboxylic acids, carboxylic acid halides, carboxyl esters, amines, halides, pseudohalides, isocyanates, vinyl unsaturation, ketones, aldehydes, thiols, alcohols, boronates, anhydrides, and precursors thereof. It is understood, of course, that the reactive functionality on the macromolecular ligand is selected to be complementary to at least one of the reactive groups on the linker so that a covalent linkage can be formed between the linker and the macromolecular ligand.

In other embodiments, the multimeric compound is homomeric (i.e., each of the macromolecular ligands is the same, although it may be attached at different points) or heteromeric (i.e., at least one of the macromolecular ligands is different from the other macromolecular ligands).

In addition to the combinatorial methods described herein, this invention provides for an interative process for rationally evaluating what molecular constraints impart multibinding properties to a class of multimeric compounds or ligands targeting a macromolecular receptor. Specifically, this method aspect is directed to a method for identifying multimeric compounds possessing multibinding properties against macromolecular receptors which method comprises:

(a) preparing a first collection or iteration of multimeric compounds which is prepared by contacting at least two stoichiometric equivalents of a macromolecular ligand or mixture of ligands which target a macromolecular receptor with a linker or mixture of linkers wherein said ligand or mixture of ligands comprises at least one reactive functionality and said linker or mixture of linkers comprises at least two functional groups having complementary reactivity to at least one of the reactive functional groups of the macromolecular ligand wherein said contacting is conducted under conditions wherein the complementary functional groups react to form a covalent linkage between said linker and at least two of said ligands;

(b) assaying said first collection or iteration of multimeric compounds to assess which if any of said multimeric compounds possess multibinding properties against macromolecular structures;

(c) repeating the process of (a) and (b) above until at least one multimeric compound is found to possess multibinding properties against macromolecular structures;

(d) evaluating what molecular constraints imparted multibinding properties to the multimeric compound or compounds found in the first iteration recited in (a)-(c) above;

(e) creating a second collection or iteration of multimeric compounds which elaborates upon the particular molecular constraints imparting multibinding properties to the multimeric compound or compounds found in said first iteration;

(f) evaluating what molecular constraints imparted enhanced multibinding properties to the multimeric compound or compounds found in the second collection or iteration recited in (e) above;

(g) optionally repeating steps (e) and (f) to further elaborate upon said molecular constraints.

Preferably, steps (e) and (f) are repeated at least two times, more preferably at from 2-50 times, even more preferably from 3 to 50 times, and still more preferably at least 5-50 times.

### BRIEF DESCRIPTION OF THE DRAWINGS

FIGS. 1-11 and 43-44 illustrate numerous reaction schemes suitable for preparing linkers and, hence, multibinding compounds of this invention.

FIG. 12 illustrates examples of multibinding compounds comprising 2 ligands attached in different formats to a linker.

FIG. 13 illustrates examples of multibinding compounds comprising 3 ligands attached in different formats to a linker.

FIG. 14 illustrates examples of multibinding compounds comprising 4 ligands attached in different formats to a linker.

FIG. 15 illustrates examples of multibinding compounds comprising greater than 4 ligands attached in different formats to a linker.

FIGS. 16 and 17 illustrate examples of bivalent and trivalent pleconaril oligomers.

FIG. 18 illustrates examples for the formation of such multibinding pleconaril compounds illustrated in FIG. 16 and FIG. 17.

FIGS. 19-25 illustrate several polyene macrolide antifungal agents and convenient methods for their synthesis into the multibinding compounds of this invention.

FIG. 26 illustrates the structures of several microtubule structures.

FIG. 27 illustrates the SAR for Taxol and points on the molecule subject to modification.

FIGS. 28-30 illustrate the structures of several tubulin polymerization inhibitors.

FIG. 31 illustrates different combrestatin dimers wherein, in this figure, the oval shape structure linking the dimers refers to a conventional linker group.

FIG. 32 illustrates different 2-phenyl-1,8-naphthyridin-4-one dimers wherein, in this figure, the oval shape structure linking the dimers refers to a conventional linker group.

FIG. 33 illustrates the structures of several actin binders.

FIGS. 34-38 illustrate the structures of several taxol-based monomers and dimers.

FIG. 39 illustrates the structures of several taxol-based trimers.

FIG. 40 illustrates different 2-phenyl-1,8-naphthyridin-4-one dimers and monomers useful therewith wherein, in this figure, the oval shape structure linking the dimers refers to a conventional linker group.

FIG. 41 illustrates a colchicine-based dimer and monomers useful therewith wherein, in this figure, the oval shape structure linking the dimers refers to a conventional linker group.

FIG. 42 illustrates noscapine monomer and positions available thereon to form dimers, trimers and higher multibinding compounds as per this invention. In this figure, the oval shape structure linking the dimers refers to a conventional linker group.

### DETAILED DESCRIPTION OF THE INVENTION

Ligand interactions with macromolecular structures of cells are controlled by molecular interaction/recognition between the macromolecular ligand and the structure. In turn, such interaction can result in modulation or disruption of the biological processes/functions of these cells and, in some cases, leads to cell death. Accordingly, when cells mediate mammalian pathologic conditions, interactions of the macromolecular ligand with the cellular or microbial macromolecular structure can be used to treat these conditions.

The interaction of a macromolecular structure and a macromolecular ligand may be described in terms of “affinity” and “specificity”. The affinity and specificity of any given ligand/macromolecular structure interaction are dependent upon the complementarity of molecular binding surfaces and the energetic costs of complexation. Affinity is sometimes quantified by the equilibrium constant of complex formation.

Specificity relates to the difference in affinity between the same ligand binding to different ligand binding sites on the same or different macromolecular structures.

The multibinding compounds of this invention are capable of acting as multibinding agents and the surprising activity of these compounds arises at least in part from their ability to bind in a multivalent manner with one or more macromolecular structures of cells. Multivalent binding interactions are characterized by the concurrent interaction of multiple ligands with multiple ligand binding sites on one or more macromolecular structures. Multivalent interactions differ from collections of individual monovalent interactions by imparting enhanced biological and/or therapeutic effect. Examples of multivalent binding interactions (e.g., trivalent) relative to monovalent binding interactions are shown below:

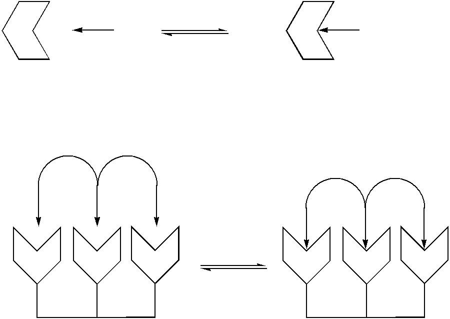

Just as multivalent binding can amplify binding affinities, it can also amplify differences in binding affinities, resulting in enhanced binding specificity as well as affinity.

Prior to discussing this invention in further detail, the following terms will first be defined.

The term “library” refers to at least 3, preferably from 10² to 10⁹ and more preferably from 10² to 10⁴ multimeric compounds. Preferably, these compounds are prepared as a multiplicity of compounds in a single solution or reaction mixture which permits facile synthesis thereof In one embodiment, the library of multimeric compounds can be directly assayed for multibinding properties. In another embodiment, each member of the library of multimeric compounds is first isolated and, optionally, characterized. This member is then assayed for multibinding properties.

The term “collection” refers to a set of multimeric compounds which are prepared either sequentially or concurrently (e.g., combinatorially). The collection comprises at least 2 members; preferably from 2 to 10⁹ members and still more preferably from 10 to 10⁴ members.

The term “multimeric compound” refers to compounds comprising from 2 to 10 macromolecular ligands covalently connected through at least one linker which compounds may or may not possess multibinding properties (as defined herein).

The term “alkyl” refers to a monoradical branched or unbranched saturated hydrocarbon chain preferably having from 1 to 40 carbon atoms, more preferably 1 to 10 carbon atoms, and even more preferably 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group as defined above, having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkylene” refers to a diradical of a branched or unbranched saturated hydrocarbon chain, preferably having from 1 to 40 carbon atoms, more preferably 1 to 10 carbon atoms and even more preferably 1 to 6 carbon atoms. This term is exemplified by groups such as methylene (—CH₂—, ethylene (—CH₂CH₂—), the propylene isomers (e.g., —CH₂CH₂CH₂— and —CH(CH₃)CH₂—) and the like.

The term “substituted alkylene” refers to an alkylene group, as defined above, having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Additionally, such substituted alkylene groups include those where 2 substituents on the alkylene group are fused to form one or more cycloalkyl, substituted cycloalkyl, cycloalkenyl, substituted cycloalkenyl, aryl, heterocyclic or heteroaryl groups fused to the alkylene group. Preferably such fused groups contain from 1 to 3 fused ring structures.

The term “alkaryl” refers to the groups -alkylene-aryl and -substituted alkylene-aryl where alkylene, substituted alkylene and aryl are defined herein. Such alkaryl groups are exemplified by benzyl, phenethyl and the like.

The term “alkoxy” refers to the groups alkyl-O—, alkenyl-O—, cycloalkyl-O—, cycloalkenyl-O—, and alkynyl-O—, where alkyl, alkenyl, cycloalkyl, cycloalkenyl, and alkynyl are as defined herein. Preferred alkoxy groups are alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkylalkoxy” refers to the groups -alkylene-O-alkyl, alkylene-O-substituted alkyl, substituted alkylene-O-alkyl and substituted alkylene-O-substituted alkyl wherein alkyl, substituted alkyl, alkylene and substituted alkylene are as defined herein. Preferred alkylalkoxy groups are alkylene-O-alkyl and include, by way of example, methylenemethoxy (—CH₂OCH₃), ethylenemethoxy (—CH₂CH₂OCH₃), n-propylene-iso-propoxy (—CH₂CH₂CH₂OCH(CH₃)₂), methylene-t-butoxy (—CH₂—O—C(CH₃)₃) and the like.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein. Preferred alkylthioalkoxy groups are alkylene-S-alkyl and include, by way of example, methylenethiomethoxy (—CH₂SCH₃), ethylenethiomethoxy (—CH₂CH₂SCH₃), n-propylene-iso-thiopropoxy (—CH₂CH₂CH₂SCH(CH₃)₂), methylene-t-thiobutoxy (—CH₂SC(CH₃)₃) and the like.

The term “alkenyl” refers to a monoradical of a branched or unbranched unsaturated hydrocarbon group preferably having from 2 to 40 carbon atoms, more preferably 2 to 10 carbon atoms and even more preferably 2 to 6 carbon atoms and having at least 1 and preferably from 1-6 sites of vinyl unsaturation. Preferred alkenyl groups include ethenyl (—CH═CH₂), n-propenyl (—CH₂CH═CH₂), iso-propenyl (—C(CH₃)═CH₂), and the like.

The term “substituted alkenyl” refers to an alkenyl group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkenylene” refers to a diradical of a branched or unbranched unsaturated hydrocarbon group preferably having from 2 to 40 carbon atoms, more preferably 2 to 10 carbon atoms and even more preferably 2 to 6 carbon atoms and having at least 1 and preferably from 1-6 sites of vinyl unsaturation. This term is exemplified by groups such as ethenylene (—CH═CH—), the propenylene isomers (e.g., —CH₂CH═CH— and —C(CH₃)═CH—) and the like.

The term “substituted alkenylene” refers to an alkenylene group as defined above having from 1 to 5 substituents, and preferably from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Additionally, such substituted alkenylene groups include those where 2 substituents on the alkenylene group are fused to form one or more cycloalkyl, substituted cycloalkyl, cycloalkenyl, substituted cycloalkenyl, aryl, heterocyclic or heteroaryl groups fused to the alkenylene group.

The term “alkynyl” refers to a monoradical of an unsaturated hydrocarbon preferably having from 2 to 40 carbon atoms, more preferably 2 to 20 carbon atoms and even more preferably 2 to 6 carbon atoms and having at least 1 and preferably from 1-6 sites of acetylene (triple bond) unsaturation. Preferred alkynyl groups include ethynyl (—C≡CH), propargyl (—CH₂C≡CH) and the like.

The term “substituted alkynyl” refers to an alkynyl group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkynylene” refers to a diradical of an unsaturated hydrocarbon preferably having from 2 to 40 carbon atoms, more preferably 2 to 10 carbon atoms and even more preferably 2 to 6 carbon atoms and having at least 1 and preferably from 1-6 sites of acetylene (triple bond) unsaturation. Preferred alkynylene groups include ethynylene (—C≡C—), propargylene (—CH₂C≡C—) and the like.

The term “substituted alkynylene” refers to an alkynylene group as defined above having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “acyl” refers to the groups HC(O)—, alkyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)— where alkyl, substituted alkyl, cycloalkyl, substituted cycloalkyl, cycloalkenyl, substituted cycloalkenyl, aryl, heteroaryl and heterocyclic are as defined herein.

The term “acylamino” or “aminocarbonyl” refers to the group —C(O)NRR where each R is independently hydrogen, alkyl, substituted alkyl, aryl, heteroaryl, heterocyclic or where both R groups are joined to form a heterocyclic group (e.g., morpholino) wherein alkyl, substituted alkyl, aryl, heteroaryl and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” or “alkoxycarbonylamino” refers to the group —NRC(O)OR where each R is independently hydrogen, alkyl, substituted alkyl, aryl, heteroaryl, or heterocyclic wherein alkyl, substituted alkyl, aryl, heteroaryl and heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Preferred aryls include phenyl, naphthyl and the like.

Unless otherwise constrained by the definition for the aryl substituent, such aryl groups can optionally be substituted with from 1 to 5 substituents, preferably 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy.

The term “aryloxy” refers to the group aryl-O— wherein the aryl group is as defined above including optionally substituted aryl groups as also defined above.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂.

The term “substituted amino refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic provided that both R's are not hydrogen.

The term “carboxyalkyl” or “alkoxycarbonyl” refers to the groups “—C(O)O-alkyl”, “—C(O)O-substituted alkyl”, “—C(O)O-cycloalkyl”, “—C(O)O-substituted cycloalkyl”, “—C(O)O-alkenyl”, “—C(O)O-substituted alkenyl”, “—C(O)O-alkynyl” and “—C(O)O-substituted alkynyl” where alkyl, substituted alkyl, cycloalkyl, substituted cycloalkyl, alkenyl, substituted alkenyl, alkynyl and substituted alkynyl alkynyl are as defined herein.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “cycloalkylene” refers to the diradical derived from cycloalkyl as defined above and is exemplified by 1,1-cyclopropylene, 1,2-cyclobutylene, 1,4-cyclohexylene and the like.

The term “substituted cycloalkyl” refers to cycloalkyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkylene” refers to the diradical derived from substituted cycloalkyl as defined above.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl and the like.

The term “cycloalkenylene” refers to the diradical derived from cycloalkenyl as defined above and is exemplified by 1,2-cyclobut-1-enylene, 1,4-cyclohex-2-enylene and the like.

The term “substituted cycloalkenyl” refers to cycloalkenyl groups having from 1 to 5 substituents, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkenylene” refers to the diradical derived from substituted cycloalkenyl as defined above.

The term “halo” or “halogen” refers to fluoro, chloro, bromo and iodo.

The term “pseudohalide” refers to functional groups which react in displacement reactions in a manner similar to a halogen. Such functional groups include, by way of example, mesyl, tosyl, azido and cyano groups.

The term “heteroaryl” refers to an aromatic group of from 1 to 15 carbon atoms and 1 to 4 heteroatoms selected from oxygen, nitrogen and sulfur within at least one ring (if there is more than one ring).

Unless otherwise constrained by the definition for the heteroaryl substituent, such heteroaryl groups can be optionally substituted with 1 to 5 substituents, preferably 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Preferred aryl substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). Preferred heteroaryls include pyridyl, pyrrolyl and furyl.

The term “heteroaryloxy” refers to the group heteroaryl-O—.

The term “heteroarylene” refers to the diradical group derived from heteroaryl (including substituted heteroaryl), as defined above, and is exemplified by the groups 2,6-pyridylene, 2,4-pyridiylene, 1,2-quinolinylene, 1,8-quinolinylene, 1,4-benzofuranylene, 2,5-pyridnylene, 2,5-indolenyl and the like.

The term “heterocycle” or “heterocyclic” refers to a monoradical saturated unsaturated group having a single ring or multiple condensed rings, from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Such heterocyclic groups can have a single ring or multiple condensed rings. Preferred heterocyclics include morpholino, piperidinyl, and the like.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

A preferred class of heterocyclics include “crown compounds” which refers to a specific class of heterocyclic compounds having one or more repeating units of the formula [—(CH₂)ₘY—] where m is ≧2, and Y at each separate occurrence can be O, N, S or P. Examples of crown compounds include, by way of example only, [—(CH₂)₃—NH—]₃, [—((CH₂)₂—O)₄—((CH₂)₂—NH—)₂] and the like. Typically such crown compounds can have from 4 to 10 heteroatoms and 8 to 40 carbon atoms.

The term “heterocyclooxy” refers to the group heterocyclic-O—.

The term “thioheterocyclooxy” refers to the group heterocyclic-S—.

The term “heterocyclene” refers to the diradical group formed from a heterocycle, as defined herein, and is exemplified by the groups 2,6-morpholino, 2,5-morpholino and the like.

The term “oxyacylamino” or “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “spiro-attached cycloalkyl group” refers to a cycloalkyl group attached to another ring via one carbon atom common to both rings.

The term “thiol” refers to the group —SH.

The term “thioalkoxy” refers to the group —S-alkyl.

The term “substituted thioalkoxy” refers to the group —S-substituted alkyl.

The term “thioaryloxy” refers to the group aryl-S— wherein the aryl group is as defined above including optionally substituted aryl groups also defined above.

The term “thioheteroaryloxy” refers to the group heteroaryl-S— wherein the heteroaryl group is as defined above including optionally substituted aryl groups as also defined above.

As to any of the above groups which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

The term “pharmaceutically acceptable salt” refers to salts which retain the biological effectiveness and properties of the multibinding compounds of this invention and which are not biologically or otherwise undesirable. In many cases, the multibinding compounds of this invention are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “protecting group” or “blocking group” refers to any group which when bound to one or more hydroxyl, thiol, amino or carboxyl groups of the compounds (including intermediates thereof) prevents reactions from occurring at these groups and which protecting group can be removed by conventional chemical or enzymatic steps to reestablish the hydroxyl, thiol, amino or carboxyl group. The particular removable blocking group employed is not critical and preferred removable hydroxyl blocking groups include conventional substituents such as allyl, benzyl, acetyl, chloroacetyl, thiobenzyl, benzylidine, phenacyl, t-butyl-diphenylsilyl and any other group that can be introduced chemically onto a hydroxyl functionality and later selectively removed either by chemical or enzymatic methods in mild conditions compatible with the nature of the product.

Preferred removable thiol blocking groups include disulfide groups, acyl groups, benzyl groups, and the like.

Preferred removable amino blocking groups include conventional substituents such as t-butyoxycarbonyl (t-BOC), benzyloxycarbonyl (CBZ), fluorenylmethoxycarbonyl (FMOC), allyloxycarbonyl (ALOC), and the like which can be removed by conventional conditions compatible with the nature of the product.

Preferred carboxyl protecting groups include esters such as methyl, ethyl, propyl, t-butyl etc. which can be removed by mild conditions compatible with the nature of the product.

The term “optional” or “optionally” means that the subsequently described event, circumstance or substituent may or may not occur, and that the description includes instances where said event or circumstance occurs and instances where it does not.

The term “ligand” or “macromolecular ligand” as used herein denotes a compound that is a binding partner for a macromolecular structure and is bound thereto by complementarity. The specific region or regions of the macromolecular ligand that is (are) recognized by the macromolecular structure is designated as the “ligand domain” or the “macromolecular ligand domain”. A macromolecular ligand may be either capable of binding to a macromolecular structure by itself, or may require the presence of one or more non-ligand components for binding (e.g., Ca⁺², Mg⁺² or a water molecule is required for the binding of a macromolecular ligand to various ligand binding sites). It should be appreciated that the term “macromolecular ligand” does not infer that the ligand is macromolecular in size; rather, the term refers to the fact that the ligand targets a macromolecular receptor. As a point of fact, the ligand may or may not be macromolecular in size.

Examples of macromolecular ligands useful in this invention are given below. Those skilled in the art will appreciate that portions of the macromolecular ligand structure that are not essential for specific molecular recognition and binding activity may be varied substantially, replaced or substituted with unrelated structures (for example, with ancillary groups as defined below) and, in some cases, omitted entirely without affecting the binding interaction. The primary requirement for a macromolecular ligand is that it has a ligand domain as defined above. It is understood that the term macromolecular ligand is not intended to be limited to compounds known to be useful in binding macromolecular structures (e.g., known drugs). Those skilled in the art will understand that the term macromolecular ligand can equally apply to a molecule that is not normally associated with macromolecular structure binding properties. The primary requirement for a macromolecular ligand as defined herein is that it has a ligand domain as defined above. In addition, it should be noted that macromolecular ligands that exhibit marginal activity or lack useful activity as monomers can be highly active as multivalent compounds because of the benefits conferred by multivalency.

The term “multibinding compound cr agent” refers to a compound that is capable of multivalency, as defined below, and which has 2-10 macromolecular ligands covalently bound to one or more linkers which may be the same or different. Multibinding compounds provide a biological and/or therapeutic effect greater than the aggregate of unlinked macromolecular ligands equivalent thereto which are made available for binding. That is to say that the biological and/or therapeutic effect of the macromolecular ligands attached to the multibinding compound is greater than that achieved by the same amount of unlinked macromolecular ligands made available for binding to the ligand binding sites (receptors). The phrase “increased biological or therapeutic effect” includes, for example: increased affinity, increased selectivity for target, increased specificity for target, increased potency, increased efficacy, decreased toxicity, improved duration of activity or action, increased ability to kill cells such as fungal pathogens, cancer cells, etc., decreased side effects, increased therapeutic index, improved bioavailibity, improved pharmacokinetics, improved activity spectrum, and the like. The multibinding compounds of this invention will exhibit at least one and preferably more than one of the above-mentioned affects.

The term “potency” refers to the minimum concentration at which a macromolecular ligand is able to achieve a desirable biological or therapeutic effect. The potency of a macromolecular ligand is typically proportional to its affinity for its ligand binding site. In some cases, the potency may be non-linearly correlated with its affinity. In comparing the potency of two drugs, e.g., a multibinding agent and the aggregate of its unlinked macromolecular ligand, the dose-response curve of each is determined under identical test conditions (e.g., in an in vitro or in vivo assay, in an appropriate animal model). The finding that the multbinding agent produces an equivalent biological or therapeutic effect at a lower concentration than the aggregate unlinked macromolecular ligand is indicative of enhanced potency.

The term “univalency” as used herein refers to a single binding interaction between one macromolecular ligand as defined herein with one macromolecular ligand binding site as defined herein. It should be noted that a compound having multiple copies of a macromolecular ligand (or ligands) exhibit univalency when only one macromolecular ligand is interacting with a macromolecular ligand binding site. Examples of univalent interactions are depicted below.

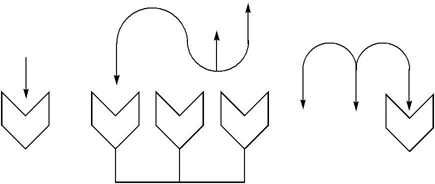

The term “multivalency” as used herein refers to the concurrent binding of from 2 to 10 linked macromolecular ligands (which may be the same or different) and two or more corresponding receptors (macromolecular ligand binding sites) on the macromolecular structures which structures may be the same or different.

For example, two macromolecular ligands connected through a linker that bind concurrently to two macromolecular ligand binding sites would be considered as bivalency; three macromolecular ligands thus connected would be an example of trivalency. An example of trivalent binding, illustrating a multibinding compound bearing three macromolecular ligands versus a monovalent binding interaction, is shown below:

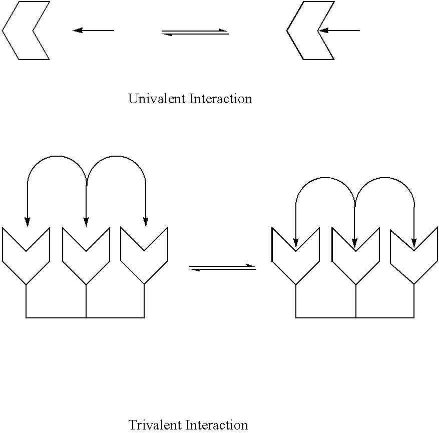

It should be understood that not all compounds that contain multiple copies of a macromolecular ligand attached to a linker or to linkers necessarily exhibit the phenomena of multivalency, i.e., that the biological and/or therapeutic effect of the multibinding agent is greater than the sum of the aggregate of unlinked macromolecular ligands made available for binding to the macromolecular ligand binding site (receptor). For multivalency to occur, the macromolecular ligands that are connected by a linker or linkers have to be presented to their ligand binding sites by the linker(s) in a specific manner in order to bring about the desired ligand-orienting result, and thus produce a multibinding event.

The term “selectivity” or “specificity” is a measure of the binding preferences of a macromolecular ligand for different macromolecular ligand binding sites (receptors). The selectivity of a macromolecular ligand with respect to its target macromolecular ligand binding site relative to another macromolecular ligand binding site is given by the ratio of the respective values of K_{d} (i.e., the dissociation constants for each ligand-receptor complex) or, in cases where a biological effect is observed below the K_{d}, the ratio of the respective EC₅₀S (i.e., the concentrations that produce 50% of the maximum response for the macromolecular ligand interacting with the two distinct ligand binding sites (receptors)).

The term “ligand binding site” or “macromolecular ligand binding site” denotes the site on a macromolecular structure that recognizes a macromolecular ligand domain and provides a binding partner for the macromolecular ligand. The macromolecular ligand binding site may be defined by monomeric or multimeric structures. This interaction may be capable of producing a unique biological effect, for example, agonism, antagonism, modulatory effects, may maintain an ongoing biological event, and the like. For the purposes of this invention, the macromolecular ligand/ligand domain, and the macromolecular ligand binding site are not DNA, RNA, an antibody, an antibody domain, or a fragment of an antibody.

It should be recognized that the ligand binding sites of the macromolecular structures that participate in biological multivalent binding interactions are constrained to varying degrees by their intra- and inter-molecular associations (e.g., such macromolecular structures may be covalently joined to a single structure, noncovalently associated in a multimeric structure, embedded in a membrane or polymeric matrix, and so on) and therefore have less translational and rotational freedom than if the same structures were present as monomers in solution.

The terms “agonism” and “antagonism” are well known in the art. The term “modulatory effect” refers to the ability of the ligand to change the activity of an agonist or antagonist through binding to a ligand binding site.

The term “macromolecular structures” refer to cellular, extracellular and/or microbial components comprising 2 or more repeating structural subunits. Such molecules are often components of cellular construction such as components of cell envelops (e.g., phospholipid, sterol or lipid, protein, glycolipid, and glycoprotein components), internal cytosketal structures (e.g., fibrils, filaments, etc.), polysome membranes (e.g., cellular and extracellular), vesicles, organelles, and matrices (intracellular and extracellular). Macromolecular structures also may include structural components of microbial particles such as viruses. In other cases, these molecules may be important in maintaining or modulating cellular function. Such structures may include proteins which contain homo- and hetero-oligomeric repeating units such as endogenous or exogenous transcription factors. On the other hand, as used herein, macromolecular structures do not include DNA, RNA, antibodies, or antibody fragments.

The macromolecular structure targeted by the macromolecular ligand can be found on/in endogenous mammalian cells or on/in exogenous sources (e.g., bacterial, viral, fungal, parasitic, sources). Binding between the macromolecular ligands of the multibinding compounds of this invention and the ligand binding sites on the macromolecular structure result in modulation or disruption of the biological process/function of cells and, in some cases, can lead to cell death.

Examples of macromolecular structures and ligands which bind thereto include those set forth in the table below:

Multivalent Modulators Of Macromolecular Interactions

| | Current and | |
|---|---|---|
| | Potential | |
| | Therapeutic | Drugs and Other |
| Target | Indication(s) | Ligands |
| Fungal cell | Fungal infections, viral | Amphotericin, nystatin, |
| membrane | infections, Creutzfeldt- | candidacine, hamycin, |
| sterol | Jakob disease | auefungin, ascosin, |
| | | ayfattin, azacolutin, DJ- |
| | | 400B, trichomycin a, |
| | | levorin, heptamycin, |
| | | candimycin, perimycin, |
| | | vacidin A, fariefungin, |
| | | roflamycoin, roxaticin, |
| | | dermostin, aureofuscin, |
| | | retilavendomycin, |
| | | arenomycin B, |
| | | lucensomycin, pimaricin, |
| | | tetramycins A/B, tetrins |
| | | A/B, aurenin, rimocidin, |
| | | fungichromin, elizaethin, |
| | | chainin, filipins |
| | | I/II/III/IV, eurocidins |
| | | A/B, gannibamycin, |
| | | mycoticins A/B, |
| | | dermostatins A/B, |
| | | polifungin B, |
| | | aureofungin A, candidin, |
| | | mycoheptin, partricins |
| | | A/B, perimycin A, 67- |
| | | 121 A/C, lienomycin, |
| | | MS-8209, SPA-S-753, |
| | | V-28-3B |
| Fungal cell | Fungal infections | Benanomicin A, |
| wall | | pradimicins |
| mannans/ | | |
| glycomannans | | |
| Bacterial | Bacterial infections | Polymyxins, ranalexin, |
| membrane | | colistin, octapeptins, |
| | | circulin |
| Bacterial cell | Bacterial infections | Daptomycin |
| wall lipoteichoic | | |
| acid | | |
| Microbial cell | Microbial infections | Magainins, cecropins, |
| membranes | | defensins, gramicidin A, |
| | | apidaecin, bactenecin, |
| | | dermaseptin, indolicidin, |
| | | bombinins, brevinins, |
| | | esculentin, tachyplesins, |
| | | protegrins, squalamine |
| Viral capsid | Viral infections | Pirodivir, pleconaril, |
| | | WIN-54954, R-80633, |
| | | R-61837, R-77975 |
| Tubulin/ | Cancer | Vinblastine, vincristine, |
| microtubules | Hodgkin's disease | vinorelbine, vindesine, |
| Actin | Lymphoma | taxol, taxotere, |
| | Testicular carcinoma | colchicine, epothilone, |
| | Fungal infection | descodermolide, |
| | Gout | dolastatin 10, |
| | Gouty arthritis | anhydrovinblastine, |
| | Neoplasm | docetaxel, TZT-1027, |
| | | desoxyepothilone B, |
| | | vinflunine, cemadotin, |
| | | dolastatin 15, |
| | | vinorelbine, CI-980, LY- |
| | | 355703, LY-355702, |
| | | cryptophycin, LY- |
| | | 290181, RPR-112378, |
| | | sarcodictyins, T-138067, |
| | | 9-dihydrotaxanes, LL- |
| | | 15, NSC-639829, |
| | | dolaphenine, SJ-3249, |
| | | halamide, SB-T-1101, |
| | | SB-T-104221, |
| | | eleutherovin, SB-T- |
| | | 1211, 1069C85, celcein, |
| | | LS-4477, LS-4559, MPI- |
| | | 6003, halichondrin B, |
| | | DDE-313, 3-IAABU, T- |
| | | 900607, rhizoxin, |
| | | azatoxin, |
| | | palmitoylrhizoxin, |
| | | noscapine, spongistatins, |
| | | paclitaxel analogs, |
| | | aplyronine-A, GS-164, |
| | | BMS-247550, BP-179, |
| | | isohomohalichondrin B, |
| | | T-3782, oncocidin A1, |
| | | SPA-1, curacin A, NSC- |
| | | 692745, NSC-698666, |
| | | chondramide D, LU- |
| | | 110946, PNU-166945, |
| | | BMS-185660, ZYN-176, |
| | | ZYN-162, D-24851, |
| | | griseofulvin |
| Bone | Osteoporosis | Alendromate, |
| hydroxylapatite | Hypercalcemia | ibandronate, risedronate, |
| | Paget's disease | zoledronate, incadronate, |
| | | clodronate, olpadronate |
| Beta amyloid | Alzheimer's disease | Lignan glycosides, |
| | Parkinson's disease | sulfonate compounds, |
| | Neurodegenerative | LVFFA analogs, PPI- |
| | disease | 368, azo dye analogs, |
| | | PTI-00703, SIB-1848, |
| | | PPI-558, SKF-74652 |
| DNA | Cancer | Adriamycin, |
| | | daunomycin, CI-958, |
| | | valrubicin, idarubicin, |
| | | epirubicin, mitoxantrone, |
| | | zorubicin, bleomycin |
| | | A2, plicamycin, |
| | | dactinomycin, NSC- |
| | | 655649, crisnatol, |
| | | losoxantrone, |
| | | antineoplaston A10, |
| | | mitonafide, intoplicine, |
| | | annamycin, BBR-2778, |
| | | distamycin, MEN- |
| | | 10718, FCE-28102, |
| | | FCE-28164, FCE- |
| | | 25450A, PNU-151779, |
| | | PNU-166196, NSC- |
| | | 651016, tallimustine, |
| | | FCE-26644, bizelesin |
| Cell membrane | Cancer | N-1379 |
| | Microbial infections | |
| Heat shock | Cancer | Geldanamycin |
| proteins | | |
| HIV 1 p7NC | HIV infections | CI-1012, NCS-4493, |
| protein | | NSC-2065, |
| (Nucleocapsid | | azodicarbonamide |
| protein) | | |
| Grb-2 | Cancer | Bis(indolyl)- |
| | | dihydroxyquinones |

The term “inert organic solvent” or “organic solvent” means a solvent which is inert under the conditions of the reaction being described in conjunction therewith including, by way of example only, benzene, toluene, acetonitrile, tetrahydrofuran, dimethylformamide, chloroform, methylene chloride, diethyl ether, ethyl acetate, acetone, methylethyl ketone, methanol, ethanol, propanol, isopropanol, t-butanol, dioxane, pyridine, and the like. Unless specified to the contrary, the solvents used in the reactions described herein are inert solvents.

The term “treatment” refers to any treatment of a pathologic condition in a mammal or avian, particularly a human, and includes:

(i) preventing the pathologic condition from occurring in a subject which may be predisposed to the condition but has not yet been diagnosed with the condition and, accordingly, the treatment constitutes prophylactic treatment for the disease condition;

(ii) inhibiting the pathologic condition, i.e., arresting its development;

(iii) relieving the pathologic condition, i.e., causing regression of the pathologic condition; or

(iv) relieving the conditions mediated by the pathologic condition, e.g., relieving the conditions caused by an enterotoxin expressed by a microorganism but not addressing the underlining microbial infection.

The term “pathologic condition which is modulated by treatment with a ligand” covers all disease states (i.e., pathologic conditions) which are generally acknowledged in the art to be usefully treated with a ligand for a macromolecular structure in general, and those disease states which have been found to be usefully treated by a specific multibinding compound of our invention. Such disease states include, by way of example only, the treatment of a mammal afflicted with pathologic bacteria, fungal infections, cancer including breast cancer and prostrate cancer, and the like. It also covers the treatment of pathologic conditions that are not necessarily considered as pathologic conditions, for example the use of multi-binding compounds of this invention in the treatment of skin diseases, beauty aids and the like.

The term “therapeutically effective amount” refers to that amount of multibinding compound which is sufficient to effect treatment, as defined above, when administered to a mammal or avian in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “cell” refers to mammalian cells (e.g., mammalian cancer cells), virus particles, bacterial cells, fungal cells, and parasitic cells.

The term “linker”, identified where appropriate by the symbol X, refers to a group or groups that covalently links from 2 to 10 macromolecular ligands (as identified above) in a manner that provides for a compound capable of multivalency when in the presence of at least one macromolecular structure having 2 or more ligand binding sites. Among other features, the linker is a ligand-orienting entity that permits attachment of multiple copies of a macromolecular ligand (which may be the same or different) thereto. In some cases, the linker may itself be biologically active. The term “linker” does not, however, extend to cover solid inert supports such as beads, glass particles, fibers, and the like. But it is understood that the multibinding compounds of this invention can be attached to a solid support if desired. For example, such attachment to solid supports can be conducted for use in separation and purification processes and similar applications.

The extent to which multivalent binding is realized depends upon the efficiency with which the linker or linkers that joins the macromolecular ligands presents these ligands to the array of ligand binding sites on one or more macromolecular structure(s). Beyond presenting these ligands for multivalent interactions with ligand binding sites, the linker or linkers spatially constrains these interactions to occur within dimensions defined by the linker or linkers. Thus, the structural features of the linker (valency, geometry, orientation, size, flexibility, chemical composition, etc.) are features of multibinding agents that play an important role in determining their activities.

The linkers used in this invention are selected to allow multivalent binding of macromolecular ligands to any desired ligand binding sites of a macromolecular structure, whether such sites are located interiorly, both interiorly and on the periphery of the macromolecular structure, or at any intermediate position thereof. The distance between the nearest neighboring ligand domains is preferably in the range of about 2 Å to about 100 Å, more preferably in the range of about 3 Å to about 40 Å.

The macromolecular ligands are covalently attached to the linker or linkers using conventional chemical techniques providing for covalent linkage of the ligand to the linker or linkers. Reaction chemistries resulting in such linkages are well known in the art and involve the use of complementary functional groups on the linker and macromolecular ligand. Preferably, the complementary functional groups on the linker are selected relative to the functional groups available on the ligand for bonding or which can be introduced onto the ligand for bonding. Again, such complementary functional groups are well known in the art. For example, reaction between a carboxylic acid of either the linker or the ligand and a primary or secondary amine of the ligand or the linker in the presence of suitable, well-known activating agents results in formation of an amide bond covalently linking the ligand to the linker; reaction between an amine group of either the linker or the ligand and a sulfonyl halide of the ligand or the linker results in formation of a sulfonamide bond covalently linking the ligand to the linker; and reaction between an alcohol or phenol group of either the linker or the ligand and an alkyl or aryl halide of the ligand or the linker results in formation of an ether bond covalently linking the ligand to the linker.

The linker is attached to the macromolecular ligand at a position that retains ligand domain-ligand binding site interaction and specifically which permits the ligand domain of the ligand to orient itself to bind to the ligand binding site. Such positions and synthetic protocols for linkage are well known in the art. The term linker embraces everything that is not considered to be part of the ligand.

The relative orientation in which the macromolecular ligand domains are displayed derives from the particular point or points of attachment of the ligands to the linker, and on the framework geometry. The determination of where acceptable substitutions can be made on a macromolecular ligand is typically based on prior knowledge of structure-activity relationships (SAR) of the ligand and/or congeners and/or structural information about ligand-receptor complexes (e.g., X-ray crystallography, NMR, and the like). Such positions and the synthetic methods for covalent attachment are well known in the art. Following attachment to the selected linker (or attachment to a significant portion of the linker, for example 2-10 atoms of the linker), the univalent linker-ligand conjugate may be tested for retention of activity in the relevant assay. In a preferred embodiment, if the linker-ligand shows activity at a concentration of less than 10 mM, it may be considered to be acceptable for use in constructing a multi-binding compound.

Suitable linkers are discussed below.

At present, it is preferred that the multibinding agent is a bivalent compound, e.g., two ligands which are covalently linked to linker X.

Methodology

The linker, when covalently attached to multiple copies of the macromolecular ligands, provides a biocompatible, substantially non-immunogenic multibinding compound. The biological activity of the multibinding compound is highly sensitive to the valency, geometry, composition, size, flexibility or rigidity, etc. of the linker and, in turn, on the overall structure of the multibinding compound, as well as the presence or absence of anionic or cationic charge, the relative hydrophobicity/hydrophilicity of the linker, and the like on the linker. Accordingly, the linker is preferably chosen to maximize the biological activity of the multibinding compound. The linker may be chosen to enhance the biological activity of the molecule. In general, the linker may be chosen from any organic molecule construct that orients two or more macromolecular ligands to their ligand binding sites to permit multivalency. In this regard, the linker can be considered as a “framework” on which the ligands are arranged in order to bring about the desired ligand-orienting result, and thus produce a multibinding compound.

For example, different orientations can be achieved by including in the framework groups containing mono- or polycyclic groups, including aryl and/or heteroaryl groups, or structures incorporating one or more carbon-carbon multiple bonds (alkenyl, alkenylene, alkynyl or alkynylene groups). Other groups can also include oligomers and polymers which are branched- or straight-chain species. In preferred embodiments, rigidity is imparted by the presence of cyclic groups (e.g., aryl, heteroaryl, cycloalkyl, heterocyclic, etc.). In other preferred embodiments, the ring is a six or ten member ring. In still further preferred embodiments, the ring is an aromatic ring such as, for example, phenyl or naphthyl.

Different hydrophobic/hydrophilic characteristics of the linker as well as the presence or absence of charged moieties can readily be controlled by the skilled artisan. For example, the hydrophobic nature of a linker derived from hexamethylene diamine (H₂N(CH₂)₆NH₂) or related polyamines can be modified to be substantially more hydrophilic by replacing the alkylene group with a poly(oxyalkylene) group such as found in the commercially available “Jeffamines”.

Different frameworks can be designed to provide preferred orientations of the ligands. Such frameworks may be represented by using an array of dots (as shown below) wherein each dot may potentially be an atom, such as C, O, N, S, P, H, F, Cl, Br, and F or the dot may alternatively indicate the absence of an atom at that position. To facilitate the understanding of the framework structure, the framework is illustrated as a two dimensional array in the following diagram, although clearly the framework is a three dimensional array in practice:

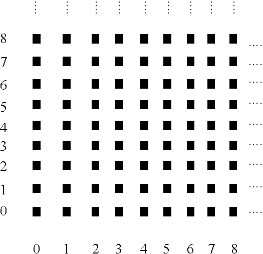

Each dot is either an atom, chosen from carbon, hydrogen, oxygen, nitrogen, sulfur, phosphorus, or halogen, or the dot represents a point in space (i.e., an absence of an atom). As is apparent to the skilled artisan, only certain atoms on the grid have the ability to act as an attachment point for the macromolecular ligands, namely, C, O, N, S and P.

Atoms can be connected to each other via bonds (single, double or triple bonds with acceptable resonance and tautomeric forms), with regard to the usual constraints of chemical bonding. Macromolecular ligands may be attached to the framework via single, double or triple bonds (with chemically acceptable tautomeric and resonance forms). Multiple ligand groups (2 to 10) can be attached to the framework such that the minimal, shortest path distance between adjacent ligand groups does not exceed 100 atoms. Preferably, the linker connections to the ligand is selected such that the maximum spatial distance between two adjacent ligands is no more than 40 Å.

An example of a linker as presented by the grid is shown below for a biphenyl construct.

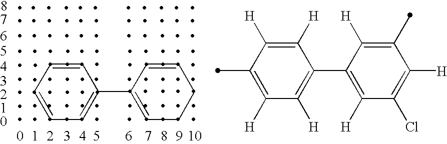

Nodes (1, 2), (2, 0), (4, 4), (5, 2), (4, 0), (6, 2), (7, 4), (9, 4), (10, 2), (9, 0), (7, 0) all represent carbon atoms. Node (10, 0) represents a chlorine atom. All other nodes (or dots) are points in space (i.e., represent an absence of atoms).

Nodes (1, 2) and (9, 4) are attachment points. Hydrogen atoms are affixed to nodes (2, 4), (4, 4), (4, 0), (2, 0), (7, 4), (10, 2) and (7, 0). Nodes (5, 2) and (6, 2) are connected by a single bond.

The carbon atoms present are connected by either a single or double bonds, taking into consideration the principle of resonance and/or tautomerism.

The intersection of the framework (linker) and the macromolecular ligand group, and indeed, the framework (linker) itself can have many different bonding patterns. Examples of acceptable patterns of three contiguous atom arrangements are shown in the following diagram:

| | | | | |
|---|---|---|---|---|
| CCC | NCC | OCC | SCC | PCC |
| CCN | NCN | OCN | SCN | PCN |
| CCO | NCO | OCO | SCO | PCO |
| CCS | NCS | OCS | SCS | PCS |
| CCP | NCP | OCP | SCP | PCP |
| CNC | NNC | ONC | SNC | PNC |
| CNN | NNN | ONN | SNN | PNN |
| CNO | NNO | ONO | SNO | PNO |
| CNS | NNS | ONS | SNS | PNS |
| CNP | NNP | ONP | SNP | PNP |
| COC | NOC | OOC | SOC | POC |
| CON | NON | OON | SON | PON |
| COO | NOO | OOO | SOO | POO |
| COS | NOS | OOS | SOS | POS |
| COP | NOP | OOP | SOP | POP |
| CSC | NSC | OSC | SSC | PSC |
| CSN | NSN | OSN | SSN | PSN |
| CSO | NSO | OSO | SSO | PSO |
| CSS | NSS | OSS | SSS | PSS |
| CSP | NSP | OSP | SSP | PSP |
| CPC | NPC | OPC | SPC | PPC |
| CPN | NPN | OPN | SPN | PPN |
| CPO | NPO | OPO | SPO | PPO |
| CPS | NPS | OPS | SPS | PPS |
| CPP | NPP | OPP | SPP | PPP |

One skilled in the art would be able to identify bonding patterns that would produce multivalent compounds. Methods for producing these bonding arrangements are described in March, “Advanced Organic Chemistry”, 4th Edition, Wiley-Interscience, New York, N.Y. (1992). These arrangements are described in the grid of dots shown in the scheme above. All of the possible arrangements for the five most preferred atoms are shown. Each atom has a variety of acceptable oxidation states. The bonding arrangements underlined are less acceptable and are not preferred.

Examples of molecular structures in which the above bonding patterns could be employed as components of the linker are shown below.

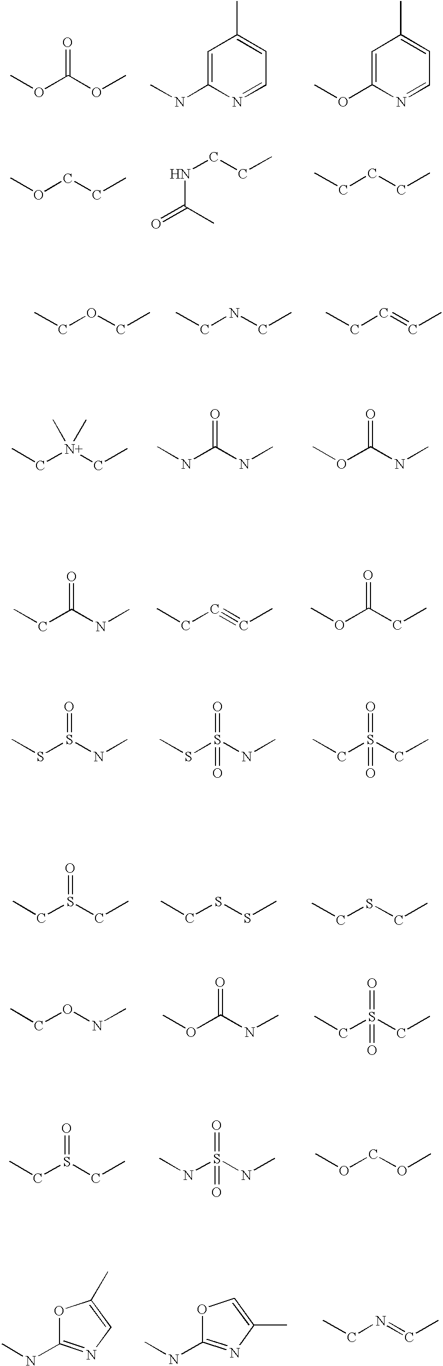

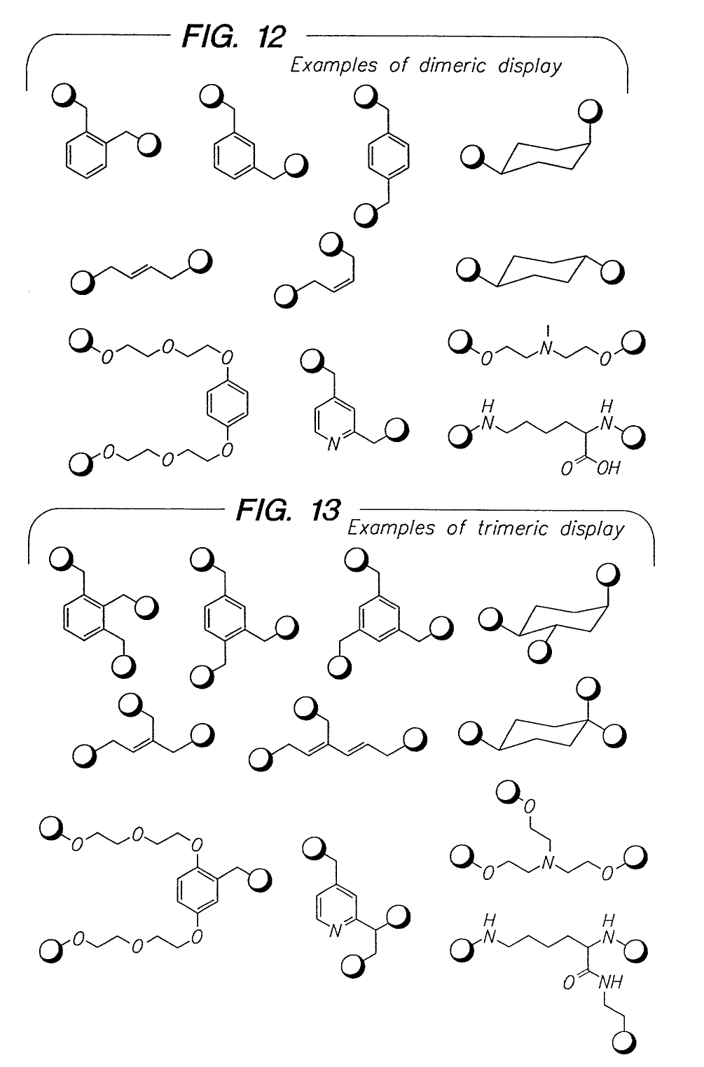

The identification of an appropriate framework geometry and size for macromolecular ligand do main presentation are important steps in the construction of a multibinding compound with enhanced activity. Systematic spatial searching strategies can be used to aid in the identification of preferred frameworks through an iterative process. FIG. 12 illustrates a useful strategy for determining an optimal framework display orientation for ligand domains. Various other strategies are known to those skilled in the art of molecular design and can be used for preparing compounds of this invention.

As shown in FIG. 12, display vectors around similar central core structures such as a phenyl structure and a cyclohexane structure cn be varied, as can the spacing of the ligand domain from the core structure (i.e., the length of the attaching moiety). It is to be noted that core structures other than those shown here can be used for determining the optimal framework display orientation of the macromolecular ligands. The process may require the use of multiple copies of the same central core structure or combinations of different types of display cores.

The above-described process can be extended to trimers (FIG. 13) and compounds of higher valency.

Assays of each of the individual compounds of a collection generated as described above will lead to a subset of compounds with the desired enhanced activities (e.g., potency, selectivity, etc.). The analysis of this subset using a technique such as Ensemble Molecular Dynamics will provide a framework orientation that favors the properties desired. A wide diversity of linkers is commercially available (see, e.g., Available Chemical Directory (ACD)). Many of the linkers that are suitable for use in this invention fall into this category. Other can be readily synthesized by methods well known in the art and/or are described below.

Having selected a preferred framework geometry, the physical properties of the linker can be optimized by varying the chemical composition thereof. The composition of the linker can be varied in numerous ways to achieve the desired physical properties for the multibinding compound.

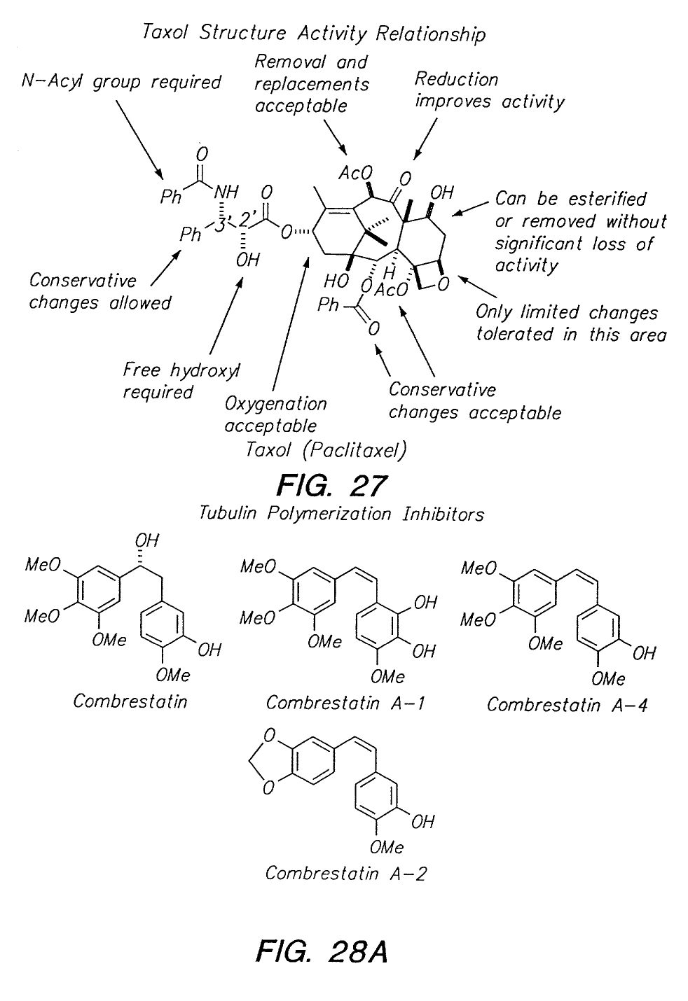

An example of this process for extending the framework from the ligand is presented in the attached figures for both taxol and amphotericin which examples can be readily extended to ligands for macromolecular structures using the same principles illustrated therein. Specifically, in the case of taxol, FIG. 27 illustrates some of the acceptable possible locations for elaboration into the framework.

Examples of multivalent amphotericin and taxol compounds are shown in the figures attached hereto.

It can therefore be seen that there is a plethora of possibilities for the composition of a linker. Examples of linkers include aliphatic moieties, aromatic moieties, steroidal moieties, peptides, and the like. Specific examples are peptides or polyamides, hydrocarbons, aromatic groups, ethers, lipids, cationic or anionic groups, or a combination thereof.

Examples are given below, but it should be understood that various changes may be made and equivalents may be substituted without departing from the true spirit and scope of the invention. For example, properties of the linker can be modified by the addition or insertion of ancillary groups into or onto the linker, for example, to change the solubility of the multibinding compound (in water, fats, lipids, biological fluids, etc.), hydrophobicity, hydrophilicity, linker flexibility, antigenicity, stability, and the like. For example, the introduction of one or more poly(ethylene glycol) (PEG) groups onto or into the linker enhances the hydrophilicity and water solubility of the multibinding compound, increases both molecular weight and molecular size and, depending on the nature of the unPEGylated linker, may increase the in vivo retention time. Further PEG may decrease antigenicity and potentially enhances the overall rigidity of the linker.

Ancillary groups which enhance the water solubility/hydrophilicity of the linker and, accordingly, the resulting multibinding compounds are useful in practicing this invention. Thus, it is within the scope of the present invention to use ancillary groups such as, for example, small repeating units of ethylene glycols, alcohols, polyols (e.g., glycerin, glycerol propoxylate, saccharides, including mono-, oligosaccharides, etc.), carboxylates (e.g., small repeating units of glutamic acid, acrylic acid, etc.), amines (e.g., tetraethylenepentamine), and the like) to enhance the water solubility and/or hydrophilicity of the multibinding compounds of this invention. In preferred embodiments, the ancillary group used to improve water solubility/hydrophilicity will be a polyether.

The incorporation of lipophilic ancillary groups within the structure of the linker to enhance the lipophilicity and/or hydrophobicity of the multibinding compounds described herein is also within the scope of this invention. Lipophilic groups useful with the linkers of this invention include, by way of example only, aryl and heteroaryl groups which, as above, may be either unsubstituted or substituted with other groups, but are at least substituted with a group which allows their covalent attachment to the linker. Other lipophilic groups useful with the linkers of this invention include fatty acid derivatives which do not form bilayers in aqueous medium until higher concentrations are reached.

Also within the scope of this invention is the use of ancillary groups which result in the multibinding compound being incorporated into a vesicle or other membranous structure such as a liposome or a micelle. The term “lipid” refers to any fatty acid derivative that is capable of forming a bilayer or a micelle such that a hydrophobic portion of the lipid material orients toward the bilayer while a hydrophilic portion orients toward the aqueous phase. Hydrophilic characteristics derive from the presence of phosphate, carboxylic, sulfato, amino, sulfhydryl, nitro and other like groups well known in the art. Hydrophobicity could be conferred by the inclusion of groups that include, but are not limited to, long chain saturated and unsaturated aliphatic hydrocarbon groups of up to 20 carbon atoms and such groups substituted by one or more aryl, heteroaryl, cycloalkyl, and/or heterocyclic group(s). Preferred lipids are phosphglycerides and sphingolipids, representative examples of which include phosphatidylcholine, phosphatidylethanolamine, phosphatidylserine, phosphatidylinositol, phosphatidic acid, palmitoyleoyl phosphatidylcholine, lysophosphatidylcholine, lysophosphatidyl-ethanolamine, dipalmitoylphosphatidylcholine, dioleoylphosphatidylcholine, distearoylphosphatidylcholine or dilinoleoylphosphatidylcholine could be used. Other compounds lacking phosphorus, such as sphingolipid and glycosphingolipid families are also within the group designated as lipid. Additionally, the amphipathic lipids described above may be mixed with other lipids including triglycerides and sterols.

The flexibility of the linker can be manipulated by the inclusion of ancillary groups which are bulky and/or rigid. The presence of bulky or rigid groups can hinder free rotation about bonds in the linker or bonds between the linker and the ancillary group(s) or bonds between the linker and the functional groups. Rigid groups can include, for example, those groups whose conformational lability is restrained by the presence of rings and/or multiple bonds within the group, for example, aryl, heteroaryl, cycloalkyl, cycloalkenyl, and heterocyclic groups. Other groups which can impart rigidity include polypeptide groups such as oligo- or polyproline chains.

Rigidity can also be imparted electrostatically. Thus, if the ancillary groups are either positively or negatively charged, the similarly charged ancillary groups will force the presenter linker into a configuration affording the maximum distance between each of the like charges. The energetic cost of bringing the like-charged groups closer to each other will tend to hold the linker in a configuration that maintains the separation between the like-charged ancillary groups. Further ancillary groups bearing opposite charges will tend to be attracted to their oppositely charged counterparts and potentially may enter into both inter- and intramolecular ionic bonds. This non-covalent mechanism will tend to hold the linker into a conformation which allows bonding between the oppositely charged groups. The addition of ancillary groups which are charged, or alternatively, bear a latent charge when deprotected, following addition to the linker, include deprotectation of a carboxyl, hydroxyl, thiol or amino group by a change in pH, oxidation, reduction or other mechanisms known to those skilled in the art which result in removal of the protecting group, is within the scope of this invention.

Rigidity may also be imparted by internal hydrogen bonding or by hydrophobic collapse.

Bulky groups can include, for example, large atoms, ions (e.g., iodine, sulfur, metal ions, etc.) or groups containing large atoms, polycyclic groups, including aromatic groups, non-aromatic groups and structures incorporating one or more carbon-carbon multiple bonds (i.e., alkenes and alkynes). Bulky groups can also include oligomers and polymers which are branched- or straight-chain species. Species that are branched are expected to increase the rigidity of the structure more per unit molecular weight gain than are straight-chain species.

In preferred embodiments, rigidity is imparted by the presence of cyclic groups (e.g., aryl, heteroaryl, cycloalkyl, heterocyclic, etc.). In other preferred embodiments, the linker comprises one or more six-membered rings. In still further preferred embodiments, the ring is an aryl group such as, for example, phenyl or naphthyl.

In view of the above, it is apparent that the appropriate selection of a linker group providing suitable orientation, restricted/unrestricted rotation, the desired degree of hydrophobicity/hydrophilicity, etc. is well within the skill of the art. Eliminating or reducing antigenicity of the multibinding compounds described herein is also within the scope of this invention. In certain cases, the antigenicity of a multibinding compound may be eliminated or reduced by use of groups such as, for example, poly(ethylene glycol).

As explained above, the multibinding compounds described herein comprise 2-10 macromolecular ligands attached to a linker that links the ligands in such a manner that they are presented to the macromolecular structure for multivalent interactions with ligand binding sites thereon/therein. The linker spatially constrains these interactions to occur within dimesions defined by the linker. This and other factors increases the biological activity of the multibinding compound as compared to the same number of ligands made available in monobinding form.

The compounds of this invention are preferably represented by the empirical formula (L)ₚ(X)_{q} where L, X, p and q are as defined above. This is intended to include the several ways in which the ligands can be linked together in order to achieve the objective of multivalency, and a more detailed explanation is described below.

As noted previously, the linker may be considered as a framework to which ligands are attached. Thus, it should be recognized that the ligands can be attached at any suitable position on this framework, for example, at the termini of a linear chain or at any intermediate position.

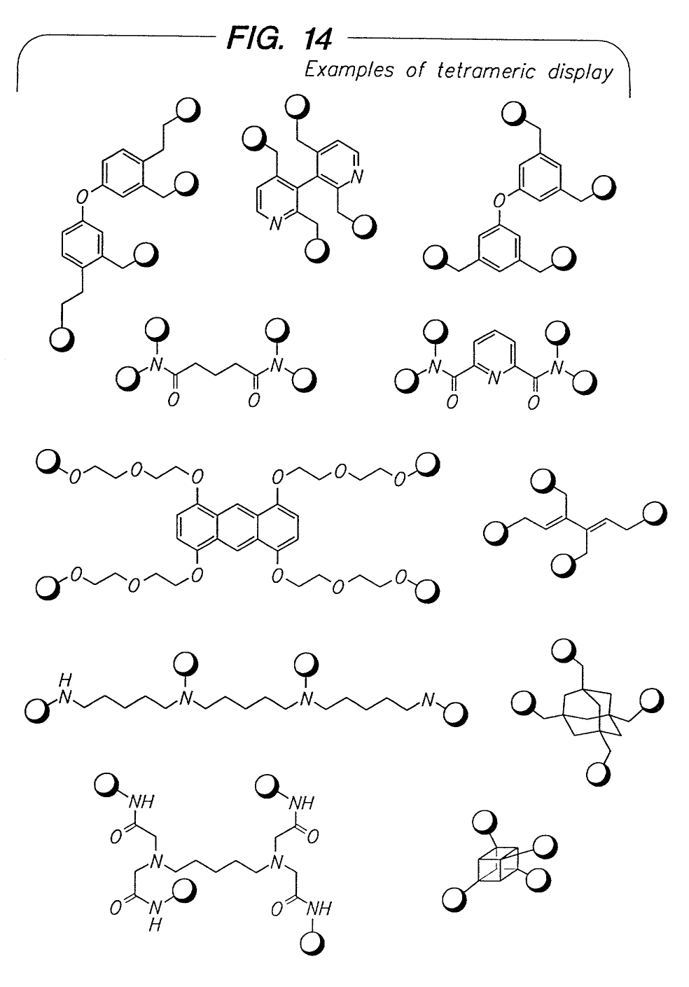

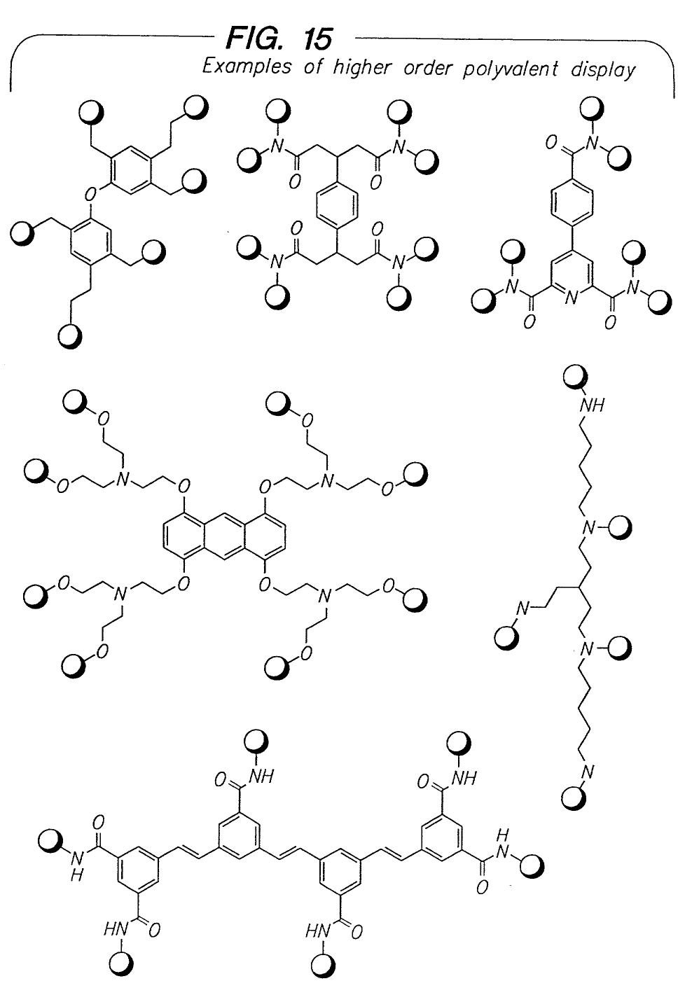

The simplest and most preferred multibinding compound is a bivalent compound which can be represented as L—X—L, where each L is independently a ligand which may be the same or different and each X is independently the linker. Examples of such bivalent compounds are provided in FIG. 12 where each shaded circle represents a ligand. A trivalent compound could also be represented in a linear fashion, i.e., as a sequence of repeated units L—X—L—X—L, in which L is a ligand and is the same or different at each occurrence, as can X. However, a trimer can also be a radial multibinding compound comprising three ligands attached to a central core, and thus represented as (L)₃X, where the linker X could include, for example, an aryl or cycloalkyl group. Illustrations of trivalent and tetravalent compounds of this invention are found in FIGS. 13 and 14 respectively where, again, the shaded circles represent ligands. Tetravalent compounds can be represented in a linear array, e.g.,

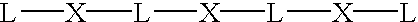

in a branched array, e.g.,

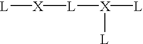

(a branched construct analogous to the isomers of butane—n-butyl, iso-butyl, sec-butyl, and t-butyl) or in a tetrahedral array, e.g.,

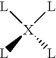

where X and L are as defined herein. Alternatively, it could be represented as an alkyl, aryl or cycloalkyl derivative as above with four (4) ligands attached to the core linker.

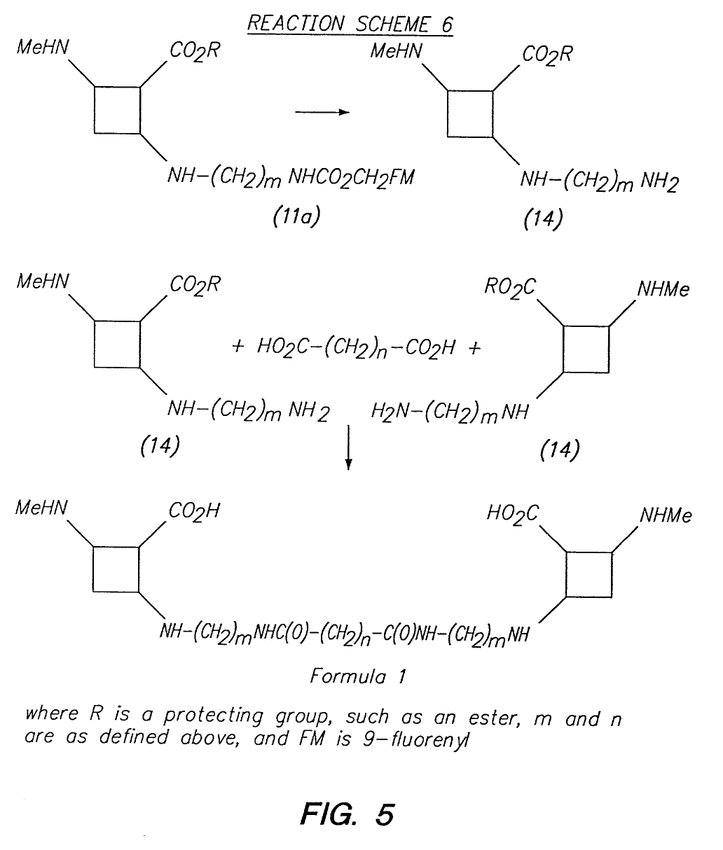

The same considerations apply to higher multibinding compounds of this invention containing 5-10 macromolecular ligands as illustrated in FIG. 5 where, as before, the shaded circles represent ligands. However, for multibinding agents attached to a central linker such as aryl or cycloalkyl, there is a self-evident constraint that there must be sufficient attachment sites on the linker to accommodate the number of ligands present; for example, a benzene ring could not directly accommodate more than 6 ligands, whereas a multi-ring linker (e.g., biphenyl) could accommodate a larger number of ligands.

Certain of the above described compounds may alternatively be represented as cyclic chains of the form:

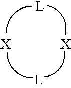

and variants thereof.

All of the above variations are intended to be within the scope of the invention defined by the formula (L)ₚ(X)_{q}.

With the foregoing in mind, a preferred linker may be represented by the following formula:

—X′—Z—(Y′—Z)ₘ—Y″—Z—X′—

in which:

m is an integer of from 0 to 20;

X′ at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;

Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;

Y′ and Y″ at each separate occurrence are selected from the group consisting of:

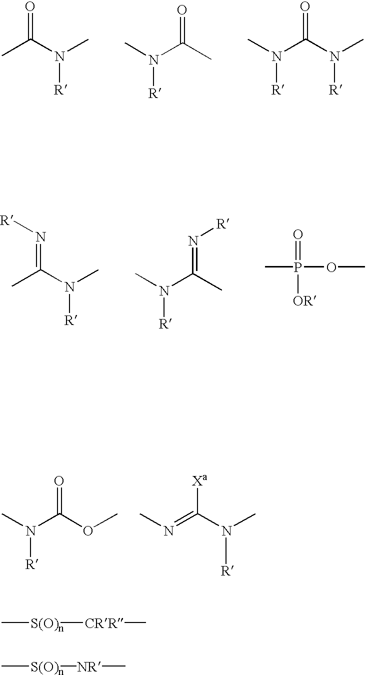

—S—S— or a covalent bond;

in which:

n is 0, 1 or 2; and

R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

Additionally, the linker moiety can be optionally substituted at any atom therein by one or more alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic group.

In view of the above, it is understood that the term “linker” when used in combination with the term “multibinding compound” includes both a covalently contiguous single linker (e.g., L—X—L) and multiple covalently non-contiguous linkers (L—X—L—X—L) within the multibinding compound. In addition, when the linker contains a chiral center, the linker may be a racemic mixture or a stereochemically pure compound.

Preparation of Multibinding Compounds

Accordingly, multibinding compounds of this invention may be prepared as shown as below.

As indicated above, the simplest (and preferred) multibinding compound is a bivalent compound, which can be represented L—X—L, where each L is independently a ligand which may be the same or different at each occurrence, and each X is independently the linker.

An example of the preparation of a bivalent macromolecular structure ligand is given below as an illustration of the manner in which multibinding agents of this invention are obtained. This example is applicable to any macromolecular ligand that includes amino and/or carboxyl groups, for example, amphotericin, polymyxin, and similar ligands known to bind macromolecular structures. Examples of different linkers X are shown. In the reaction schemes that follow, for ease of understanding of the principles involved, the structure of ligand is represented as a “box”. Thus, the ligand is illustrated such that carboxyl [C], amino [V], and methylamino [N] groups are shown as examples of connecting points for the linker. However, as before, the ligand employed is one which binds to macromolecular structures.

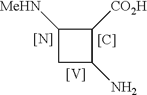

Two macromolecular ligands are connected by the linker X via carboxyl group or amino group of a first ligand to any carboxyl group or amino group of a second ligand.

Another simplification in the description of the preparations is that, for example, compound (1) is illustrated as a compound of formula H₂N—(CH₂)ₘ—NCHO₂-t-butyl, in which m is an integer of 1-20. However, it should be understood that (CH₂)ₘ is not intended to signify or imply that the scope of this reaction (or of the invention) is limited to straight (i.e. unbranched) alkylene chains, but rather (CH₂)ₘ is intended to include branched alkylenes as defined in above, substituted alkylenes, and the like, also as disclosed above. Similarly, the compound of formula (2) is illustrated as ClC(O)—(CH₂)ₙ—C(O)Cl, and (CH₂)ₙ equally is not limited to straight alkylene chains, but includes all those modifications shown above.

Accordingly, bivalent compounds of Formula I where the linkage is from a [C] group of a first ligand to a [C] group of a second ligand, i.e. a [C—C] linkage, may be prepared from intermediates of formula (4), the preparation of which is shown below in Reaction Scheme 1.

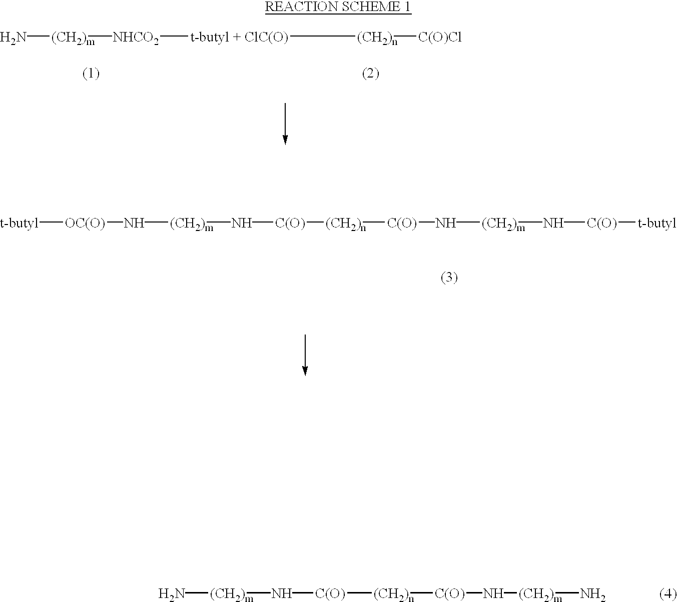

in which m and n are independently, at each occurrence, intergers of 1-20.

Preparation of Compounds of Formula (3)

As illustrated in Reaction Scheme 1, step 1, about two molar equivalents of an omega-amino carbamic acid ester [compound (1)] is reacted with about one molar equivalent of a dicarboxylic acid halide, preferably chloride, of formula (2). The reaction is preferably conducted in the presence of a non-nucleophilic base (e.g., a hindered base), preferably duisopropylethylamine, in order to scavenge the acid generated during reaction. The reaction is preferably conducted in an inert solvent, preferably methylene chloride, at a temperature of about 0˜5° C. The mixture is then allowed to warm to room temperature. When the reaction is substantially complete, the compound of formula (3) is isolated and purified by conventional means.

Preparation of Compounds of Formula (4)

As illustrated in Reaction Scheme 1, step 2, t-Boc protecting groups of carbamate (3) are removed under acidic conditions. In general, a preferred acid is trifluoroacetic acid, and the reaction is conducted in an inert solvent, preferably methylene chloride, at about room temperature. When the reaction is substantially complete, the compound of formula (4) is isolated and purified by conventional means.

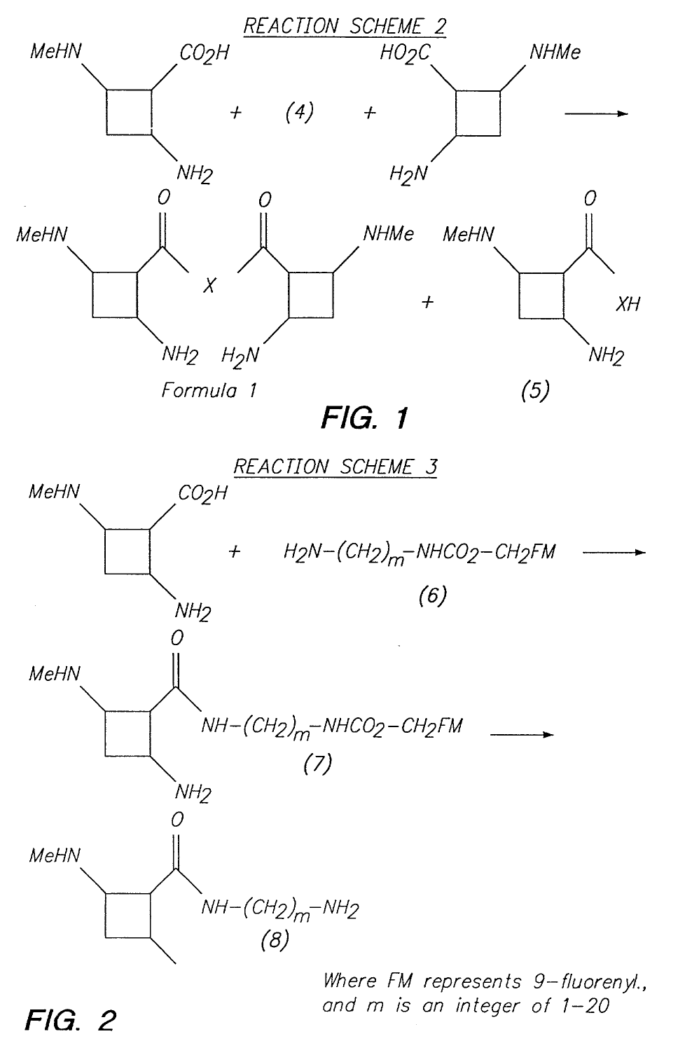

The compound of formula (4) is then converted into a [C—C] ligand dimer as shown in Reaction Scheme 2 illustrated in FIG. 1.

### Reaction Scheme 2

Preparation of Compounds of Formula I

In Reaction Scheme 2, the ligand for the macromolecular structure is depicted as a box having primary amino, a secondary amino and carboxyl functionality.

In general, about two molar equivalents of ligand is reacted with about one molar equivalent of the compound of formula (4), under conventional amide coupling conditions. Preferably, a hindered base is employed to scavenge the acid generated, preferably diisopropylethylamine, in the presence of coupling reagents such as benzotriazol-1-yloxytripyrrolidinophosphonium hexafluorophosphate (PyBOP) and 1-hydroybenzotriazole. The reaction is conducted in an inert polar solvent, for example, N,N-dimethylformamide (DMF) or dimethyl sulfoxide (DMSO), or preferably a mixture of both, at about room temperature. When the reaction is substantially complete, the compound of Formula I is isolated and purified by conventional means, preferably purified by reverse phase HPLC. Also isolated is a byproduct of formula (5).

Alternatively, compounds of Formula I [C—C] may be prepared from intermediates of formula (8), the preparation of which is shown below in Reaction Scheme 3 illustrated in FIG. 2.

### Reaction Scheme 3

Preparation of Compounds of Formula (7)

As illustrated in Reaction Scheme 3, step 1, ligand is reacted with about 1.1 molar equivalents of a carbamic ester terminated by an alkylamino group [compound (6)]. The ester moiety is chosen for ease of removal under mild conditions in subsequent reactions, and is preferably 9-fluorenylmethyl (FM). Conventional amide coupling conditions are employed, preferably using PyBOP and 1-hydroxybenzotriazole. In general, the reaction is conducted in the presence of a hindered base, preferably diisopropylethylamine, to scavenge the acid generated and the reaction is conducted in an inert polar solvent, preferably DMF or DMSO, preferably a mixture of both, at about room temperature. When the reaction is substantially complete, the compound of formula (7) is isolated and purified by conventional means.

Preparation of Compounds of Formula (8)

As illustrated in Reaction Scheme 3, step 2, the compound of formula (7) is reacted with a mild base to remove the protecting ester groups, which also affords decarboxylation. In general, the base is preferably piperidine, and the reaction is conducted in an inert polar solvent, preferably dimethylformamide, at about room temperature for about 10 minutes to one hour. When the reaction is substantially complete, the compound of formula (8) is isolated and purified by conventional means, preferably using reverse phase HPLC.

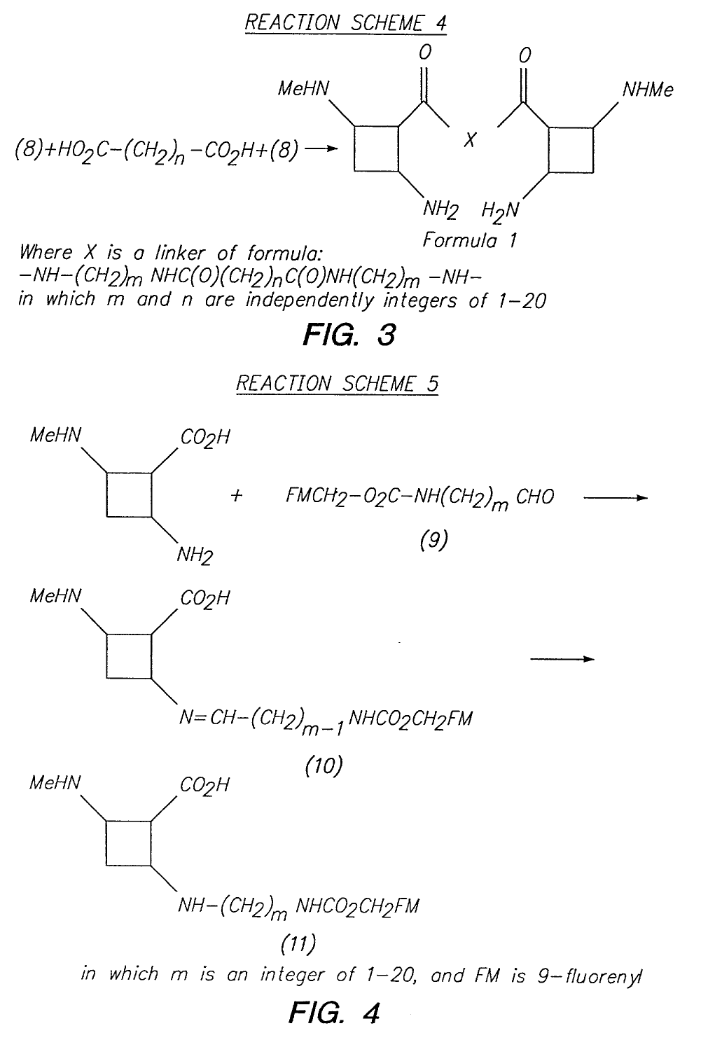

The compound of formula (8) is then converted into a [C—C] ligand dimer as shown in Reaction Scheme 4 found in FIG. 3.

### Reaction Scheme 4

Preparation of Compounds of Formula I

As illustrated in Reaction Scheme 4, the compound of formula (8) is reacted with a dicarboxylic acid. In general, about 3 molar equivalents of the compound of formula (8) is reacted with about 1 molar equivalent of the dicarboxylic acid of formula HO₂C—(CH₂)ₙ—CO₂H, under conventional amide coupling conditions. Preferably, a hindered base is employed, preferably diisopropylethylamine, in the presence of PyBOP and 1-hydroxybenzotriazole. The reaction is conducted in an inert polar solvent, preferably DMF, at about room temperature for about 1-3 hours. When the reaction is substantially complete, the compound of Formula I is isolated and purified by conventional means, preferably purified by a reverse phase of HPLC.

Compounds of Formula I wherein the linkage is [V—V] may be prepared from intermediates of formula (14), the preparation of which is shown below in Reaction Scheme 6 found in FIG. 5. The starting material, the compound of formula (11), is prepared as shown in Reaction Scheme 5 found in FIG. 4.

### Reaction Scheme 5

Preparation of Compounds of Formula (10)

As illustrated in Reaction Scheme 5, step 1, ligand having an —NH₂ group suitable for linking is reacted with a protected ester-aldehyde of formula (9) to form a Schiff's base. The ester moiety is chosen for ease of removal under mild conditions in subsequent reactions, and is preferably 9-fluorenylmethyl. In general, the reaction is conducted in an inert polar solvent, preferably 3-dimethyl-3,4,5,6-tetrahydro˜2(1H)-pyrimidinone plus methanol, at about 25° to about 100° C., until the reaction is complete. The Schiff's base of formula (10) is not isolated, but reacted further immediately as shown below.

Preparation of Compounds of Formula (11)

As illustrated in Reaction Scheme 5, step 2, the solution of the compound of formula (10) is further reacted with a mild reducing agent (not shown). In general, the reducing agent is preferably sodium cyanoborohydride, and the reaction is conducted at about 25° to about 100° C., preferably about 70° C., for about 1-3 hours, preferably about 2 hours. When the reaction is substantially complete, the compound of formula (11) is isolated and purified by conventional means, preferably purified by reverse phase HPLC.

Compounds of Formula I wherein the linkage is [V—V] may then be prepared from intermediates of formula (11a), the preparation of which is shown below in Reaction Scheme 6 found in FIG. 5.

### Reaction Scheme 6

Preparation of Compounds of Formula (14)

As illustrated in Reaction Scheme 6, step 1, the compound of formula (11a), which is a compound of formula (11) in which the carboxyl group has been protected conventionally, for example as an ester (indicated by R), is reacted with a mild base to remove the carbamate. In general, the base is preferably piperidine, and the reaction is conducted in an inert polar solvent, preferably DMF, at about room temperature for about 10 minutes to one hour, preferably about 30 minutes. When the reaction is substantially complete, the compound of formula (14) is isolated and purified by conventional means, preferably using reverse phase HPLC.

Preparation of Compounds of Formula I

As illustrated in Reaction Scheme 6, step 2, the compound of formula (14) is reacted with a dicarboxylic acid. In general, about 3 molar equivalents of the compound of formula (8) is reacted with about 1 molar equivalent of the dicarboxylic acid of formula HO₂C—(CH₂)ₙ—CO₂H, under conventional amide coupling conditions. Preferably, a hindered base is employed, preferably diisopropylethylamine, in the presence of PyBOP and 1-hydroxybenzotriazole. The reaction is conducted in an inert polar solvent, preferably DMF, at about room temperature for about 1-3 hours. When the reaction is substantially complete, the protecting group R, preferably and ester, is removed conventionally, and the [V—V] compound of Formula I is isolated and purified by conventional means, preferably purified by reverse phase HPLC.

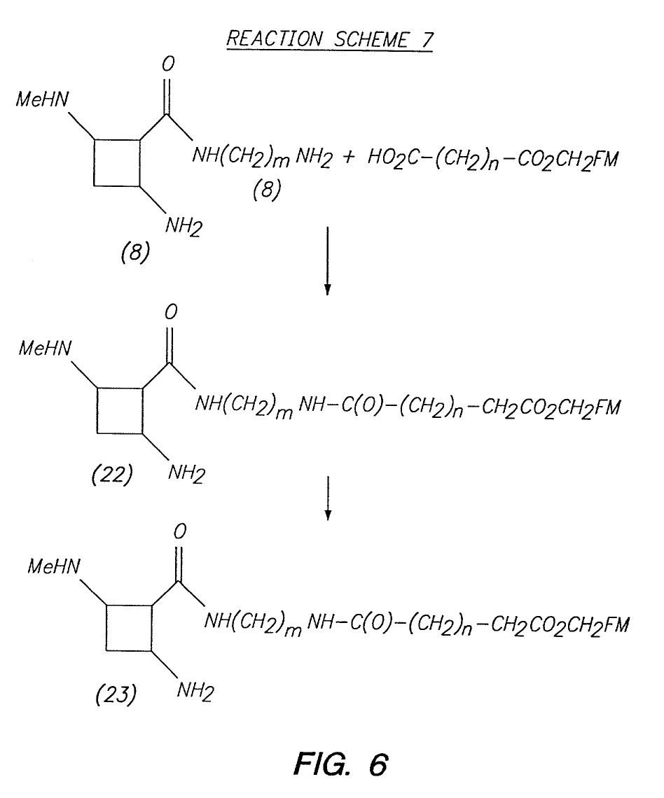

Compounds of Formula I wherein the linkage is [C—V] may be prepared from intermediates of formula (23), the preparation of which is shown below in Reaction Scheme 7 found in FIG. 6. The starting material, the compound of formula (8), is prepared as previously shown.

### Reaction Scheme 7

Preparation of Compounds of Formula (22)

As illustrated in Reaction Scheme 7, step 1, the compound of formula (8) is reacted with an acid in the same manner as shown above to form an amide of formula (22).

Preparation of Compounds of Formula (23)

As illustrated in Reaction Scheme 7, step 2, the compound of formula (22) is hydrolyzed in the same manner as shown above to form a compound of formula (23).

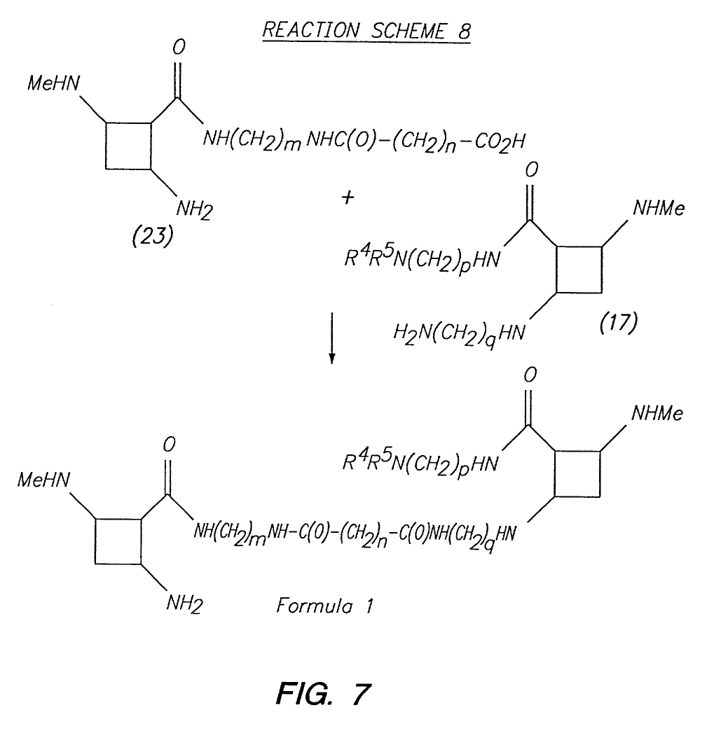

The compound of formula (23) is then converted into a [C—V] dimer of Formula I by reaction with a compound of formula (17) as shown in Reaction Scheme 8 found in FIG. 7.

### Reaction Scheme 8

Preparation of Compounds of Formula I

As illustrated in Reaction Scheme 8, the compound of formula (23) is reacted with a compound of formula (17) in a conventional coupling reaction, as shown above, to give a compound of Formula I [C—V].

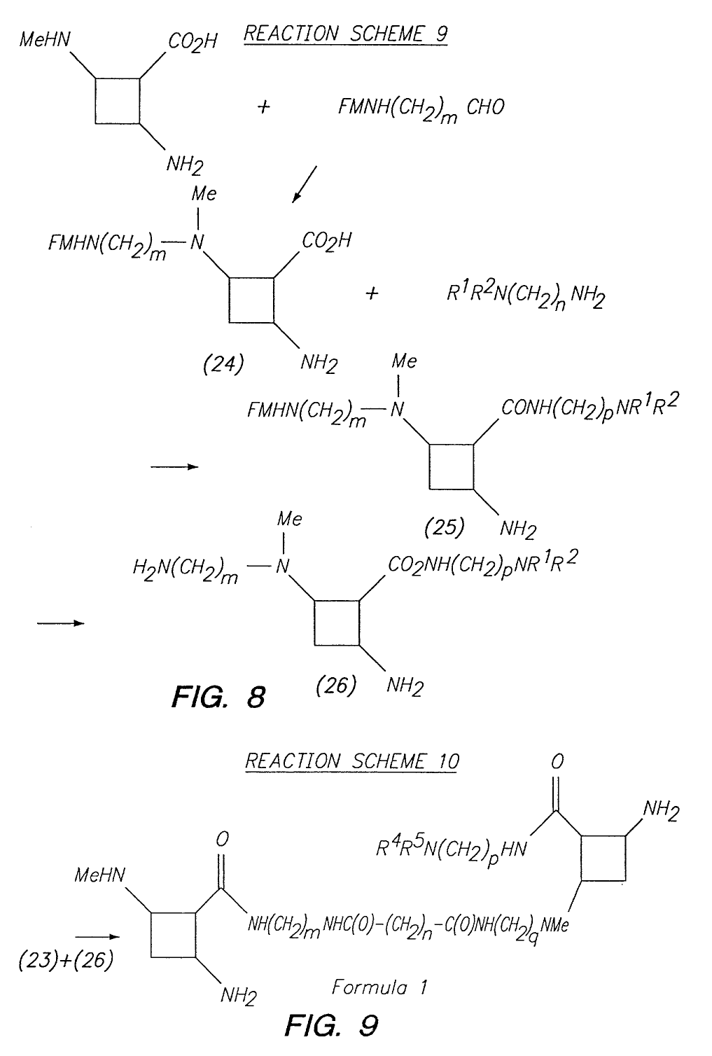

Compounds of Formula I wherein linkage is [C—N] may be prepared from intermediates of formula (26), the preparation of which is shown below in Reaction Scheme 9 found in FIG. 8.

### Reaction Scheme 9

Preparation of Compounds of Formula (24)

As illustrated in Reaction Scheme 9, step 1, ligand is reacted with a protected aminoaldehyde in the presence of an amount of base sufficient to direct the reaction of the aldehyde to the [N] position. The Schiff s base thus formed is reduced in the same manner as shown in Reaction Scheme 5 to form a compound of formula (24).

Preparation of Compounds of Formula (25)

As illustrated in Reaction Scheme 9, step 2, the compound of formula (24) is reacted with an amine in a coupling reaction in the same manner as shown above, for example in Reaction Scheme 10, to form an amide of formula (25).

Preparation of Compounds of Formula (26)

As illustrated in Reaction Scheme 9, step 3, the protecting group FM is removed conventionally from the compound of formula (25) with a mild base to form a compound of formula (26).

The compound of formula (26) is then converted into a [C—N] dimer of Formula I by reaction with a compound of formula (23), prepared as shown previously, as shown in Reaction Scheme 10 found in FIG. 9.

### Reaction Scheme 10

Preparation of Compounds of Formula I

As illustrated in Reaction Scheme 10, the compound of formula (26) is reacted with a compound of formula (23) in a typical coupling reaction as shown above, for example in Reaction Scheme 11, to give a compound of Formula I [C—N].

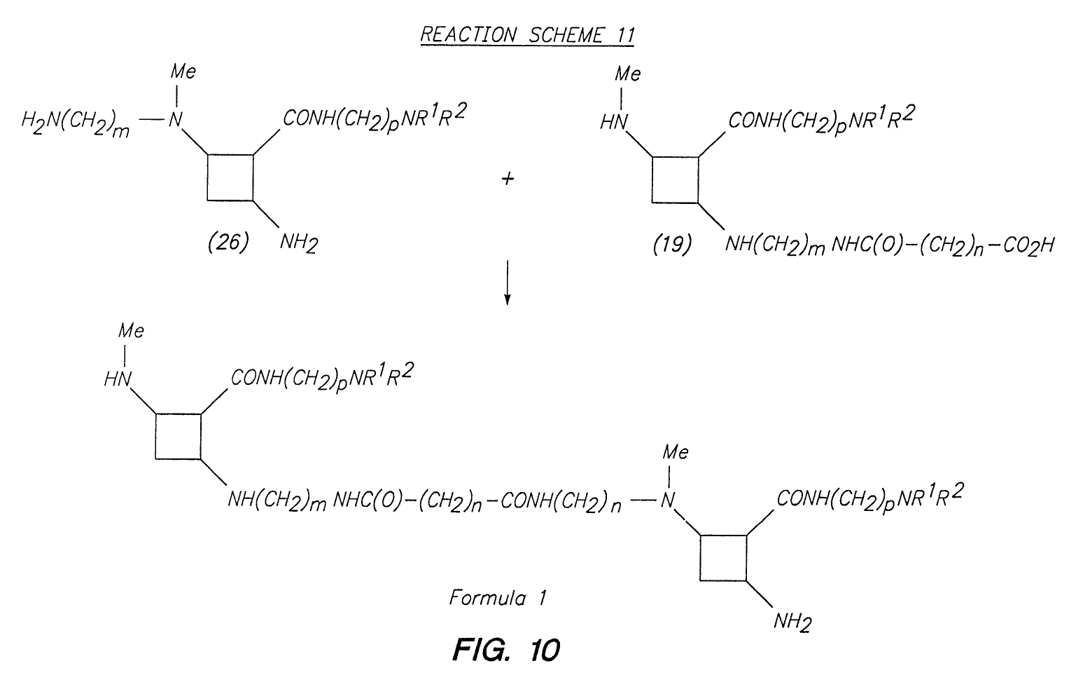

Compounds of Formula I wherein the linkage is [N—V] may be prepared by a reaction of a compound of formula (26) with a compound of formula (19), as shown in Reaction Scheme 11 found in FIG. 10.

### Reaction Scheme 11

Preparation of Compounds of Formula I

As illustrated in Reaction Scheme 11, the compound of formula (26) is reacted with a compound of formula (19) in a conventional coupling reaction as shown above, to give a compound of Formula I [N—V].

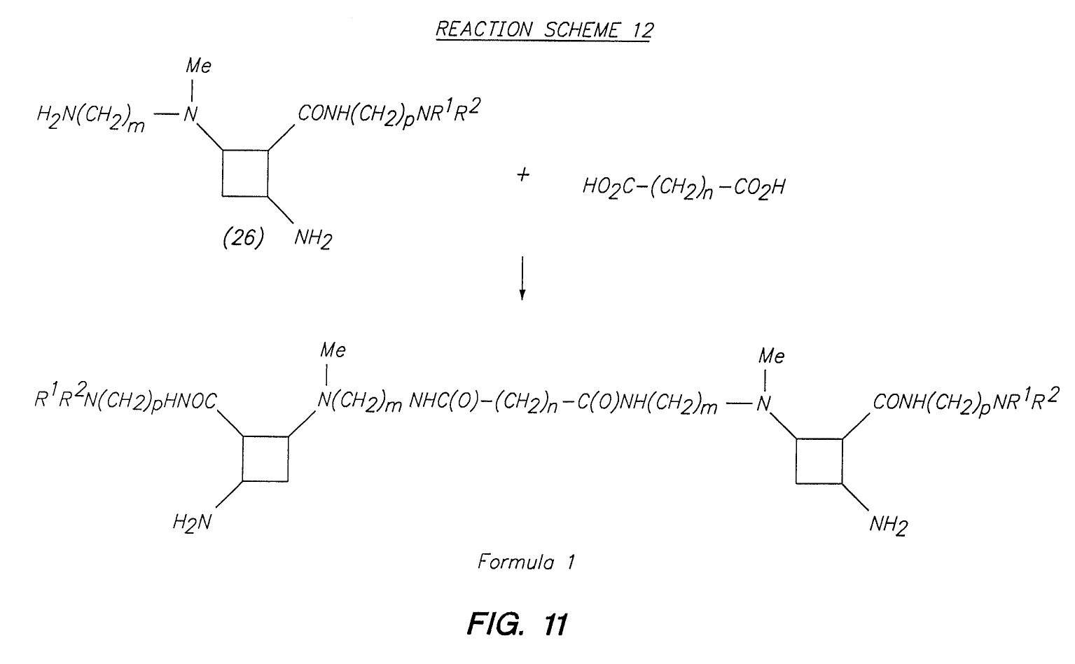

Compounds of Formula I wherein the linkage is [N—N] may be prepared by reaction of a compound formula (26) with a dicarboxylic acid of formula HO₂C—(CH₂)ₙ—CO₂H, as shown in Reaction Scheme 12 found in FIG. 11.

### Reaction Scheme 12

Preparation of Compounds of Formula I

As illustrated in Reaction Scheme 12, the compound of formula (26) is reacted with a dicarboxylic acid of formula HO₂C—(CH₂)ₙ—CO₂H in the same manner as shown above in Reaction Scheme 4, to give a compound of Formula I [N—N].

It should be noted that ligands having terminal amino groups may also be linked by reaction with aldehyde linkers (to form a Schiff's base, that can be used as a linker itself or preferably reduced with, e.g., sodium cyanoborohydride, to an amine linker). Alternatively, reaction with a disulfonyl halide would give a sulfonamide linker. Another alternative synthesis encompasses reaction of an amine with a diiminoester which would afford an amidine linker.

Ligands that include a free hydroxy group in their structure (an alcohol or phenolic hydroxy) may be connected using those hydroxy groups as linkage points by means well known in the art. For example, one synthetic strategy that could be used for linking ligands with free hydroxy groups involves treating the ligand with t-butyl bromoacetate in the presence of a base (e.g. potassium carbonate) to convert the —OH group to an —O—CH₂CO₂-t-But group, which can be deprotected/converted to an —O—CH₂CO₂H group using trifluoracetic acid. The oxyacetic group can then be used as the linking point for two ligands by making use of the linking strategies shown above for carboxylic acids. For example, reaction of two molar equivalents of the ligand with a diamine of the formula H₂N—(CH₂)ₙ—NH₂, where n is an integer of 1-20, leads to two ligands being connected by a linker of the formula —CH₂CONH—(CH₂)ₙ—NHCOCH₂—.

Alternatively, treating the hydroxy-bearing ligand with BOC—NHCH₂CH₂Br in the presence of a base (e.g. potassium carbonate) converts the —OH group to an O—CH₂CH₂NHBOC group, which can be deprotected to an —O—CH₂CH₂NH₂ group using trifluoracetic acid. The oxyethylamino group can then be used as the linking point for two ligands by making use of the linking strategies shown above for amines. For example, reaction of two molar equivalents of the ligand with a dicarboxylic acid of the formula HO₂C—(CH₂)ₙ—CO₂H where n is an integer of 1-20, leads to two ligands being connected by a linker of the formula —CH₂ CH₂NHCO—(CH₂)ₙ—CONHCH₂CH₂—.

Alternatively, converting the hydroxy-group to a leaving group, for example, by treatment with mesyl chloride or tosyl chloride, or converting the hydroxy group to a halide by means well known in the art, the ligand can then be linked directly by reaction with a diamine.

The Mannich reaction can be used to link ligands that have an “active” hydrogen in their molecular structure. Examples of such active hydrogens are hydrogens that are adjacent to an electron withdrawing group such as a ketone, aldehyde, acid or ester, nitrile, nitro group and the like. The Mannich reaction is well known to those skilled in the art, and many reviews in the chemical literature and text books on the Mannich reaction are available. Of particular value is that a linker can be constructed on a ligand having an aromatic moiety, providing there is an active hydrogen.

In each of the reaction schemes set forth above, the use of conventional blocking groups can be used to further direct the reaction to the desired position. For example, compound 8 can be orthogonally protected at the two amines with differentially removable protecting groups, e.g., protecting groups which are removed under conditions such that removal of one group will not remove the other group. In such cases, the differentially removable groups can result in selective removal of one of the protecting groups thereby permitting subsequent conversion at that position without concern of competing reactions at the other still protected amino group.

In view of the above, the chemistry for attaching ligands to linkers is well established in the art and is well within the skill of the art.

The following examples are employed to specifically illustrate the attachment of several ligands to linkers as per this invention and the specific ligands employed are employed only for illustrative purposes and should not be construed as a limitation for this application.

Picornavirus Inhibitors

The family of picornaviruses include a number of important human and animal pathogens such as poliovirus, rhinoviruses (the main causative agent for the common cold), hepatitis A, the echo viruses which cause viral menengitis, and foot-and-mouth disease.¹ The members of this family share a common icosahedral structure but have been separated into five sub-classes based on organization of their respective genomes: enteroviruses, rhinoviruses, hepatoviruses, aphtoviruses, and cardioviruses. A typical viron particle contains 60 copies of four structural proteins VP1, VP2, VP3 and VP4, plus a copy of genomic RNA (approximately 7.5-8.5 kBP) and a genome bound viral protein VPg. These viral protein building blocks VP1-3, although varying in sequence, all share a similar tertiary structure comprised of two 4-stranded beta-sheets. Each building block resembles a trapeziodal solid. The virons pack in a spherical shape with icosahedral symmetry, each face of the icosahedron presenting a surface formed by the VP1, VP2, and VP3 building blocks. The amino termini of these three units intertwine to form a link in the interior of the viron; 5 of the VP3 amino terminii further form a 5-stranded, helical beta-cylinder in the interior to which myristoylated VP4 is associated. On the surface of each face of the capsid protein (with the exception of aphtopviruses) there is a depression or “canyon”, approximately 25 Å deep. This canyon region is associated with host cell surface receptor binding function (ICAM-I or other glycopeptide—IgG-like—receptors). Located below the floor of this canyon, within the VP1 beta-barrel, is a pore leading into a hydrophobic pocket. This pocket site has been identified by X-ray analysis of drug-virus complexes as the binding site for many small molecule inhibitors of the picornavirus infection process.² Very large numbers of drug molecules can bind to a viron particle as evidenced by X-ray or other data. Stoichiometries of >40:1 drug to viron are not uncommon consistent with the multiple symmetry related to the hydrophobic pockets.

Structural analysis of the drug-virus interaction indicates the drugs may disrupt the process of viral infection by rigidification of the protein. This would either prevent receptor binding, viral (protein) uncoating and release of the viral genome, membrane fusion, or a combination of these events.³ It is reasonable to assume that the mode of action of these drugs may result from a combination of these events depending on the affinity of drug binding and the relative depth and orientation of the drug within this binding pocket.

No drug that is known to directly inhibit uncapping of the capsid proteins has been approved for antiviral therapy. The slow development of compounds in this area is at least in part linked to the lack of drug potency. Thus greater affinity of compounds for the viron hydrophobic pocket will demonstrate more potent antiviral activity both in vitro and in vivo. The extensive metabolism, poor bioavailability at relevant tissue sites, and short half life of the compounds explored also appear to have limited the efficacy of compounds clinically investigated. The successful development of VP 63843 (pleconaril) to its current Phase III status was driven primary by circumventing the metabolism issues associated with earlier candidates⁴. Thus, pleconaril is a good starting point for designing a more potent drug.

Drug resistant piconavirus strains are known. These strains have reduced affinity for monomeric ligands in the pore. For example, genetic analysis of rhinovirus strains indicate that a significant mutation Val-Leu/Ile in the pore is likely responsible for a reduction in drug in vitro resistance.

Drug (Ligand) Structures

Some of the drugs that have been evaluated clinically are compounds A, B (pleconaril), C and D (pirodavirl)⁴. Compound A demonstrated a prophylactic effect in clinical trials (p.o. administration) with patients challenged with coxsackievirus A21. However, the compound did not demonstrate a therapeutic effect when administered orally to patients infected with HRV (human rhinovirus)-16, 39, and 50. Subsequently, the later generation compound B evolved which is metabolically much more stable. This compound is now in phase IIb, after successful completion of Phase II for viral meningitis and ‘summer flu’ (enteroviral respiratory syndrome). Compounds C and D also received clinical exploration. Clinical efficacy in the treatment of the common cold was first demonstrated by pirodavir (compound D) and compound C worked prophylactically but not when used therapeutically against HRV-9. Thus it appears that if PK/metabolism issues are addressed in the preclinical development of the antiviral agent and broad antiviral activity is demonstrated in vitro, it is reasonable to expect the agent to demonstrate clinical efficacy.

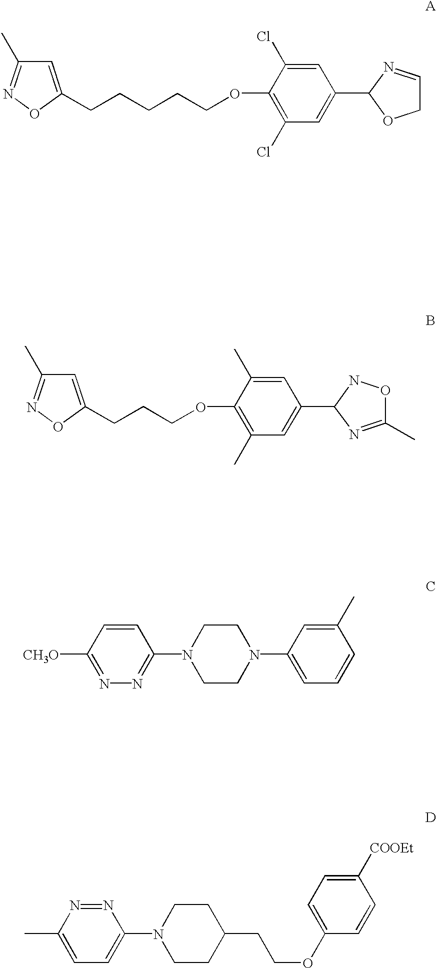

Multimeric drug complexes can be obtained by exploiting the relative C2, C3, or C5-symmetry of the isocosahedral picornavirons. The canyons on the surfaces of adjacent VP1-VP2-VP3 protomers are replicated such that dimeric, trimeric, or pentameric derivatives of plecornaril will form stabilized multimeric interactions with the viron to completely abrogate binding to cellular receptors. This mode of binding would not only ensure the stabilization of the capsid protein, eliminating its ability to uncoat, but, upstream of this event, sterically block the canyon floor and walls. This would prevent host cell receptors from accessing these critical binding regions of the viron. The preferred mode of binding would be to capture 2-5 canyon pores using a shorter rather than longer, linker between drug monomers. The distance between adjacent pores is expected to be less than 100 Å and more likely 15 to 50 Å. (The diameter of a picornavirus is approx 250 Å). Accordingly, the construction of three or five-centered star shaped linkers can be made such that at each terminus a drug monomer resides with the appropriate or optimal orientation for binding.

There are over 100 serotypes of RHV alone and representative compounds in the past have demonstrated over a 100 fold variation in minimum inhibitory activity (MIC) depending upon the subtype used. An added advantage that a multibinding antiviral compound provides is an increase in potency that leads to broader coverage of picornavirus subtype activity and a reduced susceptability to resistance. Although a monomer ligand may lose some affinity for a virion due to a mutation in the ligand binding site, a multibinding compound constructed with multiple members of the same ligand regains the affinity for the viron, thus circumventing the resistance. Additionally, heteromeric constructs with variant monomers could address both non-resistant and resistant strains by maintaining high affinity to all strains via monomer subtype selectivity.

X-ray crystal structure and computational analysis of pleconaril derivatives bound to rhinoviruses indicate that isosteres of the methyl isoxadiazole ring are situated deep into the hydrophobic pore on the floor of the virion cavity.⁵ This places the methylisoxazole ring near the pore opening. The exposed protein surface of the capsid pore opening is more hydrophilic relative to the internal pocket. Consistent with this observation is that a number of structural modifications where R is equal to methylene alcohols, methylene ethers, sulfoxides, sulfones, as well as polyethyleneglycol units (n=1-3) are well tolerated in the pleconaril derivative E shown below:

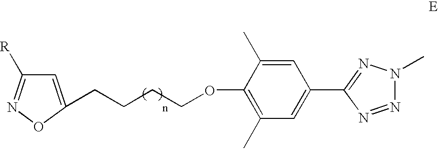

where n is from 1 to 3.

Thus, the R-group shown in compound E is clearly a suitable attachment point for a linker. Other examples of suitable compounds are those which can be covalently attached to derivatives of compound F through a linker, in a manner previously described, with a heterocyclic ring such as, but not limited to, an isoxazole ring, in a manner other than via the R group.

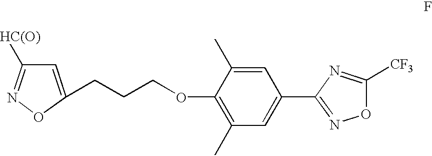

Multibinding antiviral compounds show an increase in potency as previously described and initially are assayed for antiviral activity as follows:

HeLa cells are plated in monolayers in 96 well plates and infected with a dilution of virus such that a 80-100% cytopathic effect is observed in 3 days. Compounds are serially diluted into wells and the cells are incubated for 3 days. Gluteraldehyde and crystal violet are added to the wells, the wells rinsed, dried and the amount of stain remaining in the wells is measured as an indication of the amount of intact cells. Data is reported as the amount of compound required to maintain 50% (MIC₅₀) or 80% (MIC₈₀) of the cells protected from virus-induced cytopathic effect (CPE). A panel of the available serotypes for rhinovirus, enterovirus, and hepatovirus is used, however, and initial evaluation of activity against HRV-1A, HRV-14, and coxsackievirus A21 is sufficient.

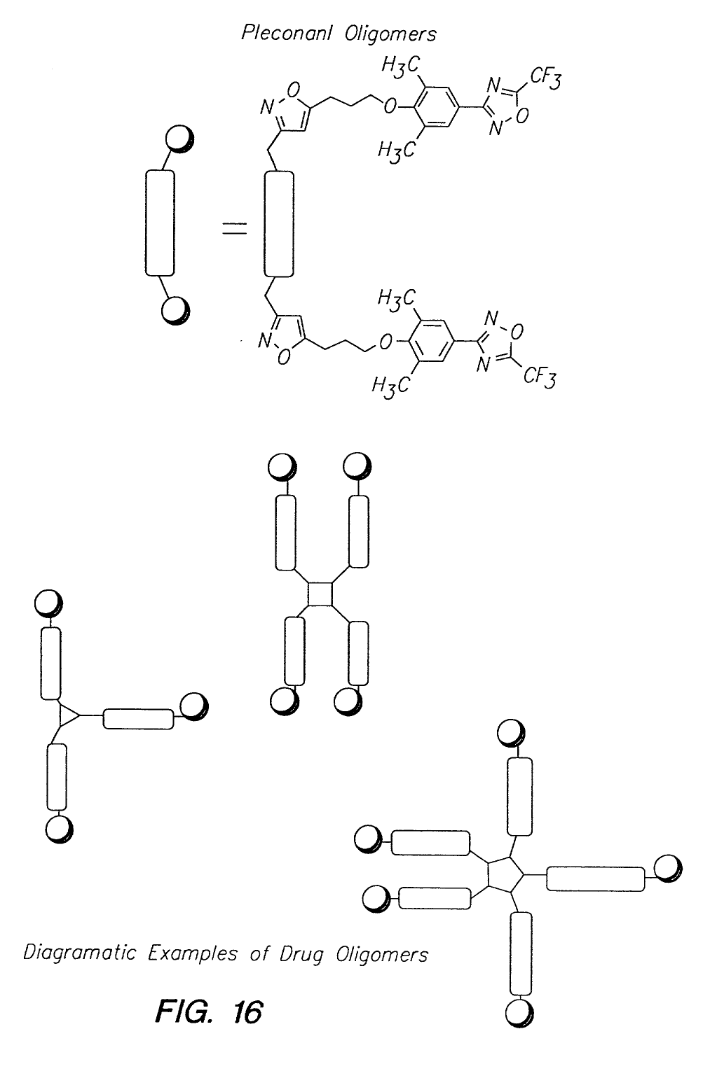

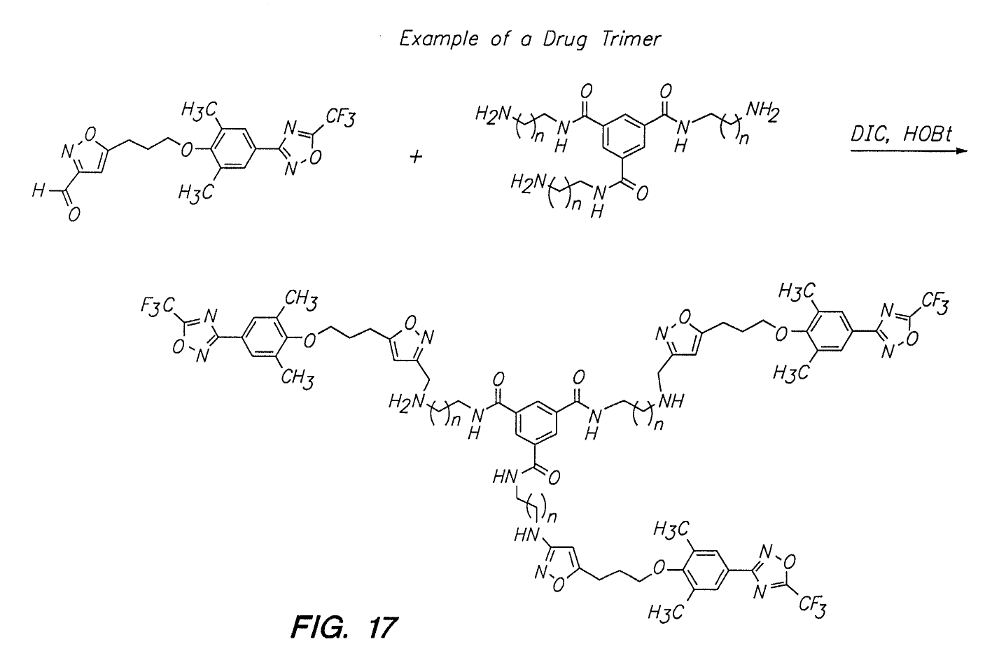

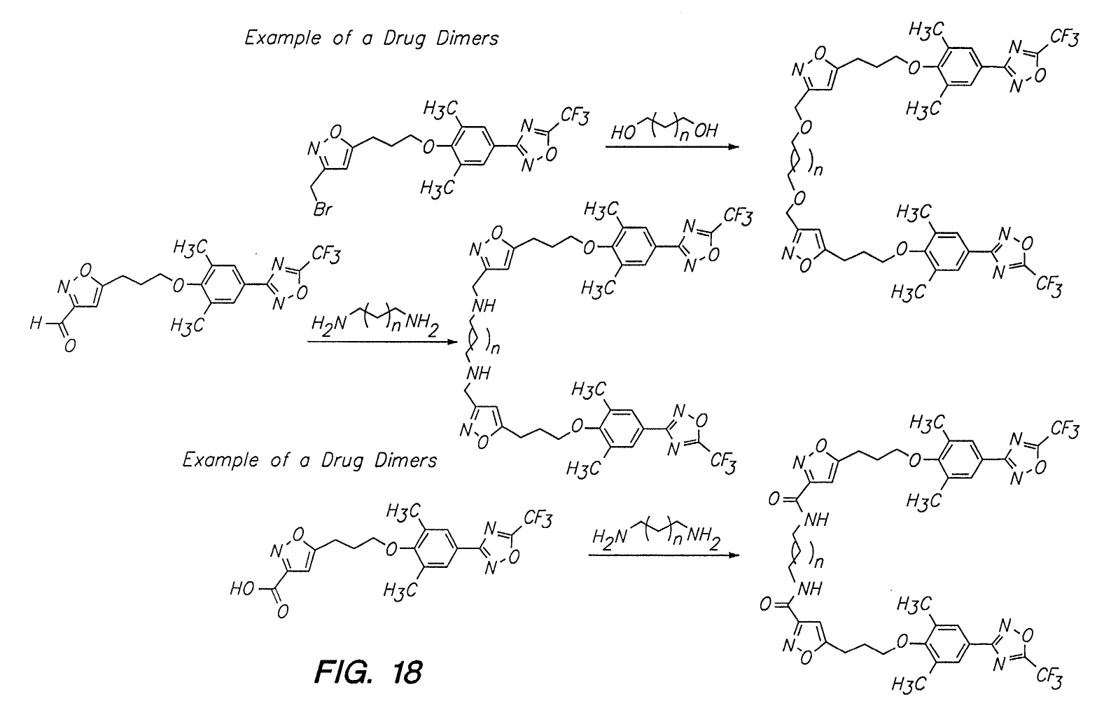

FIG. 16 illustrates examples of multibinding compounds based on comound F and FIGS. 17 and 18 illustrate examples for the synthesis of such compounds.

Amphotericin

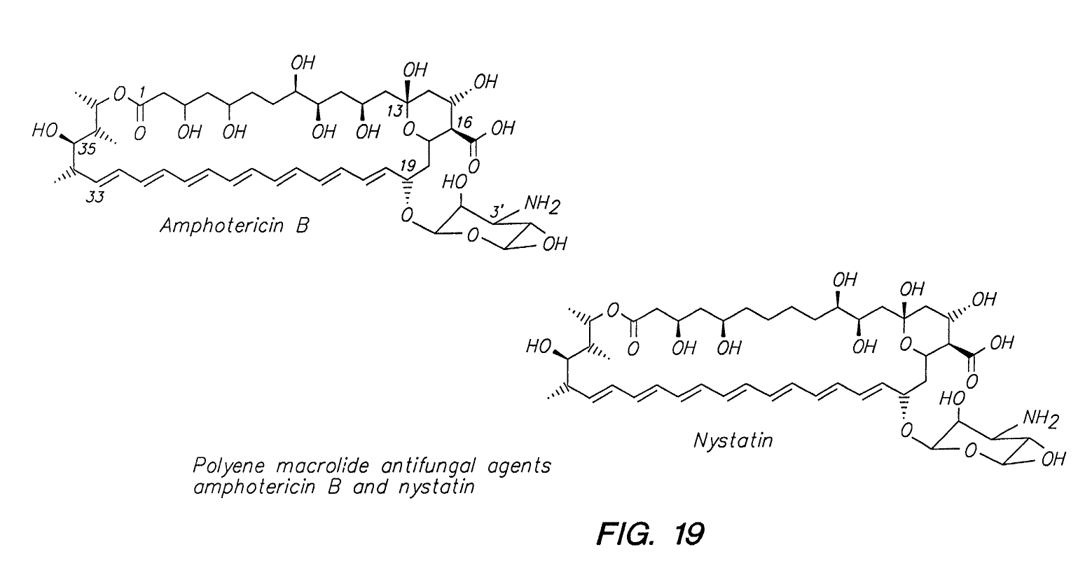

Amphotericin is an antibiotic that is active against fungal organisms and Leishmania parasites. Amphotericin B prototypifies the polyene macrolide family of antifungal agents, which also includes nystatin (FIG. 19) and the structurally simpler analogs filipin and roflamycoin.^{6, 7}

Amphotericin's target is a macromolecular structure, the fungal/parasite cell membrane. More specifically, amphotericin forms a complex with the sterol ergosterol within the cell membrane. This event increases the permeability of the fungal cell membrane, leading to loss of essential intracellular components. At low concentrations of amphotericin, loss of cell contents is restricted to cations and protons; however, higher concentrations or prolonged exposure results in loss of other cellular components, metabolic disruption, and cell death.⁸ Amphotericin will also complex with cholesterol within the context of mammalian cell membranes; however, this interaction is of lower affinity than is the interaction with ergosterol, leading to selectivity for fungal cells.^{9, 10}

In the most widely accepted model, 8-10 molecules of amphotericin assemble into a cyclic array with intercalated ergosterol molecules within a single leaflet of the fungal cell membrane.^{11, 12} This amphipathic “barrel stave” structure encloses an aqueous pore. Alignment of two such pores, one in each of the outer and inner membrane leaflets, produces a transmembrane channel that allows for ion and small non-electrolyte passage. Alternatively, single pores may sufficiently disrupt the adjacent membrane leaflet so as to produce leakage. The pores are believed to have an inner diameter of 8 Å, based on the finding that membranes bearing nystatin:sterol pores do not pass molecules larger than glucose.^{11, 13}

Amphotericin B has been in clinical use for more than 30 years and remains the “gold standard” for systemic treatment of serious, deep-seated mycoses due to its broad spectrum of action, its fungicidal properties, and the fact that resistance has been slow to develop and spread. With regard to amphotericin resistance, this typically involves alterations in fungal sterol biosynthesis leading to reduction or elimination of ergosterol production. Amphotericin B represents front-line therapy for invasive aspergillosis, blastomycosis, coccidioidmycosis, cryptococcosis, histoplasmosis, mucormycosis, and sporotrichosis. Likewise, nystatin is commonly used for treatment of superficial candidiasis.¹⁴ The primary drawbacks to amphotericin use are wide variety of side effects observed upon its administration. These side effects include fever, chills, anorexia, nausea, vomiting, headache, hypotension, flushing, vestibular disturbances, hyperkalemia, arrythmias, apnea, convulsions, and anaphylaxis observed during or shortly after infusion, as well as thrombophlebitis, nephrotoxicity, and hematologic disorders (anemia, thrombocytopenia, eosinophilia, agranulocytosis). Among these side effects, nephrotoxicity is the most common and the most important; indeed, nephrotoxicity is the side effect which most often limits clinical use of amphotericin. The nephrotoxic effects observed include both tubular defects (renal tubular acidosis, renal concentrating defect, hypokalemia and renal potassium losing, other electrolyte disorders) and decreased glomerular filtration rate. Recent studies suggest that amphotericin's ability to alter cell membrane permeability also leads to alterations in tubular and smooth muscle cell function, leading to tubular transport defects and vasoconstriction.¹⁵ Thus, approaches that decrease the affinity of amphotericin for mammalian cell membranes and/or decrease the relative delivery of amphotericin to mammalian versus fungal cell membranes may reduce the nephrotoxicity of the drug. It has been shown that various liposomal and lipid formulations of amphotericin decrease toxic side effects, presumably by altering the drug's delivery parameters.^{16, 17} Yamashita, Regen and other co-workers have prepared and studied conjugates of amphotericin B with a series of oligo(ethyleneglycol) appendages.¹⁸ The appendages were attached to the carboxyl group of the antibiotic through formation of an amide bond. Experiments with these compounds revealed that oligo(ethyleneglycol) conjugation significantly increased the solubility of amphotericin and decreased its aggregation and hemolytic properties while having only a modest negative impact on its in vitro potency. These results suggest that amphotericin's beneficial therapeutic effects may be separated from its toxic effects. However, it is to be noted that neither lipid formulation nor oligo(ethyleneglycol) conjugation increases the intrinsic antifungal potency of amphotericin B.

The amphotericin-associated membrane pore structures are believed to consist of 8-10 ergosterol-templated polyene macrolide subunits. Given this, linkage of 2 or more subunits with an appropriate framework (linker) increases the propensity to form the pore structure by pre-organizing the subunits for interaction with the membrane sterol. Amphotericin multibinding compounds also display decreased off-rates from fungal cell membranes. These phenomena translate into increased potency relative to amphotericin B as well as increased duration of action and post-antibiotic effect. It is also possible to link amphotericin subunits in a manner which serves to align pores in the inner and outer membrane leaflets, leading to increased efficiency of transmembrane channel formation and elution of cell contents which translates into increased potency and kill-rates. Pre-organization of the amphotericin subunits also enhances the specificity of association with ergosterol versus cholesterol, thereby providing a basis for decreasing the relative propensity for pore-based disruption of mammalian versus fungal cell membranes. Use of frameworks which increase the solubility and critical micelle concentrations of amphotericin multibinding compounds relative to amphotericin B serves to decrease non-specific membrane-disruptive effects. Finally, multivalency-based enhancement in affinity for ergosterol and structural analogs displayed by amphotericin multibinding compounds allows them to exert antifungal effects even against amphotericin-resistant organisms, which typically decrease or eliminate the production of ergosterol.¹⁹ However, it is to be noted that alteration of lipid composition may in some cases be insufficient to account for polyene resistance.²⁰

A typical yeast cell has a surface area of 40 square micrometers. Ergosterol and related sterols constitute approximately one-third (by weight) of all lipids present in yeast, a value similar to the sterol (mainly cholesterol) content of mammalian cell membranes. Thus, ergosterol is a major component of fungal cell membranes.

Amphotericin B is produced by the soil bacterium Streptomyces nodusus.²¹ and a number of derivatives modified at several positions around amphotericin B have been prepared. The key observations to be made based on studies of these compounds include:

Opening of the macrocyclic ring, masking of the polyhydroxy moiety, removal of the polyene unit, and opening of the tetrahydropyranyl ring all substantially reduce or abrogate antifungal activity.²²⁻²⁶

Chemical modification of the C16 carboxyl group as esters, amides, and a hydrazide often affords compounds with decreased hemolytic activity towards mammalian erythrocytes in vitro.^{18, 27-29} Amphotericin B methyl ester and N-ornithyl amphotericin B methyl ester were found to be less nephrotoxic than amphotericin B in animal models and, for amphotericin B methyl ester, in clinical trials.^{30, 31} However, it appears that neurotoxicity halted further clinical development of these compounds.

Acylation of amphotericin's mycosamino group typically decreases activity, but aminoacylation or alkylation are typically less disruptive.^{32, 33} One method for N-alkylation involves conjugate addition to N-ethylmaleimide derivatives; indeed Czerwinski et al. report a monovalent adduct of amphotericin B with N,N′-1,6-hexandiyl-bis(maleimide).³² This compound is substantially less potent, exhibiting MIC values against yeasts that are more than 20-fold higher than those observed with amphotericin B. As with C16 carboxyl modifications, N-alkylation or N-aminoacylation often substantially increases solubility (to tens of milligrams/mL) relative to amphotericin B and increases the differences in concentrations at which the compounds kill fungal cells versus cause hemolysis.

The choice of carboxyl and amino protecting groups (allyl ester and N-Fmoc) allows the preparation of several derivatives of amphotericin B modified at the C-13 hemiketal position.³⁴ Whereas amphotericin B bears a hydroxyl group at C13, the ketal derivatives made bear methoxy, beta-hydroxyethyoxy, and n-propyloxy substituents. In vitro experiments indicate that the ketal derivatives are substantially less hemolytic than is amphotericin B (with EH5O values increased at least 20-fold). The methoxy and beta-hydroxyethoxy ketals exhibited MIC values against a range of fungal pathogens that were on average 2-fold higher than those for amphotericin B. The n-propyloxy ketal was on average 10-fold less potent in vitro.

In summary, amphotericin B can be modified at a selected subset of functional groups. These modifications often reduce the hemolytic potential of the products, which are potentially useful in reducing the toxicity of the drug. However, in no case has semisynthetic modification of amphotericin B produced a derivative with anything more than a modest increase in in vitro potency. It would seem that an approach that affords derivatives with both enhanced potency and solubility would represent a significant advance.

In the “barrel stave” model for amphotericin B, 8-10 polyene macrolide subunits comprise a cyclic array with the polar mycosamine “tops” located at the membrane/aqueous pores and the C13-C17 hemiketal/C 16 carboxyl/C19-interface either inside or outside the cell. The C34-C37 “bottoms” are located in the middle of the plasma membrane. The polyhydroxlated C1-C12 edges are directed to the inside of the pores, while the heptaenic C20-C33 edges are directed towards the surrounding membrane. The amphotericin B subunits interleave around the perimeter of the pores; i.e., in a head-to-tail fashion. If the inner diameter of the pores is on the order of 8 Å,^{11, 13} the maximum distance between subunits across the pores is probably no more than 20 Å. This distance and geometry information, along with what is known about the effects of semisynthetic modifications of amphotericin B, suggests several approaches to the design of multibinding compounds that will pre-organize amphotericin B (or other polyene macrolide) subunits for formation of individual pores.

One linkage strategy involves attaching the subunits through their bottoms. In the case of amphotericin B, this requires selective functionalization of the C35 hydroxyl group.

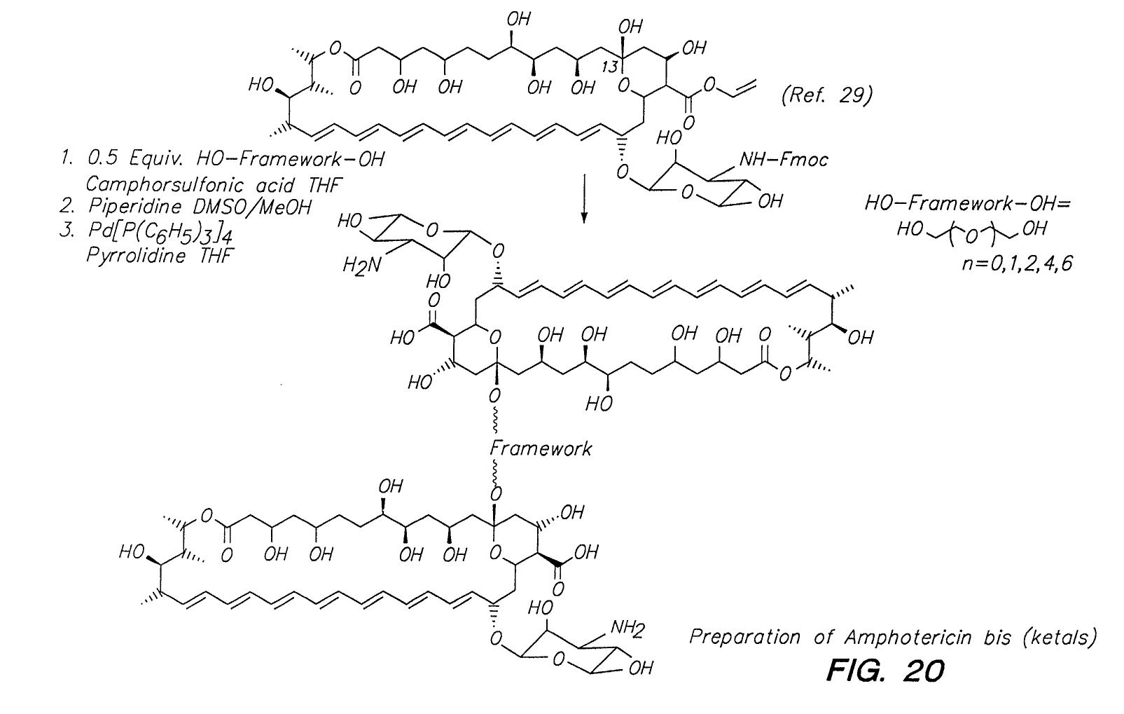

Additional and more straightforward linkage strategies involve attaching subunits through groups on the hydrophilic top of the molecule: The C13 hemiketal site, the C16 carboxyl, and the C3′ amino position of the mycosamine linked as an alpha-pyranoside to the C19 position of amphotericin B. Among these attachment sites, the C16 carboxyl and the mycosamine amino group are most preferred in light of the better understood medicinal chemistry invoking these positions and the fact that multiple substitutions of these positions are known that do not substantially decrease activity. The medicinal chemistry work carried out at C13 is much more limited and the n-propyloxy ketal is markedly reduced in in vitro activity, indicating that attachment to more extensive frameworks through this position is not preferred. The model for the pore also indicates that groups attached to the C13 position may lie within the pore itself which could potentially plug the pore. As illustrated in FIG. 20, it is straightforward to synthesize a series of oligo(ethyleneglycol) linked bis-ketals wherein framework refers to the linker.

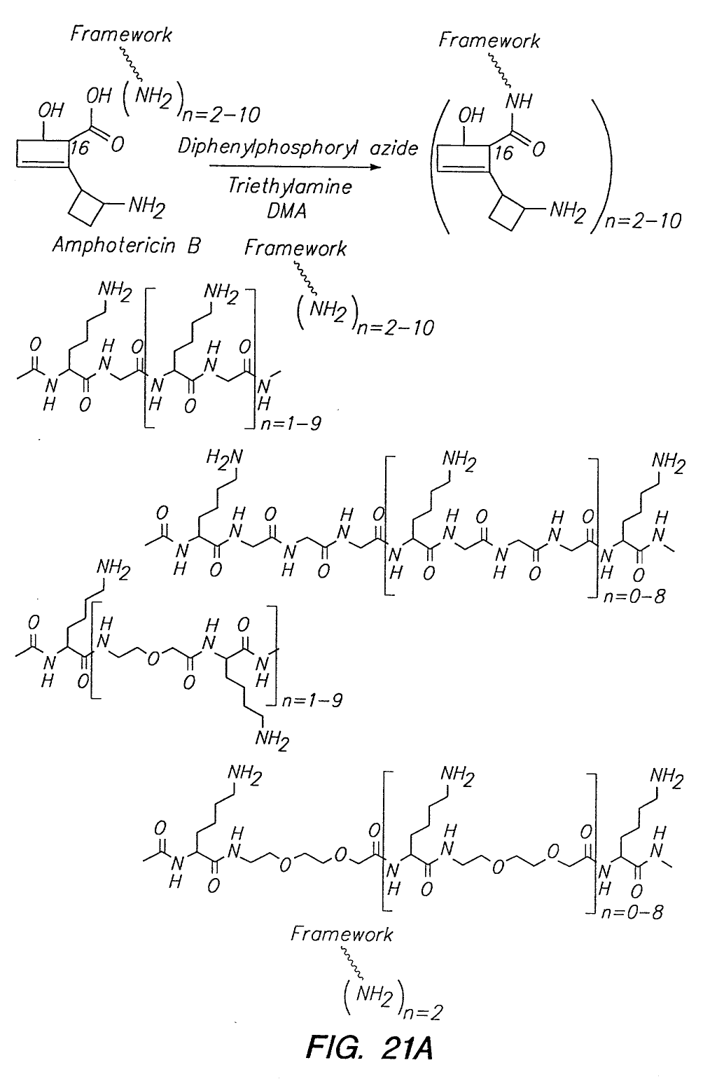

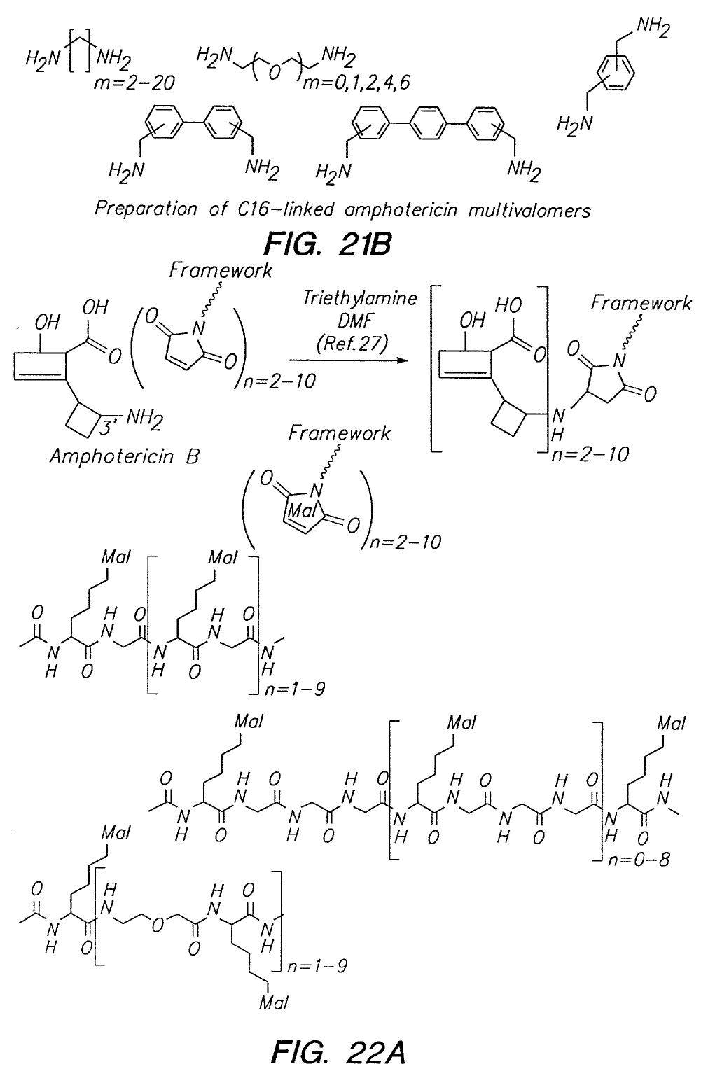

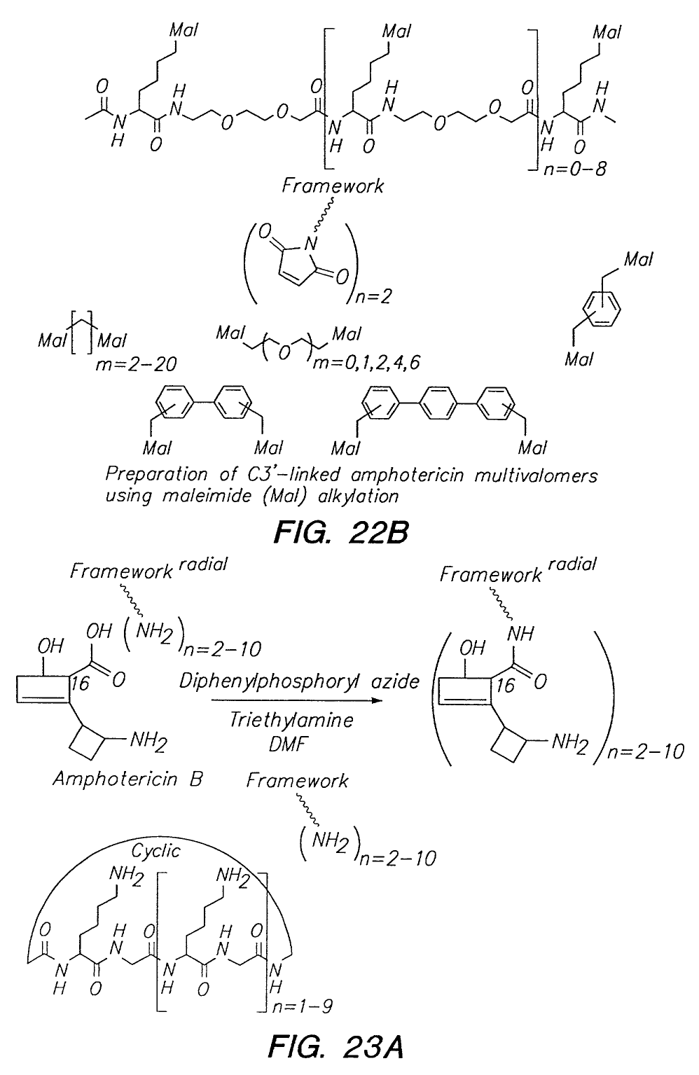

One preferred embodiment for amphotericin multibinding compounds would be to connect 2-10 of such ligands to a somewhat flexible linear framework such as an oligo(peptide) or a substituted oligo(ethyleneglycol). The attachment points for the subunits could be via the C16 carboxyl, the C3′ amino group, or a mixture of the two. Most preferred are dimers (divalomers) attached in either head-to-head (i.e., C16-C16), tail-to-tail (C3′-C3′), or head-to-tail (C16-C3′) fashion. Using a value of 8 Å for the internal diameter of the pore, 16 Å for the external diameter of the pore, and 8-10 subunits per pore, it is estimated that the minimum and maximum distances between C16 carboxyl groups and C3′ amino groups of adjacent subunits are approximately 5 and 15 Å, respectively. These distances could be spanned by attaching amphotericin subunits to every second to every fourth residue in an oligopeptide derived from alpha-amino acids, or to every other to every fourth monomer subunit of an oligo(ethyleneglycol). Examples of such structures and how they are prepared are shown in FIGS. 21 and 22. The preferred attachment chemistries include amide bond formation between amphotericin carboxyl groups and framework amino groups, conjugate or reductive alkylation of C3′ amino groups with framework, and amide or amine bond formation between the amino group of aminoacylated amphotericin derivatives and framework carboxyl or maleimide groups, respectively. Couplings are preferably carried out in polar yet inert solvents such as DMF using standard phosphoryl, uronium ion, or carbodiimide peptide coupling reagents including DPPA, PyBOP, HBTU, and DCC. Multibinding compound products are preferably isolated in crude form by precipitation from the reaction mixture by dilution into a non-polar solvent such as diethyl ether. Crude products are preferably purified by recrystallization, normal or reverse-phase chromatography, size-exclusion chromatography, or ion-exchange chromatography.

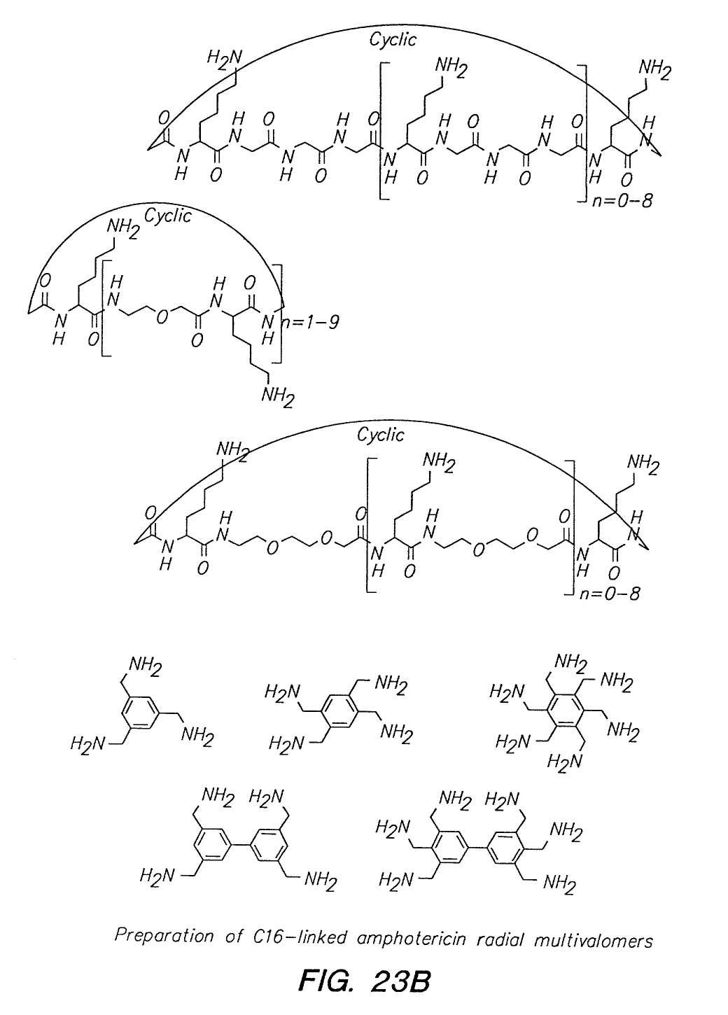

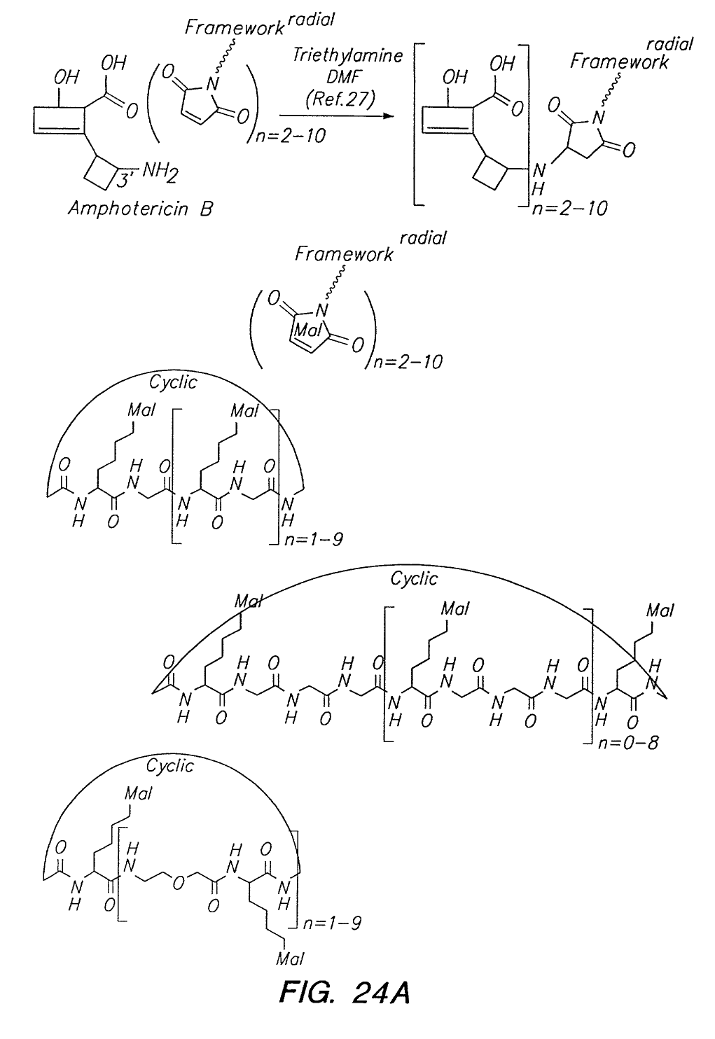

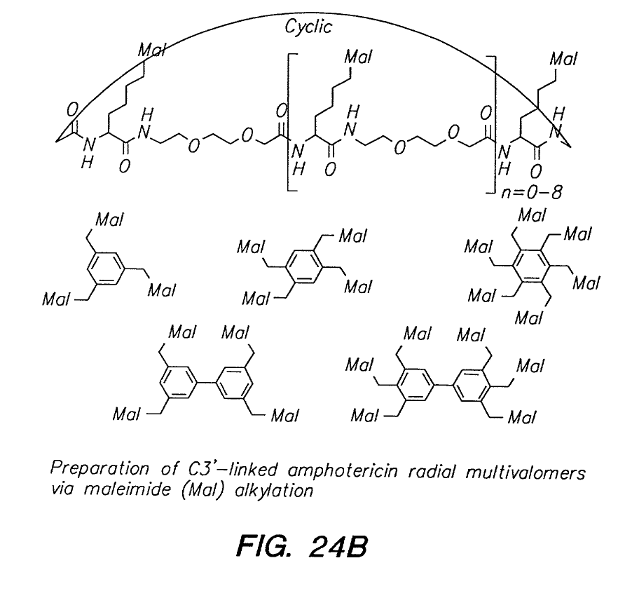

In a second preferred embodiment, 3-10 polyene macrolide subunits are radially displayed through attachment to a framework consisting of multiple “arms” that radiate from a central core structure such as a benzene ring. The arms of these structures must be sufficiently long to span the pore structure; i.e. they must allow for separations of individual subunits of up to approximately 20 Å. As in the previously described linear embodiment, amphotericin subunits are preferably attached to radial frameworks via C16 carboxyl groups and/or C3′ amino groups (FIGS. 23 and 24). The preferred attachment chemistries include amide bond formation between amphotericin carboxyl groups and framework amino groups, conjugate alkylation of C3′ amino groups with framework maleimide groups, and amide or amino bond formation between the amino group of aminoacylated amphotericin derivatives and framework carboxyl or maleimide groups, respectively. Couplings are preferably carried out in polar yet inert solvents such as DMF. Multibinding compound products are preferably isolated in crude form by precipitation from the reaction mixture by dilution into a non-polar solvent such as diethyl ether. Crude products are preferably purified by recrystallization, normal- or reverse-phase chromatography, size-exclusion chromatography, or ion-exchange chromatography. Additional important aspects of the design and use of radial amphotericin displays are as follows. First, it is to be noted that the central core of a radial framework is expected to sit over the pore and could potentially plug it up. Thus, it is important to allow for sufficient separation between the core and the subunits such that plugging does not occur. Second, it is not necessarily essential, and may in fact be preferable to attach all 8-10 subunits essential for formation of a single, complete pore to a single linker. In cases where the radially displayed multibinding compound is insufficient to form a completed pore, one would co-administer free amphotericin B or an alternate mono- or polyvalent polyene macrolide to fill in the “gaps”.

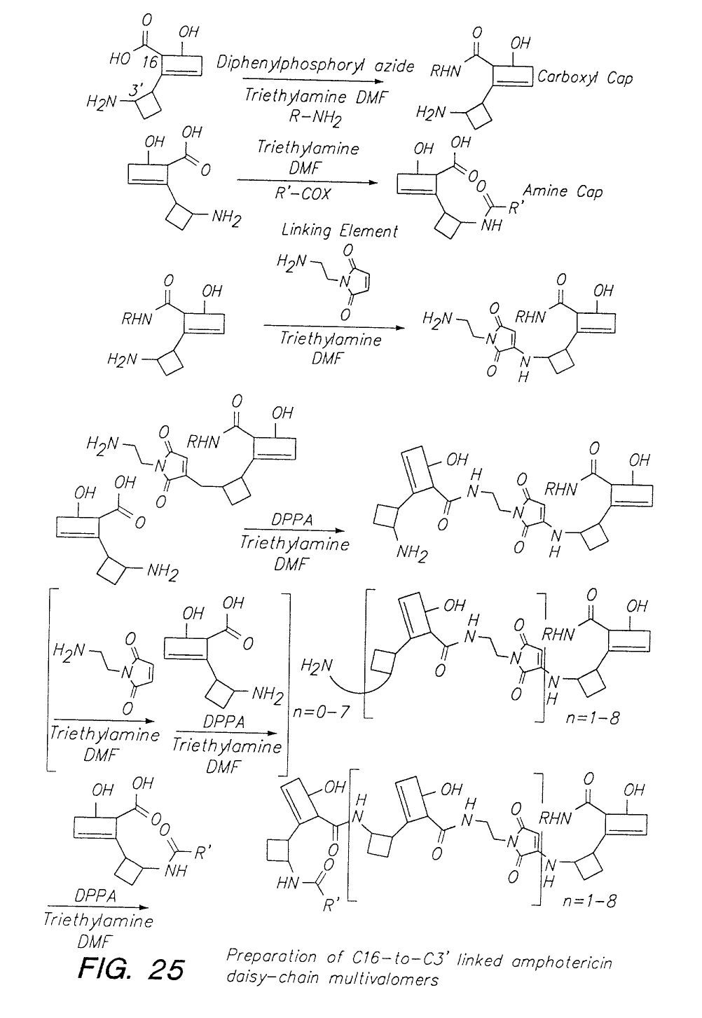

Given the presumed head-to-tail orientation of polyene macrolide subunits around the perimeter of the pore, a third preferred embodiment would be to connect 2-10 subunits in head-to-tail, daisy-chain fashion e.g., in a trimer (trivalomer) the C16 position of one terminal subunit could be attached to the C3′ position of the central subunit while the C16 position of the same central subunit could be attached to the C3′ position of the other terminal subunit. In such structures, the linker is discontinuous in that it is interrupted by the polyene macrolide subunits. In other words, portions of the macrolides themselves contribute to the overall linker (framework) structure. As noted above, the estimated distance between juxtaposed C16 carboxyl groups and C3′ amino groups is on the order of 5 Å; thus, relatively short linking elements are preferred. One such example is provided in FIG. 25. The preferred attachment chemistries include amide bond formation between amphotericin carboxyl groups and linker amino groups, conjugate or reductive alkylation of C3′ amino groups with linker maleimide groups, and amide or amine bond formation between the amino group of aminoacylated amphotericin derivatives and linker carboxyl or maleimide groups, respectively (here the aminoacyl addend can comprise part or all of the linker). Couplings are preferably carried out in polar yet inert solvents such as DMF. Multibinding compound products are preferably isolated in crude form by precipitation from the reaction mixture by dilution into a non-polar solvent such as diethyl ether. Crude products are preferably purified by recrystallization, normal- or reverse-phase chromatography, size-exclusion chromatography, or ion-exchange chromatography.

It is noted that the most preferred amphotericin multibinding compound may contain more than one distinct multivalent construct admixed with one or more monovalent constructs.

It is also to be noted that multivalent amphotericin B macromolecular ligands (constructs) could be designed such that different ligands reside in different pores. The linkers for such ligands would be fairly rigid and with distance and geometric constraints that would make it impossible for the attached ligands to be simultaneously incorporated into the same pore. Such multibinding compounds, by themselves or perhaps in admixture with amphotericin B or other monovalent or multivalent polyene macrolide derivatives, would serve to nucleate multiple pores in close proximity. It is envisioned that such an event would enhance leakage of fungal cell contents and killing activity. Two distinct types of adjacencies are possible, “side-by-side” in the same leaflet of the cell membrane, or “bottom-to-bottom” spanning both membrane leaflets. Bottom-to-bottom adjacency is especially attractive, as it would serve to nucleate channels that span the entire membrane. The barrier to realizing such constructs will be the development of efficient and selective chemistry for linking subunits through their bottom edges, for example the C35 hydroxyl group of amphotericin B. With regard to side-by-side pores, the optimal nucleating multibinding compounds are envisioned to be those with rigid, divergently arrayed frameworks which actually prevent the attached amphotericin B subunits from residing within the same pore. Plausible attachment points for the subunits of such structures include C13, C16, mycosamine, and C35. Given the divergent enforcement in these systems, it may be required that “proximal pore nucleating elements” be co-administered with amphotericin B or other monovalent or multivalent polyene macrolide derivatives.

Standard in vitro antifungal susceptibility studies involving broth dilution, plate dilution, or zone of inhibition assays are used as the first step in analyzing the properties of amphotericin multibinding compounds. These experiments provide minimum inhibitory concentrations (MICs) and minimum fungicidal concentrations (MFCs). Controls for these studies include amphotericin B, monovalent amphotericin B-framework conjugates, and fluconazole. The panel or organisms screened includes the following organisms in both drug-sensitive and drug-resistant strains:

Saccharomyces cerevisiae

Candida albicans

Non-albicans species of Candida

Aspergillus niger

Aspergillus nidulans

Aspergillus fumigatus

Aspergillus flavus

Cryptococcus neoformans

Coccidioides immitis

Torulopsis glabrata

Histoplasma capsulatum

Blastomcyes dermatitiddis

Compounds which display promising in vitro activity are advanced to further biological and pharmacological analysis. Important additional in vitro experiments include determinations of time-kill profiles, hemolytic activities, as well as toxicities toward cultured human cells and tissue samples. Important in vivo experiments include determination of pharmacokinetic parameters, efficacy in mouse models of infection, and analysis of acute tolerability and toxic effects, particularly nephrotoxicity and neurotoxicity.

The following examples are specific to Amphotericin and related polyene macrolide compounds.

Synthesis of bis(amphotericin) ketals (FIG. 20).

A solution of N-(9-fluorenylmethoxycarbonyl)amphotericin B allyl ester (2.16 mmol, 2.51 g) and ethylene glycol (1.1 mmol, 0.067 g) in 25 mL dry THF is cooled in an ice bath and treated with camphorsulfonic (0.58 mmol, 0.145 g). After stirring for 30 minutes, ethyl acetate and sodium bicarbonate solution are added. After standard work-up, the crude product is chromatographed on silica gel using 10:1 methylene chloride:methanol eluent to afford the di-Fmoc, di-allyl ester bis(ketal). The Fmoc groups of the bis(ketal) are then removed at room temperature using a two-fold excess of piperidine in DMSO:methanol solvent. The crude product is precipitated from the reaction mixture by pouring it into diethyl ether. After drying in vacuo, the allyl esters are removed at room temperature using a combination of tetrakis(triphenylphosphine)palladium[0] and pyrrolidine in THF solvent. The crude product precipitates from the reaction mixture, is collected by centrifugation, washed with THF, and then re-precipitated by dissolving in a 5:1 mixture of methanol:THF and diluting into diethyl ether. Drying in vacuo affords 13-0, 13′-0′-(1,2-ethandiyl)bis(amphotericin B).

Synthesis of C16-linked linear- and radial amphotericin B multibinding compounds (FIGS. 21 and 23).

Amphotericin B (0.0554 g, 0.060 mmol) is suspended in 2 mL of DMA, stirred at room temperature, and then treated with triethylamine (0.60 mmol), DPPA (0.60 mmol), and 1,11-diamino-3,6,9-trioxaundecane (0.0058 g, 0.030 mmol) in the dark under a nitrogen atmosphere. After 5 days, the reaction mixture is poured into 80 mL of diethyl ether. The crude product is further purified, first by four times resolubilizing in methanol and reprecipitating from diethyl ether, and then by silica gel chromatography using 9:1 methanol:30% ammonium hydroxide eluent to afford bis(amphotericin B)-3,6,9-trioxaundecan-1,1 1-diamide

Synthesis of C3′-linked linear- and radial amphotericin B multibinding compounds (FIGS. 22 and 24). To a stirred suspension of amphotericin B (0.277 g, 0.30 mmol) in 5 mL DMF is added triethylamine (0.042 mL, 0.300 mmol) and then N,N′-1,6-hexandiyl-bis(maleimide) (0.041 g, 0.15 mmol). The mixture is stirred at room temperature for 1 day. The crude product is precipitated by the addition of 250 mL diethyl ether, washed with additional ether, and dried in vacuo. This material is further purified by silica gel chromatography using 13:6:1 chloroform:methanol:water eluent to afford bis(amphotericin B)-N,N′-1,6-hexandiyl-bis(succinamide).

Synthesis of C16-to-C3′ linked amphotericin B daisy-chain multibinding compounds (FIG. 25). Amphotericin B (0.554 g, 0.60 mmol) is suspended in 20 mL of DMA, stirred at room temperature, and then treated with triethylamine (6.0 mmol), DPPA (6.0 mmol), and 1-aminopropane (0.034 g, 0.60 mmol) in the dark under a nitrogen atmosphere. After 5 days, the reaction mixture is poured into 800 mL of diethyl ether. The crude product is further purified, first by four times resolubilizing in methanol and reprecipitating from diethyl ether, and then by silica gel chromatography using 9:1 methanol:30% ammonium hydroxide eluent to afford the “carboxyl capped” adduct amphotericin B-1-propanamide.

To a stirred solution of amphotericin B-1-propanamide (0.579 g, 0.60 mmol) in 5 mL dimethylformamide is added triethylamine (0.084 mL, 0.600 mmol) and then N-(2-aminoethyl)maleimide (0.084 g, 0.060 mmol). The mixture is stirred at room temperature for 1 day. The crude product is precipitated by the addition of 250 mL diethyl ether, washed with additional ether, and dried in vacuo. This material is further purified by silica gel chromatography using 9:1 methanol:30% ammonium hydroxide eluent to afford amphotericin B-(1-propanamide)(N-(2-aminoethyl)succinimide).

Amphotericin B-(1-propanamide)(N-(2-aminoethyl)succinimide) (0.332 g, 0.30 mmol) is suspended in 10 mL of DMA, stirred at room temperature, and then treated with amphotericin B (0.277 g, 0.30 mmol) followed by triethylamine (3.0 mmol) and diphenylphosphoryl azide (DPPA, 3.0 mmol) in the dark under a nitrogen atmosphere. After 2 days, the reaction mixture is poured into 400 mL of diethyl ether. The crude product is further purified, first by four times resolubilizing in methanol and reprecipitating from diethyl ether, and then by silica gel chromatography using 9:1 methanol:30% ammonium hydroxide eluent to afford the carboxyl-capped head-to-tail dimer [amphotericin B-(1-propanamide)(N-(2-amidoethyl)succinimide)]-[amphotericin B].

[Amphotericin B-(1-propanamide)(N-(2-amidoethyl)succinimide)]-[amphotericin B] (0.201 g, 0.10 mmol) is dissolved in 2 mL of DMF and treated with triethylamine (0.014 mL, 0.10 mmol) followed by N-(2-amino-ethyl)maleimide (0.014 mL, 0.10 mmol). The mixture is stirred at room temperature for 1 day. The crude product is precipitated by the addition of 50 mL diethyl ether, washed with additional ether, and dried in vacuo. This material is further purified by silica gel chromatography using 9:1 methanol:30% ammonium hydroxide eluent to afford [amphotericin B-(1-propanamide)(N-2-amidoethyl)succinamide)]-[amphotericin B(N-(2-amidoethyl)succinamide)].

[Amphotericin B-(1-propanamide)(N-(2-amidoethyl)succinamide)]-[amphotericin B(N-(2-amidoethyl)succinamide)] (0.215 g, 0.10 mmol) is suspended in 5 mL of DMA stirred at room temperature, and then treated with N-acetyl-amphotericin B (0.097 g, 0.10 mmol) followed by triethylamine (1.0 mmol) and DPPA, 1.0 mmol) in the dark under a nitrogen atmosphere. After 2 days, the reaction mixture is poured into 100 mL of diethyl ether. The crude product is further purified, first by four times resolubilizing in methanol and reprecipitating from diethyl ether, and then by silica gel chromatography using 9:1 methanol:30% ammonium hydroxide eluent to afford the carboxyl- and amide-capped head-to-tail/head-to-tail trimer [amphotericin B(1-propanamide)(N-(2-amidoethyl)succinamide)]-[ampphotericin B(N-(2-amidoethyl)succinamide)]-[N-acetyl-amphotericin B].

Multibinding Modifiers of Cellular Filamentous Structures

Drugs that modify the cellular filamentous structures are most commonly antineoplastic agents.³⁵

The repeating protein structures that make up the cyctoskeleton play a crucial role in a number of cellular processes. Members of this class of proteins include tubulin, actin, myosin, tropomyosin, troponin, titin, nebulin, α-actinin, myomesin, keratin, C-protein, dynein, and nexin. The repeating nature of these cytoskeletal structures make an approach to the production of modifiers based on polyvalency attractive. This discussion focuses, for illustrative purposes only, on the modifiers of tubulin polymerization and, to a lesser extent, actin. It is understood that the general principles will be applicable to the modification of other cellular filamentous structures.

Tubulin:

In vivo cellular assembly of α-tubulin and β-tubulin results in the formation of microtubules. Microtubule arrays in cells are labile. This lability is key to their cellular function and thus altering their stability can impact cellular processes. In particular, formation of a mitotic spindle is accompanied by alteration in the dynamic properties of microtubules. Thus, agents that influence the dynamic equilibrium between tubulin and microtubules can disrupt mitosis resulting in the death of dividing cells. This antimitotic activity of spindle poisons has been widely used for cancer chemotherapy.

There are two general classes of tubulin modulators, tubulin polymerization inhibitors and microtubule stabilizers.

Microtubule Stabilizers:

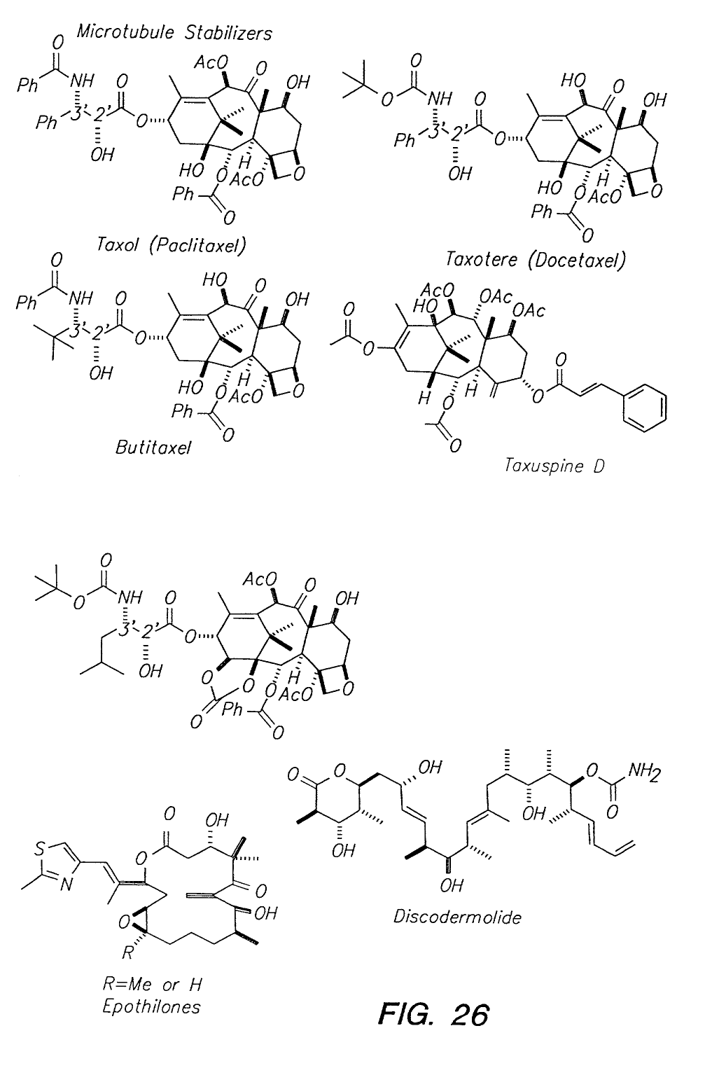

Numerous microtubule stabilizers are illustrated in FIG. 26.

Taxol or Paclitaxel is an antimicrotubule agent that promotes the assembly of microtubules from tubulin dimers and stabilizes microtubules by preventing depolymerization. This stability results in the inhibition of the normal dynamic reorganization of the microtubule network that is essential for vital interphase and mitotic cellular functions. In addition, paclitaxel induces abnormal arrays or “bundles” of microtubules throughout the cell cycle and multiple asters of microtubules during mitosis.

Taxotere or Docetaxel is an antineoplastic agent that acts by disrupting the microtubular network in cells that is essential for mitotic and interphase cellular functions. Docetaxel binds to free tubulin and promotes the assembly of tubulin into stable microtubules while simultaneously inhibiting their disassembly. This leads to the production of microtubule bundles without normal function and to the stabilization of microtubules, which results in the inhibition of mitosis in cells. Docetaxel's binding to microtubules does not alter the number of protofilaments in the bound microtubules, a feature which differs from most spindle poisons currently in clinical use.

For both Taxotere and Taxol, common side effects include neutropenia, leukopenia, and peripheral neuropathy. These side effects occur in a dose dependent manner. Other side effects include a hypersensitivity reaction.³⁶

Taxol and Taxotere are also subject to Multi Drug Resistance (MDR).³⁷ This limits the utility of the drug for repeated administrations. SAR analysis for taxol is provided in FIG. 27.

Microtubule Disrupters:

Microtubule disrupters include the following compounds, some of which are further elaborated upon:

1. Podophyllotoxin is a cytotoxic agent that has been used topically in the treatment of genital warts. It arrests mitosis in metaphase, an effect it shares with other cytotoxic agents such as the vinca alkaloids.³⁸ Podophyllotoxin is subject to Multi Drug Resistance (MDR)³⁷.

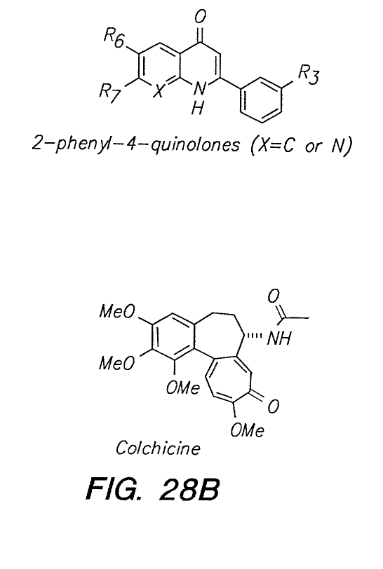

2. Colchicine.³⁹ which is illustrated in FIG. 28.

3. 2-phenyl-4-quinolones^{40, 41} which are illustrated in FIG. 28.

4. Combrestatins⁴² which are illustrated in FIG. 28.

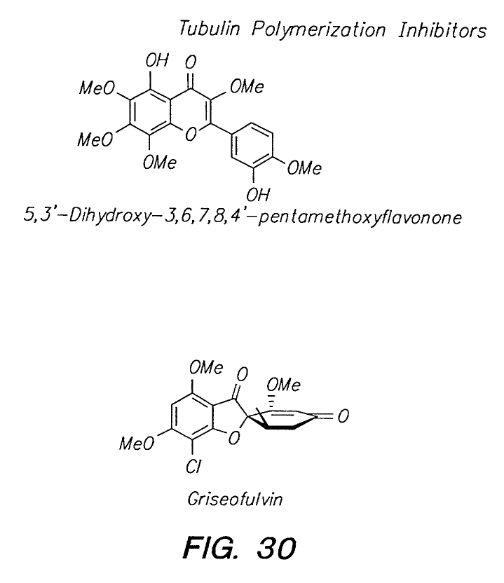

5. 5,3′-Dihydroxy-3,6,7,8,4′-pentamethoxyflavonone⁴³ which is illustrated in FIG. 30.

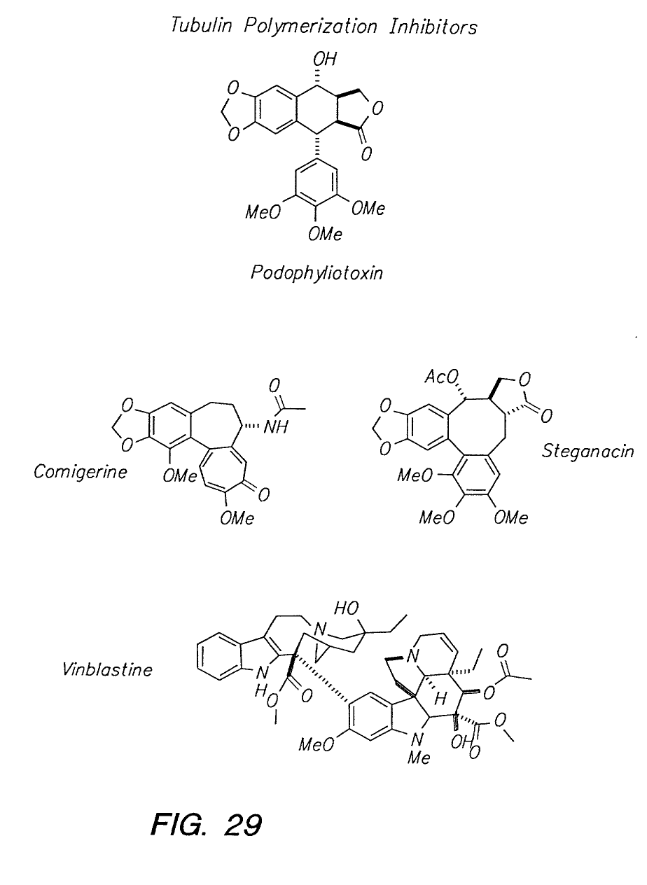

6. Coringerine and Steganacin.^{44, 45} which are illustrated in FIG. 29.

7. Noscapine.⁴⁶

Griseofulvin:

Griseofulvin, illustrated in FIG. 30, is fungistatic with in vitro activity against various species of Microsporum, Epidermophyton and Trichophyton. It has no effect on bacteria or other genera of fungi. Human pharmacology, following oral administration, griseofulvin is deposited in the keratin precursor cells and has a greater affinity for diseased tissue. The drug is tightly bound to the new keratin which becomes highly resistant to fungal invasions. However, griseofulvin is carcinogenic in laboratory animals.^{47, 48, 49, 50}

Vinca Alkaloids:

Experimental data indicate that the action of Velban (Vinblastine Sulfate—illustrated in FIG. 29—not as the sulfate salt) is different from that of other recognized antineoplastic agents. Tissue-culture studies suggest an interference with metabolic pathways of amino acids leading from glutamic acid to the citric acid cycle and to urea. In vivo experiments tend to confirm the in vitro results. A number of studies in vitro and in vivo experiments have demonstrated that Velban produces a stathmokinetic effect and various atypical mitotic figures. The therapeutic responses, however, are not fully explained by the cytologic changes, since these changes are sometimes observed clinically and experimentally in the absence of any oncolytic effects.

Reversal of the antitumor effect of Velban by glutamic acid or tryptophan has been observed. In addition, glutamic acid and aspartic acid have protected mice from lethal doses of Velban. Aspartic acid was relatively ineffective in reversing the antitumor effect. Other studies indicate that Velban has an effect on cell-energy production required for mitosis and interferes with nucleic acid synthesis. The mechanism of action of Velban has been related to the inhibition of microtubule formation in the mitotic spindle, resulting in an arrest of dividing cells at the metaphase stage.

Vincristine:

The mechanisms of action of Oncovin remain under investigation. The mechanism of action of Oncovin has been related to the inhibition of microtubule formation in the mitotic spindle, resulting in an arrest of dividing cells at the metaphase stage.

Cancer Treat. Rep. 60:127 (1976). Prepn and pharmacology of [3H]vincristine: Owellen, Donigian, J. Med. Chem., 15:894 (1972). Symposium on vincristine: Cancer Chemother. Rep. 52:453-535 (1968). Pharmacology: R. H. Adamson et al., Arch. Int. Pharmacodyn. Ther., 157:299 (1965); S. M. Sieber et al., Cancer Treat. Rep., 60:127 (1976). Symposium on vincristine: Cancer Chemother. Rep., 52:543-535 (1968). Comprehensive description of the sulfate: J. H. Burns, Anal. Prof. Drug Subs., 1:463-480 (1972).

Vinorelbine:

3′,4′-Didehydro-4′-deoxy-C′-norvincaleukoblastine, P. Mangeney, et al., Tetrahedron, 35:2175 (1979). Pharmacology: G. Math, et al., Cancer Letters, 27:285 (1985). Clinical pharmacokinetics: R. Rahmani, et al., Cancer Res., 47:5796 (1987). Symposium: Seminars in Oncology 16, Suppl 4:1-45 (1989). Review: M. Marty, et al., Nouv. Rev. Fr. Hematol, 31:77-84 (1989).

Vinorelbine is a vinca alkaloid that interferes with microtubule assembly. The vinca alkaloids are structurally similar compounds comprised of two multiringed units, vindoline and catharanthine. Unlike other vinca alkaloids, the catharanthine unit is the site of structural modification for vinorelbine. The antitumor activity of vinorelbine is thought to be due primarily to inhibition of mitosis at metaphase through its interaction with tubulin. Like other vinca alkaloids, vinorelbine may also interfere with: 1) amino acid, cyclic AMP, and glutathione metabolism, 2) calmodulin-dependent Ca⁺⁺-transport ATPase activity, 3) cellular respiration, and 4) nucleic acid and lipid biosynthesis. In intact tectal plates from mouse embryos, vinorelbine, vincristine, and vinblastine inhibited mitotic microtubule formation at the same concentration (2 mcM), inducing a blockade of cells at metaphase. Vincristine produced depolymerization of axonal microtubules at 5 mcM, but vinblastine and vinorelbine did not have this effect until concentrations of 30 mcM and 40 mcM, respectively. These data suggest relative selectivity of vinorelbine for mitotic microtubules.

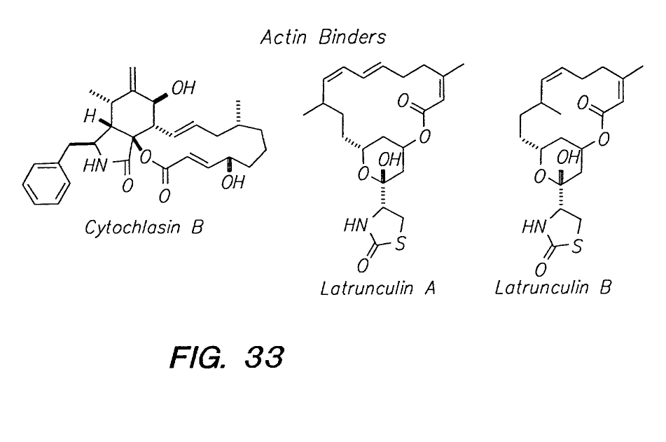

Actin Formation Blockers (as illustrated in FIG. 33):

Cytochalasins (more than twenty cytochalasins):

Major biological effects are the blockage of cytoplasmic cleavage by blocking formation of contractile microfilament structures, resulting in multinucleate cell formation, the reversible inhibition of cell movement, and the induction of nuclear extrusion: Carter, Nature, 213:261 (1967); Krishan, J., Cell Biol., 54:657 (1972); E. D. Korn, Physiol. Rev., 62:703 (1982). Correlation between effects of cytochalasins on cellular structures and cellular events and those on actin in vitro: I. Yahara, et al., J. Cell Biol., 92:69 (1982). Other reported effects include the inhibition of glucose transport, of thyroid secretion, of growth hormone release, of phagocytosis, and of platelet aggregation and clot contraction. See D. A. Hume, et al., Nature, 272:359 (1978). Nomenclature: M. Binder, et al., J. Chem. Soc., Perkin Trans. I, 1146 (1973). Reviews: M. Binder, et al., Chem. Int. Ed., 12:370 (1973); R. B. Herbert in “The Alkaloids”, vol. 7, J. E. Saxton, Ed. (The Chemical Society, London, 1977) pp. 29-30; W. G. Thilly, et al., Front. Biol., 46:53-64 (1978); L. V. Dommina, et al., Proc. Nat. Acad. Sci. USA, 79:7754-7757 (1982); W. Siess, et al., ibid., 7709-7713.

Latrunculins

Comparative study with cytochalsin D, q.v., on the effects on morphology, actin organization and cell processes: I. Spector, et al., Cell Motil. Cytoskel., 13:127 (1989).

Phalloidin

Toxin that acts by binding actin, q.v., an essential internal structural protein. Ultrastructural pathology: M. A. Russo, et al., Am. J. Pathol., 109:133 (1982). Toxicity study: Vogt, Arch. Exp. Pathol. Pharmakol., 190:406 (1938). Review of the chemistry and toxicology of the toxins of Amanita phalloides: Wieland, Wieland, Pharmacol. Rev., 11:87-107 (1959); see also T. Wieland, Fortschr. Chem. Org. Naturst., 25:214-250 (1967); T. Wieland, et al., Crit. Rev. Biochem., 5:185-260 (1978).

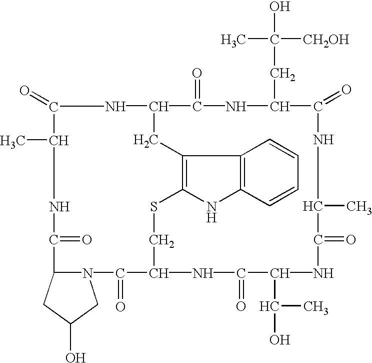

Amanitin

Review of chemistry and toxicology of the toxins of Amanita phalloides: Wieland, Wieland, Pharmacol. Rev., 11:87-107 (1959); T. Wieland, Fortschr. Chem. Org. Naturst., 25:214-250 (1967); T. Wieland, et al., Crit. Rev. Biochem., 5:185-260 (1978). Book: H. Faulstich, et al., “Amanita Toxins and Poisoning: International Amanita Symposium (Lubrecht Cramer, Heidelberg, 1980 246 pp.

Potential role(s) for multibinding compounds based on modifiers of cellular filamentous structures:

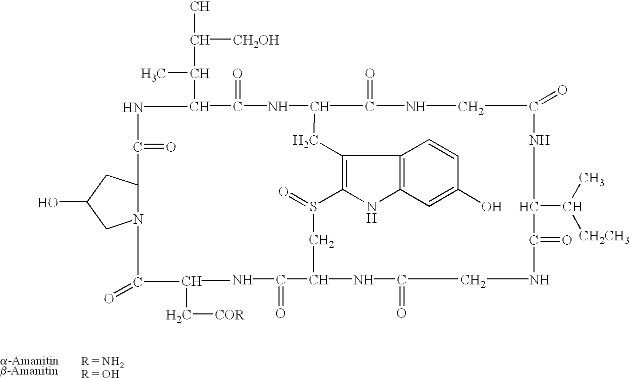

It is contemplated that multibinding compounds based on modifiers of cellular filamentous structures will increase potency of action at the desired targets while minimizing non-mechanism related side effects. Multibinding compounds also offer the possibility of minimizing efflux from resistant cells by maximizing the binding to the target (for example, tubulin).

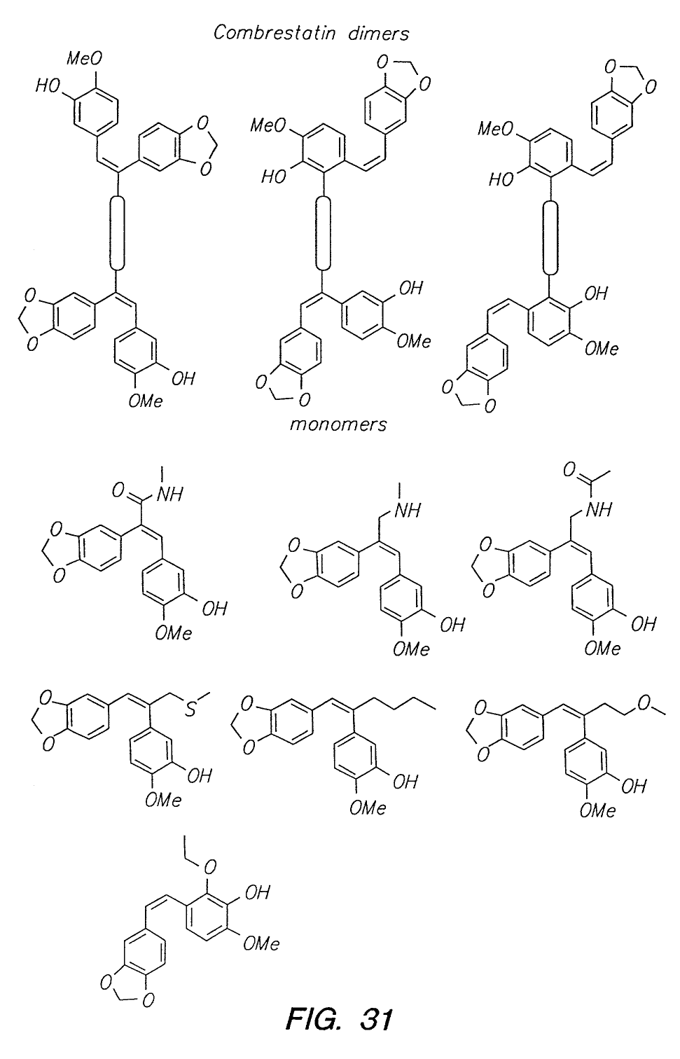

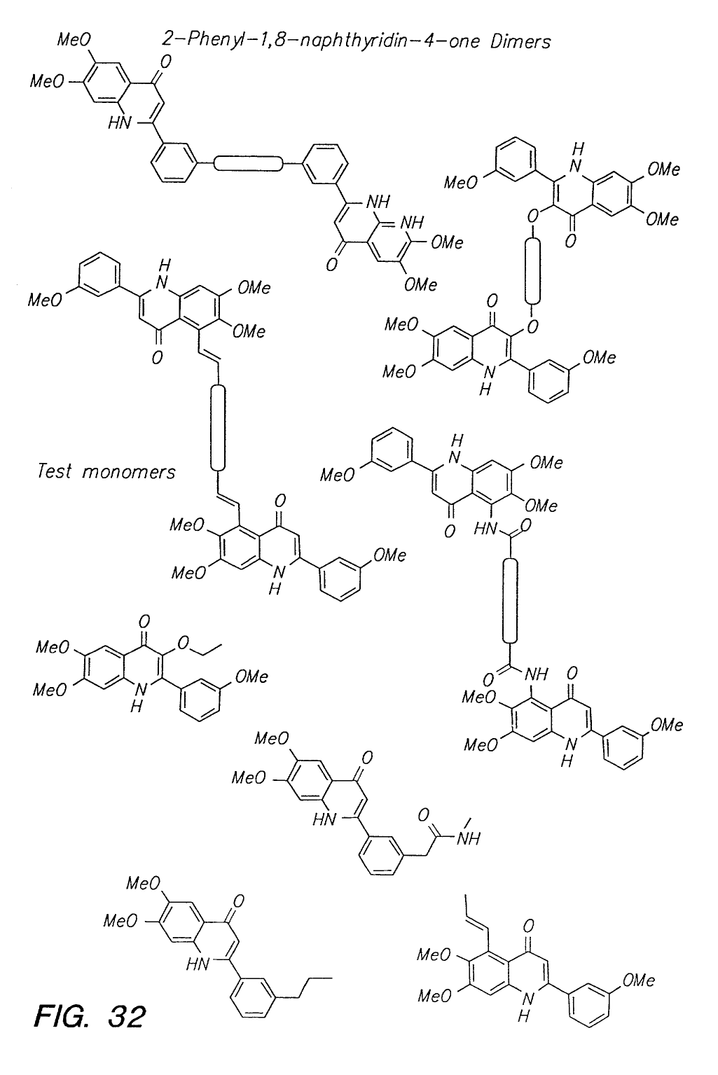

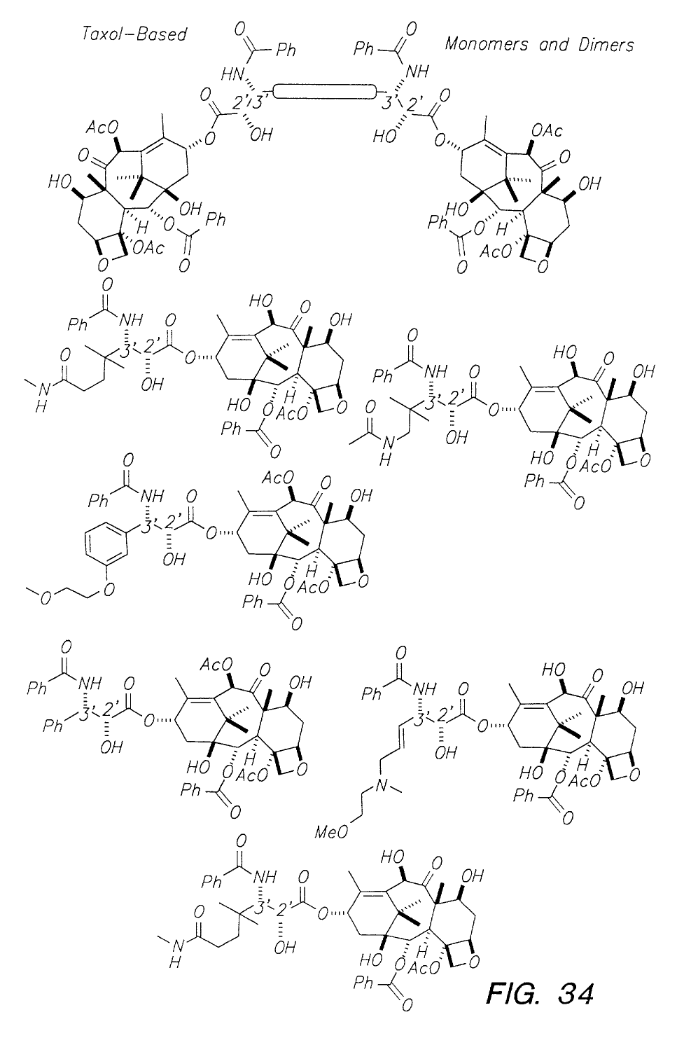

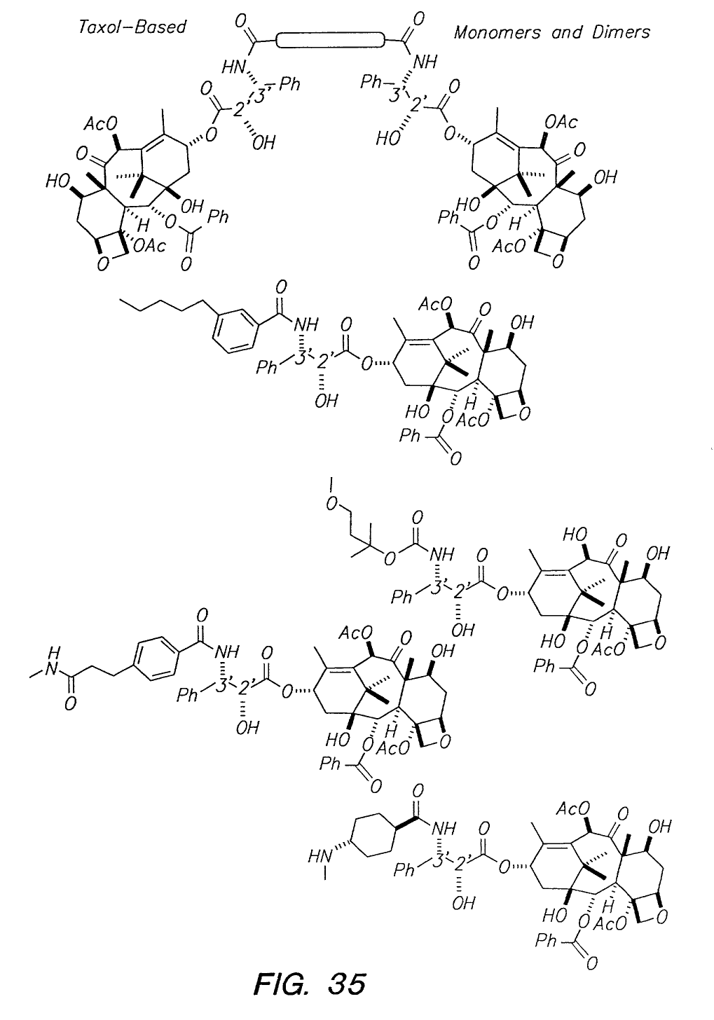

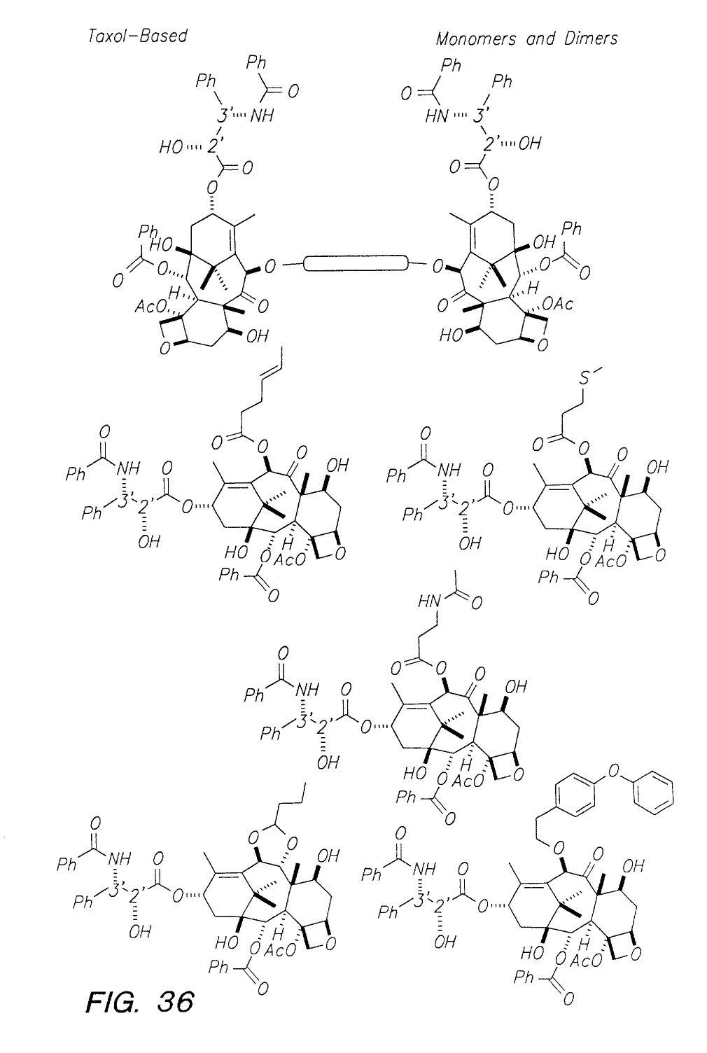

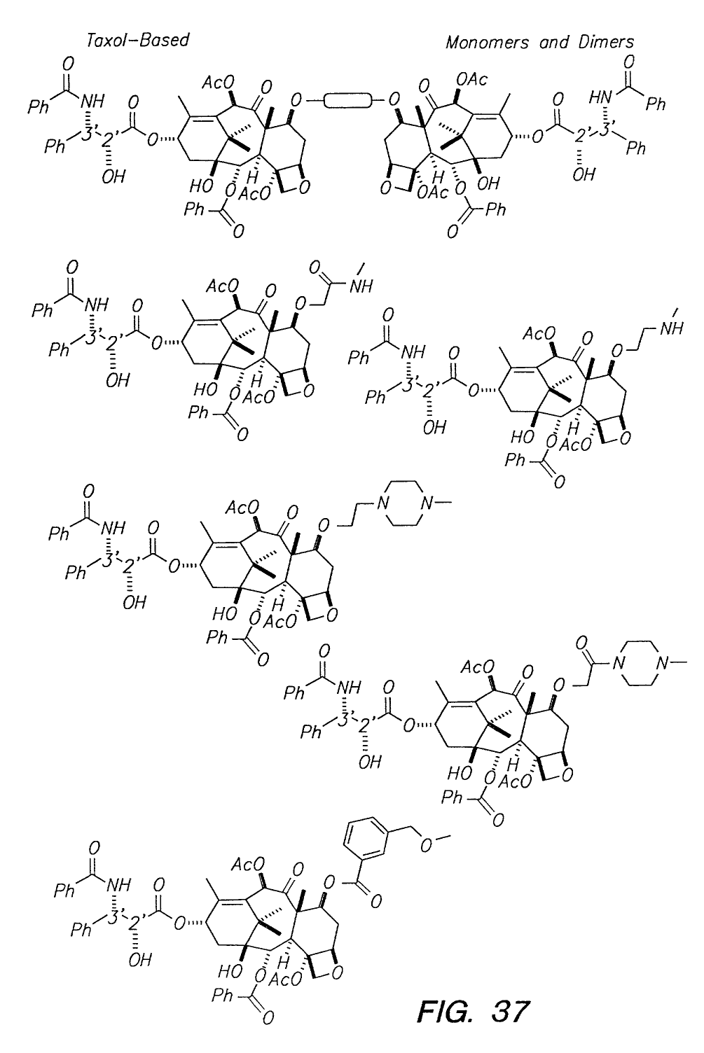

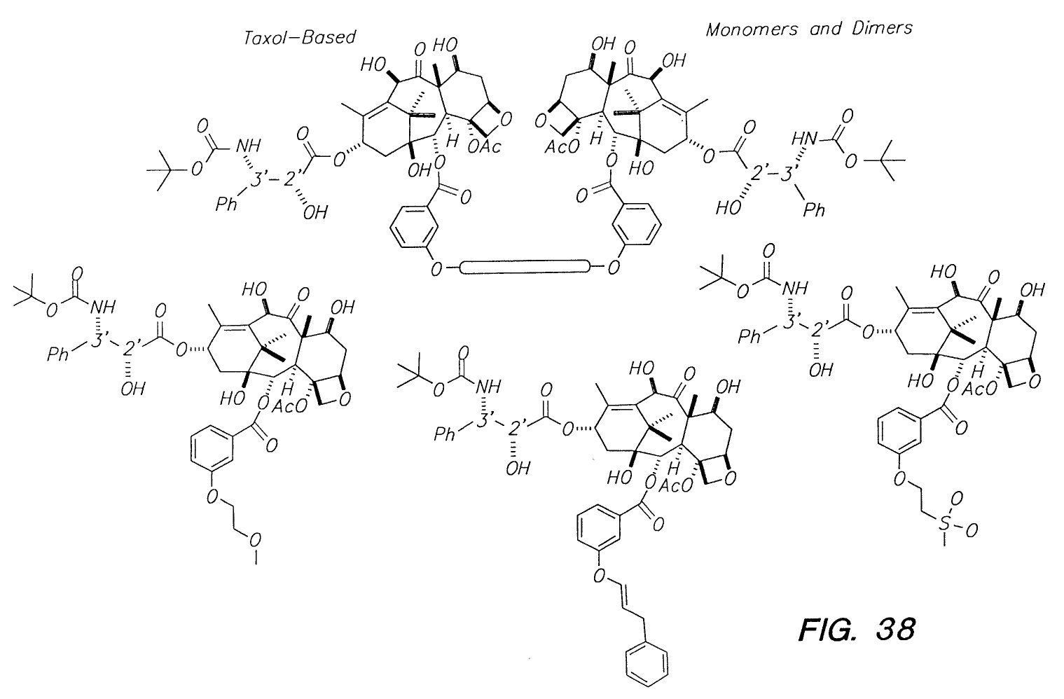

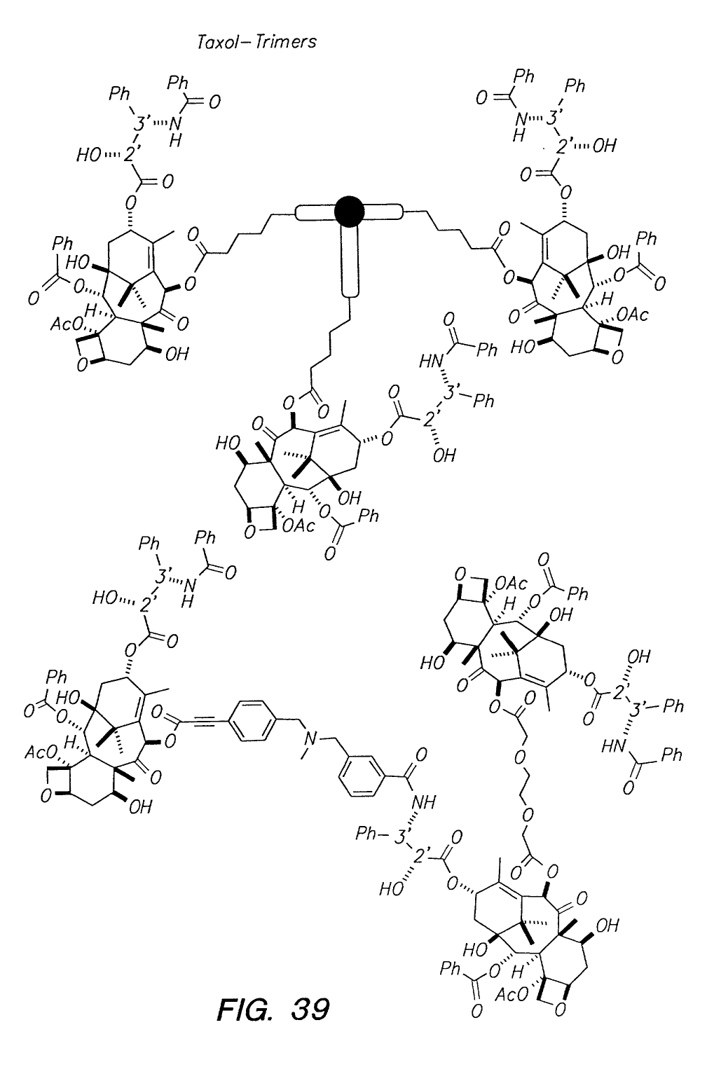

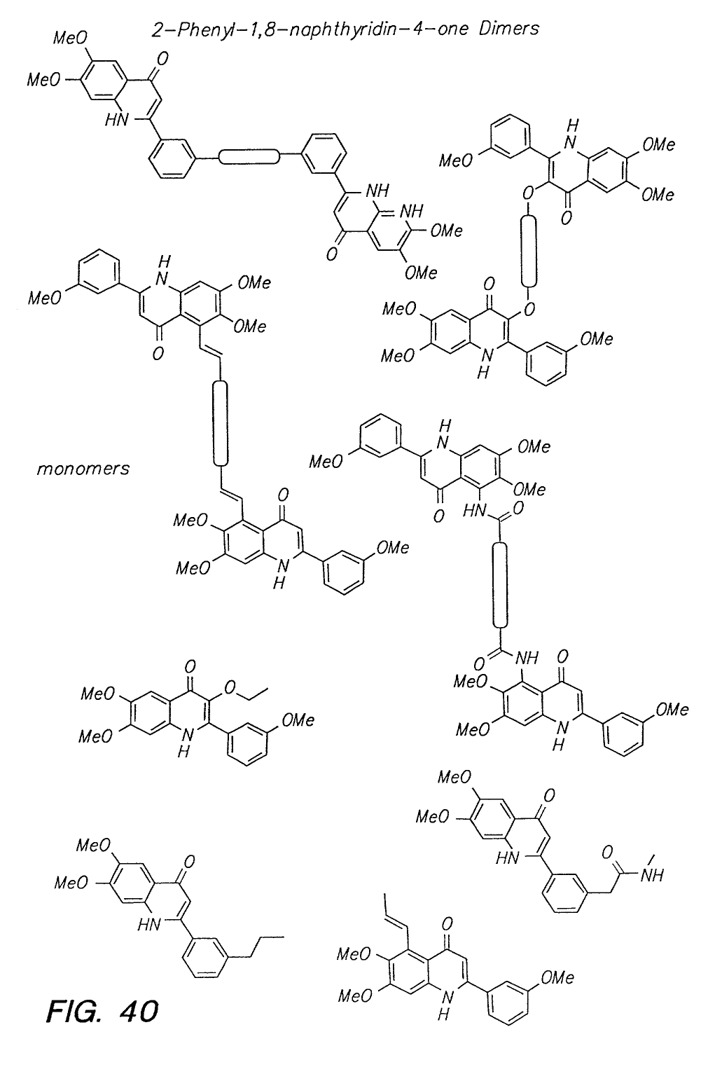

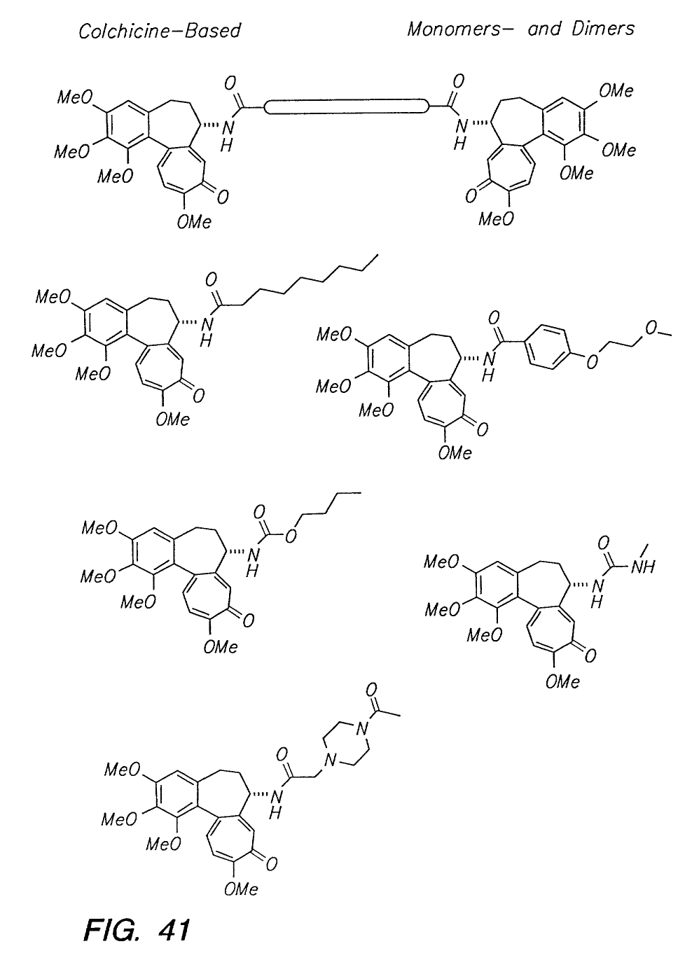

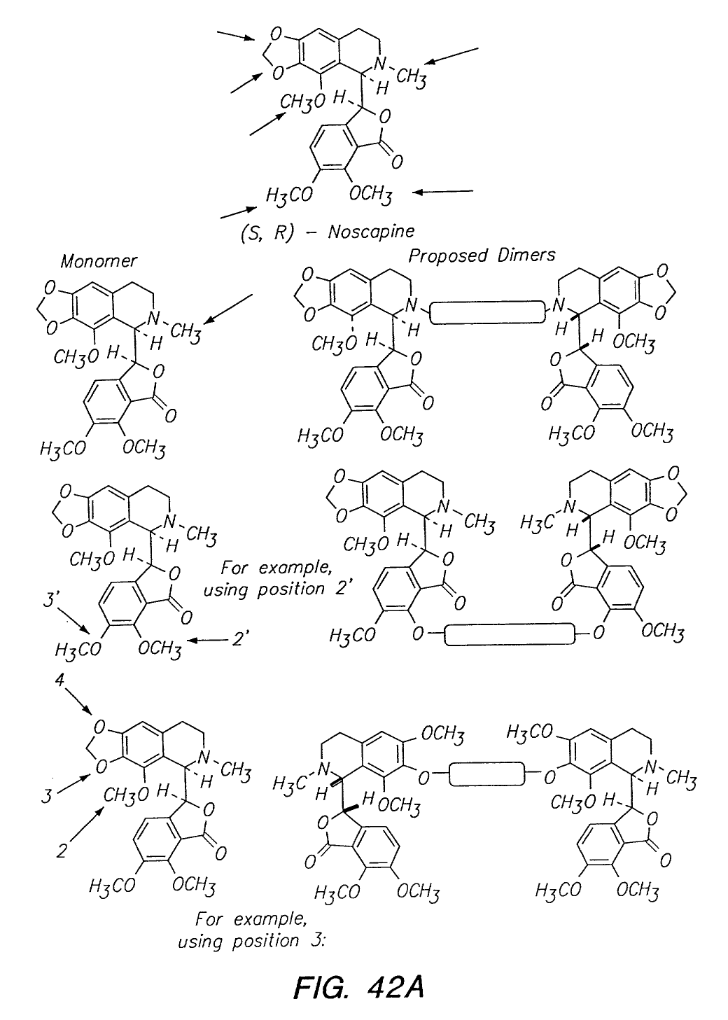

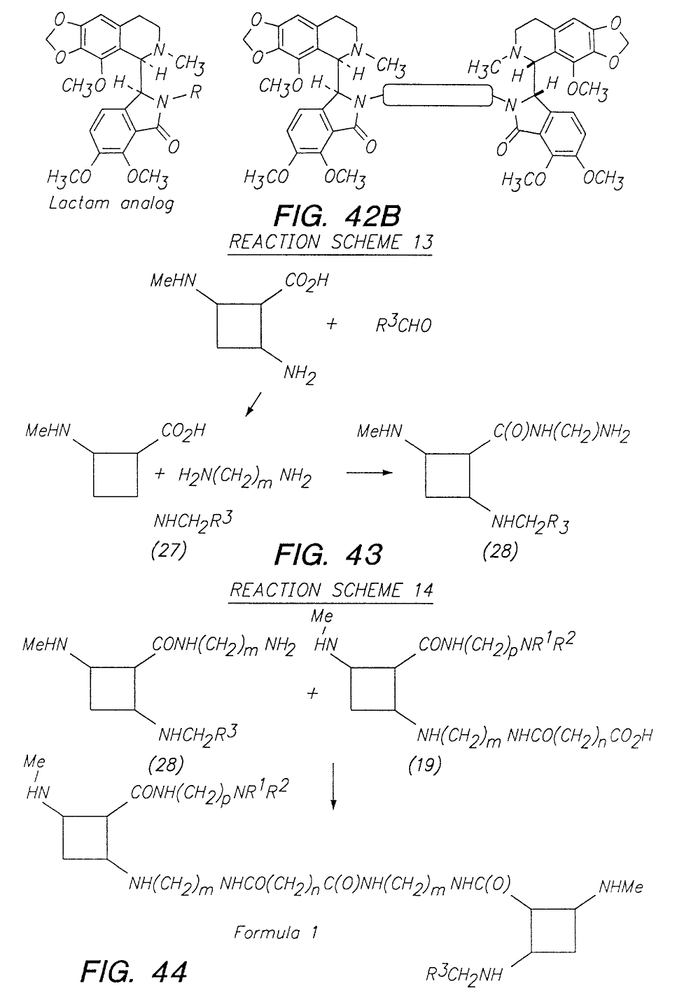

Examples of the strategies which can be used to prepare multibinding compounds based on modifiers of cellular filamentous structures are shown in FIGS. 31-32 and 34-42 attached. Specifically, FIG. 31 illustrates combrestatin dimers which can be prepared from the monomers shown. FIGS. 32 and 40 illustrate 2-phenyl-1,8-naphthyridin-4-one dimers which can be prepared from the monomers shown. FIGS. 34-39 illustrate taxol dimers and trimers which can be prepared from the monomers shown. FIG. 41 illustrate colchicine based dimers which can be prepared from the monomers shown. FIG. 42 illustrates functional points for dimerization of noscapine. Suitable dimers and monomers are also illustrated.

The basic approach involves generating monomeric ligands from known ligands that exists for a particular target mechanism. These compounds are then assayed in the primary assay to ensure that biological activity is retained. Of the extended monomeric compounds that retain activity, systematic synthesis of linkers connecting monomeric units is conducted. This linkage can be done between identical, linked monomers and between non-identical extended monomers. Linkage between different ligand classes may be desirable (for example, to reduce toxicity). This includes linking both stabilizers and destabilizers of particular interactions (for example, tubulin polymerization). The linkage itself can have desirable functions, including but not limited to, changes in pharmocokinetic, pharmocodynamic, solubility, and activity profiles. The linkage itself can include pharmacologically active components.

Isolation and Purification of the Compounds

Isolation and purification of the compounds and intermediates described herein can be effected, if desired, by any suitable separation or purification such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the Examples herein below. However, other equivalent separation or isolation procedures could, of course, also be used.

Combinatorial Libraries

The methods described above lend themselves to combinatorial approaches for identifying multimeric macromolecular compounds which possess multibinding properties.

Specifically, factors such as the properjuxtaposition of the individual macromolecular ligands of a multibinding compound with respect to the relevant array of binding sites on a macromolecular target or targets is important in optimizing the interaction of the multibinding compound with its target(s) and to maximize the biological advantage through multivalency. One approach is to identify a library of candidate multibinding macromolecular compounds with properties spanning the multibinding parameters that are relevant for a particular macromolecular target. These parameters include: (1) the identity of ligand(s), (2) the orientation of ligands, (3) the valency of the construct, (4) linker length, (5) linker geometry, (6) linker physical properties, and (7) linker chemical functional groups.

Libraries of multimeric macromolecular compounds potentially possessing multibinding properties (i.e., candidate multibinding compounds) and comprising a multiplicity of such variables are prepared and these libraries are then evaluated via conventional assays corresponding to the macromolecular ligand selected and the multibinding parameters desired. Considerations relevant to each of these variables are set forth below:

Selection of Ligand(s)

A single macromolecular ligand or set of ligands is (are) selected for incorporation into the libraries of candidate multibinding compounds which library is directed against a particular macromolecular target or targets. The only requirement for the macromolecular ligands chosen is that they are capable of interacting with the selected target(s). Thus, macromolecular ligands may be known drugs, modified forms of known drugs, substructures of known drugs or substrates of modified forms of known drugs (which are competent to interact with the target), or other compounds. Macromolecular ligands are preferably chosen based on known favorable properties that may be projected to be carried over to or amplified in multibinding forms. Favorable properties include demonstrated safety and efficacy in human patients, appropriate PK/ADME profiles, synthetic accessibility, and desirable physical properties such as solubility, logP, etc. However, it is crucial to note that macromolecular ligands which display an unfavorable property from among the previous list may obtain a more favorable property through the process of multibinding compound formation; i.e., macromolecular ligands should not necessarily be excluded on such a basis. For example, a macromolecular ligand that is not sufficiently potent at a particular macromolecular target so as to be efficacious in a human patient may become highly potent and efficacious when presented in multibinding form. A macromolecular ligand that is potent and efficacious but not of utility because of a non-mechanism-related toxic side effect may have increased therapeutic index (increased potency relative to toxicity) as a multibinding compound. Compounds that exhibit short in vivo half-lives may have extended half-lives as multibinding compounds. Physical properties of macromolecular ligands that limit their usefulness (e.g. poor bioavailability due to low solubility, hydrophobicity, hydrophilicity) may be rationally modulated in multibinding forms, providing compounds with physical properties consistent with the desired utility.

Orientation: Selection of Ligand Attachment Points and Linking Chemistry

Several points are chosen on each macromolecular ligand at which to attach the ligand to the linker. The selected points on the ligand/linker for attachment are functionalized to contain complementary reactive functional groups. This permits probing the effects of presenting the ligands to their receptor(s) in multiple relative orientations, an important multibinding design parameter. The only requirement for choosing attachment points is that attaching to at least one of these points does not abrogate activity of the ligand. Such points for attachment can be identified by structural information when available. For example, inspection of a co-crystal structure of a protease inhibitor bound to its target allows one to identify one or more sites where linker attachment will not preclude the enzyme:inhibitor interaction. Alternatively, evaluation of ligand/target binding by nuclear magnetic resonance will permit the identification of sites non-essential for ligand/target binding. See, for example, Fesik, et al., U.S. Pat. No. 5,891,643. When such structural information is not available, utilization of structure-activity relationships (SAR) for ligands will suggest positions where substantial structural variations are and are not allowed. In the absence of both structural and SAR information, a library is merely selected with multiple points of attachment to allow presentation of the ligand in multiple distinct orientations. Subsequent evaluation of this library will indicate what positions are suitable for attachment.

It is important to emphasize that positions of attachment that do abrogate the activity of the monomeric macromolecular ligand may also be advantageously included in candidate multibinding compounds in the library provided that such compounds bear at least one ligand attached in a manner which does not abrogate intrinsic activity. This selection derives from, for example, heterobivalent interactions within the context of a single target molecule. For example, consider a receptor antagonist ligand bound to its target receptor, and then consider modifying this ligand by attaching to it a second copy of the same ligand with a linker which allows the second ligand to interact with the same receptor molecule at sites proximal to the antagonist binding site, which include elements of the receptor that are not part of the formal antagonist binding site and/or elements of the matrix surrounding the receptor such as the membrane. Here, the most favorable orientation for interaction of the second ligand molecule with the receptor/matrix may be achieved by attaching it to the linker at a position which abrogates activity of the ligand at the formal antagonist binding site. Another way to consider this is that the SAR of individual ligands within the context of a multibinding structure is often different from the SAR of those same ligands in momomeric form.

The foregoing discussion focused on bivalent interactions of dimeric compounds bearing two copies of the same ligand joined to a single linker through different attachment points, one of which may abrogate the binding/activity of the monomeric ligand. It should also be understood that bivalent advantage may also be attained with heterodimeric constructs bearing two different ligands that bind to common or different targets.

Once the macromolecular ligand attachment points have been chosen, one identifies the types of chemical linkages that are possible at those points. The most preferred types of chemical linkages are those that are compatible with the overall structure of the ligand (or protected forms of the ligand) readily and generally formed, stable and intrinsically inocuous under typical chemical and physiological conditions, and compatible with a large number of available linkers. Amide bonds, ethers, amines, carbamates, ureas, and sulfonamides are but a few examples of preferred linkages.

Linkers: Spanning Relevant Multibinding Parameters Through Selection of Valency, Linker Length, Linker Geometry, Rigidity, Physical Properties, and Chemical Functional Groups

In the library of linkers employed to generate the library of candidate multibinding compounds, the selection of linkers employed in this library of linkers takes into consideration the following factors:

Valency. In most instances the library of linkers is initiated with divalent linkers. The choice of ligands and proper juxtaposition of two ligands relative to their binding sites permits such molecules to exhibit target binding affinities and specificities more than sufficient to confer biological advantage. Furthermore, divalent linkers or constructs are also typically of modest size such that they retain the desirable biodistribution properties of small molecules.

Linker length. Linkers are chosen in a range of lengths to allow the spanning of a range of inter-ligand distances that encompass the distance preferable for a given divalent interaction. In some instances the preferred distance can be estimated rather precisely from high-resolution structural information of targets, typically enzymes and soluble receptor targets. In other instances where high-resolution structural information is not available (such as 7TM G-protein coupled receptors), one can make use of simple models to estimate the maximum distance between binding sites either on adjacent receptors or at different locations on the same receptor. In situations where two binding sites are present on the same target (or target subunit for multisubunit targets), preferred linker distances are 2-20 Å, with more preferred linker distances of 3-12 Å. In situations where two binding sites reside on separate (e.g., protein) target sites, preferred linker distances are 20-100 Å, with more preferred distances of 30-70 Å.

Linker geometry and rigidity. The combination of ligand attachment site, linker length, linker geometry, and linker rigidity determine the possible ways in which the ligands of candidate multibinding compounds may be displayed in three dimensions and thereby presented to their binding sites. Linker geometry and rigidity are nominally determined by chemical composition and bonding pattern, which may be controlled and are systematically varied as another spanning function in a multibinding array. For example, linker geometry is varied by attaching two ligands to the ortho, meta, and para positions of a benzene ring, or in cis- or trans-arrangements at the 1,1-vs. 1,2-vs. 1,3-vs. 1,4-positions around a cyclohexane core or in cis- or trans-arrangements at a point of ethylene unsaturation. Linker rigidity is varied by controlling the number and relative energies of different conformational states possible for the linker. For example, a divalent compound bearing two ligands joined by 1,8-octyl linker has many more degrees of freedom, and is therefore less rigid than a compound in which the two ligands are attached to the 4,4′ positions of a biphenyl linker.

Linker physical properties. The physical properties of linkers are nominally determined by the chemical constitution and bonding patterns of the linker, and linker physical properties impact the overall physical properties of the candidate multibinding compounds in which they are included. A range of linker compositions is typically selected to provide a range of physical properties (hydrophobicity, hydrophilicity, amphiphilicity, polarization, acidity, and basicity) in the candidate multibinding compounds. The particular choice of linker physical properties is made within the context of the physical properties of the ligands they join and preferably the goal is to generate molecules with favorable PK/ADME properties. For example, linkers can be selected to avoid those that are too hydrophilic or too hydrophobic to be readily absorbed and/or distributed in vivo.

Linker chemical functional groups. Linker chemical functional groups are selected to be compatible with the chemistry chosen to connect linkers to the ligands and to impart the range of physical properties sufficient to span initial examination of this parameter.

Combinatorial Synthesis

Having chosen a set of n macromolecular ligands (n being determined by the sum of the number of different attachment points for each ligand chosen) and m linkers by the process outlined above, a library of (n!)m candidate divalent multibinding macromolecular compounds is prepared which spans the relevant multibinding design parameters for a particular macromolecular target. For example, an array generated from two macromolecular ligands, one which has two attachment points (A1, A2) and one which has three attachment points (B1, B2, B3) joined in all possible combinations provide for at least 15 possible combinations of multibinding compounds:

| | | | | | | | |
|---|---|---|---|---|---|---|---|
| A1-A1 | A1-A2 | A1-B1 | A1-B2 | A1-B3 | A2-A2 | A2-B1 | A2- |
| | | | | | | | B2 |
| A2-B3 | B1-B1 | B1-B2 | B1-B3 | B2-B2 | B2-B3 | B3-B3 | |

When each of these combinations is joined by 10 different linkers, a library of 150 candidate multibinding compounds results.

Given the combinatorial nature of the library, common chemistries are preferably used to join the reactive functionalies on the ligands with complementary reactive functionalities on the linkers. The library therefore lends itself to efficient parallel synthetic methods. The combinatorial library can employ solid phase chemistries well known in the art wherein the ligand and/or linker is attached to a solid support. Alternatively and preferably, the combinatorial libary is prepared in the solution phase. After synthesis, candidate multibinding compounds are optionally purified before assaying for activity by, for example, chromatographic methods (e.g., HPLC).

Analysis of Array by Biochemical, Analytical, Pharmacological, and Computational Methods

Various methods are used to characterize the properties and activities of the candidate multibinding compounds in the library to determine which compounds possess multibinding properties. Physical constants such as solubility under various solvent conditions and log D/c log D values can be determined. A combination of NMR spectroscopy and computational methods is used to determine low-energy conformations of the candidate multibinding compounds in fluid media. The ability of the members of the library to bind to the desired target and other targets is determined by various standard methods, which include radioligand displacement assays for receptor and ion channel targets, and kinetic inhibition analysis for many enzyme targets. In vitro efficacy, such as for receptor agonists and antagonists, ion channel blockers, and antimicrobial activity, can also be determined. Pharmacological data, including oral absorption, everted gut penetration, other pharmacokinetic parameters and efficacy data can be determined in appropriate models. In this way, key structure-activity relationships are obtained for multibinding design parameters which are then used to direct future work.

The members of the library which exhibit multibinding properties, as defined herein, can be readily determined by conventional methods. First those members which exhibit multibinding properties are identified by conventional methods as described above including conventional assays (both in vitro and in vivo).

Second, ascertaining the structure of those compounds which exhibit multibinding properties can be accomplished via art recognized procedures. For example, each member of the library can be encrypted or tagged with appropriate information allowing determination of the structure of relevant members at a later time. See, for example, Dower, et al., International Patent Application Publication No. WO 93/06121; Brenner, et al., Proc. Natl. Acad. Sci., USA, 89:5181 (1992); Gallop, et al., U.S. Pat. No. 5,846,839; each of which are incorporated herein by reference in its entirety. Alternatively, the structure of relevant multivalent compounds can also be determined from soluble and untagged libaries of candidate multivalent compounds by methods known in the art such as those described by Hindsgaul, et al., Canadian Patent Application No. 2,240,325 which was published on Jul. 11, 1998. Such methods couple frontal affinity chromatography with mass spectroscopy to determine both the structure and relative binding affinities of candidate multibinding compounds to receptors.

The process set forth above for dimeric candidate multibinding compounds can, of course, be extended to trimeric candidate compounds and higher analogs thereof.

Follow-up Synthesis and Analysis of Additional Array(s)

Based on the information obtained through analysis of the initial library, an optional component of the process is to ascertain one or more promising multibinding “lead” compounds as defined by particular relative ligand orientations, linker lengths, linker geometries, etc. Additional libraries can then be generated around these leads to provide for further information regarding structure to activity relationships. These arrays typically bear more focused variations in linker structure in an effort to further optimize target affinity and/or activity at the target (antagonism, partial agonism, etc.), and/or alter physical properties. By iterative redesign/analysis using the novel principles of multibinding design along with classical medicinal chemistry, biochemistry, and pharmacology approaches, one is able to prepare and identify optimal multibinding compounds that exhibit biological advantage towards their targets and as therapeutic agents.

To further elaborate upon this procedure, suitable divalent linkers include, by way of example only, those derived from dicarboxylic acids, disulfonylhalides, dialdehydes, diketones, dihalides, diisocyanates,diamines, diols, mixtures of carboxylic acids, sulfonylhalides, aldehydes, ketones, halides, isocyanates, amines and diols. In each case, the carboxylic acid, sulfonylhalide, aldehyde, ketone, halide, isocyanate, amine and diol functional group is reacted with a complementary functionality on the ligand to form a covalent linkage. Such complementary functionality is well known in the art as illustrated in the following table:

COMPLEMENTARY BINDIING CHEMISTRIES

| First Reactive Group | Second Reactive Group | Linkage |
|---|---|---|
| hydroxyl | isocyanate | urethane |
| amine | epoxide | β-hydroxyamine |
| sulfonyl halide | amine | sulfonamide |
| carboxyl acid | amine | amide |
| hydroxyl | alkyl/aryl halide | ether |
| aldehyde | amine/NaCNBH₄ | amine |
| ketone | amine/NaCNBH₄ | amine |
| amine | isocyanate | urea |

Exemplary linkers include the following linkers identified as X-1 through X-418 as set forth below:

Diacids

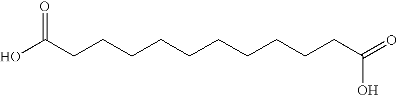

X-1

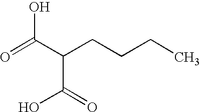

X-2

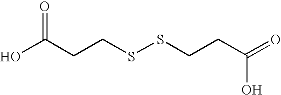

X-3

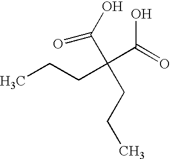

X-4

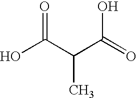

X-5

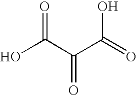

X-6

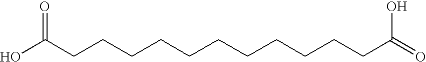

X-7

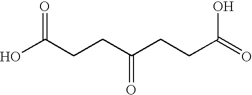

X-8

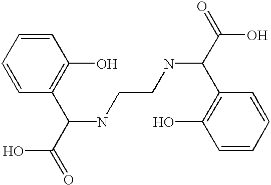

X-9

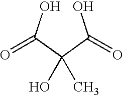

X-10

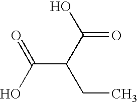

X-11

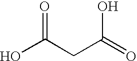

X-12

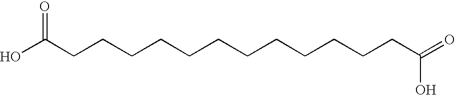

X-13

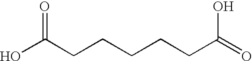

X-14

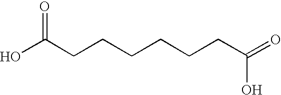

X-15

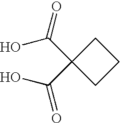

X-16

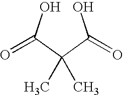

X-17

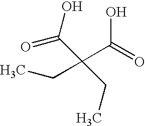

X-18

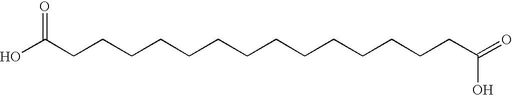

X-19

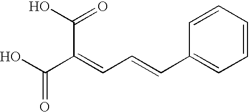

X-20

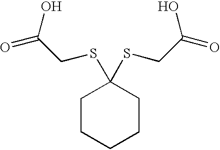

X-21

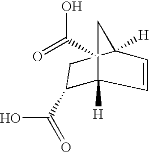

X-22

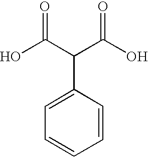

X-23

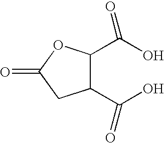

X-24

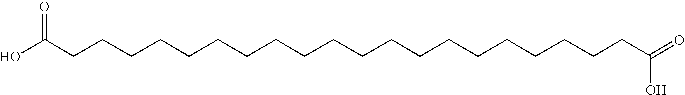

X-25

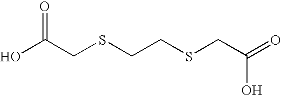

X-26

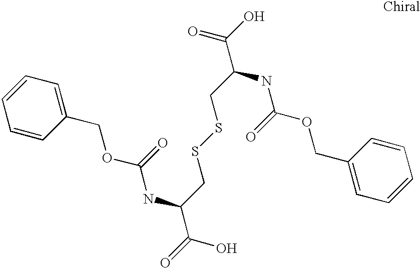

X-27

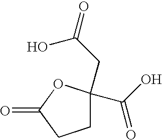

X-28

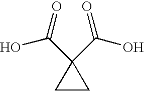

X-29

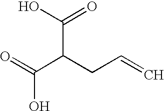

X-30

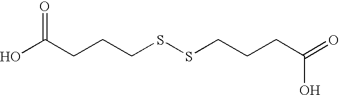

X-31

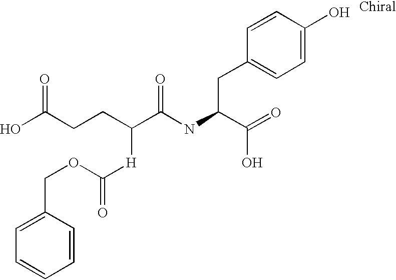

X-32

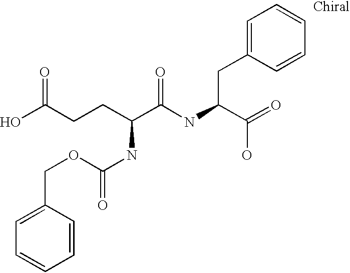

X-33

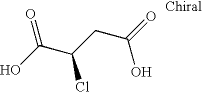

X-34

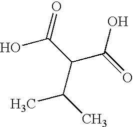

X-35

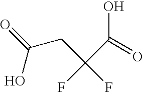

X-36

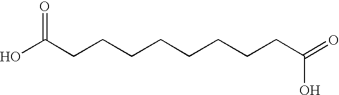

X-37

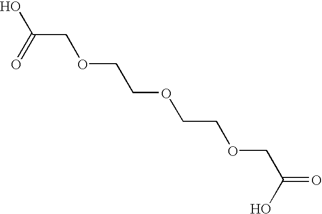

X-38

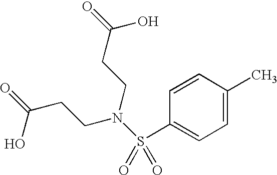

X-39

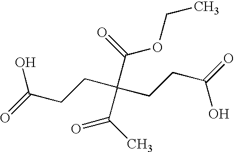

X-40

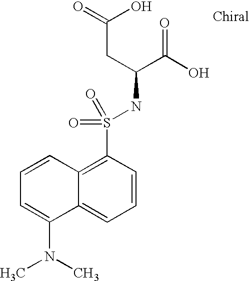

X-41

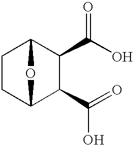

X-42

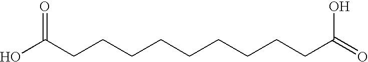

X-43

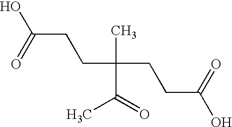

X-44

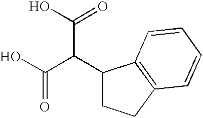

X-45

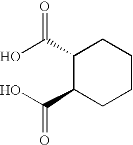

X-46

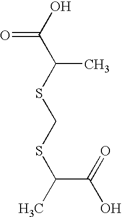

X-47

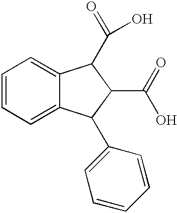

X-48

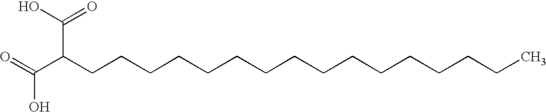

X-49

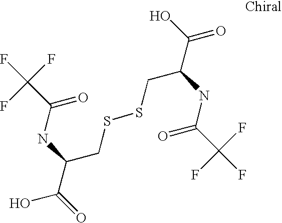

X-50

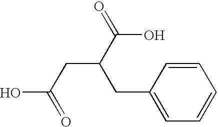

X-51

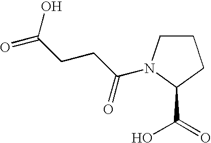

X-52

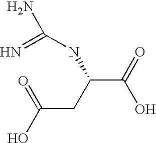

X-53

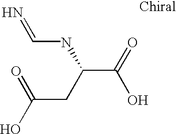

X-54

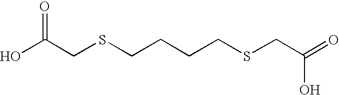

X-55

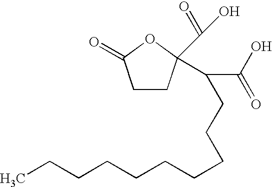

X-56

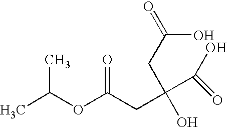

X-57

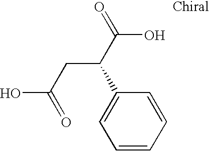

X-58

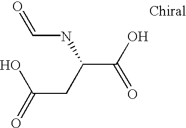

X-59

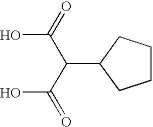

X-60

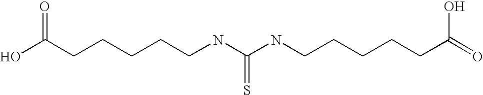

X-61

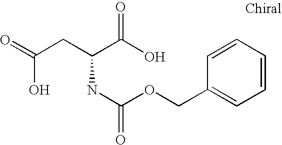

X-62

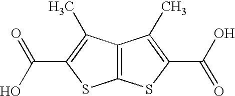

X-63

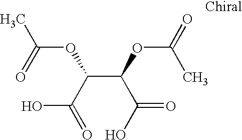

X-64

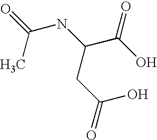

X-65

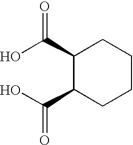

X-66

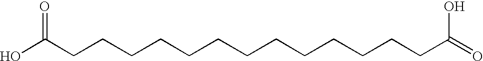

X-67

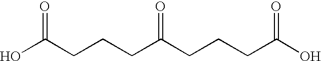

X-68

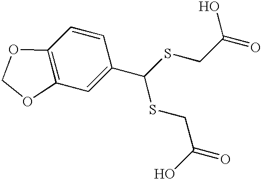

X-69

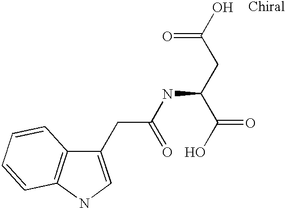

X-70

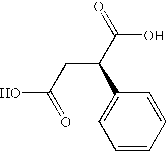

X-71

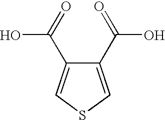

X-72

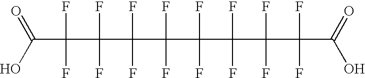

X-73

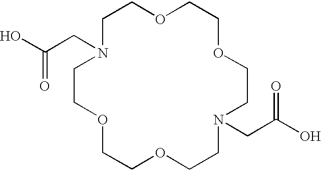

X-74

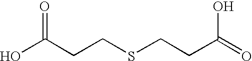

X-75

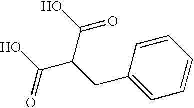

X-76

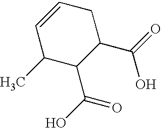

X-77

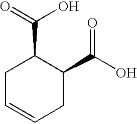

X-78

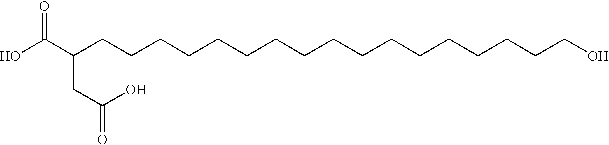

X-79

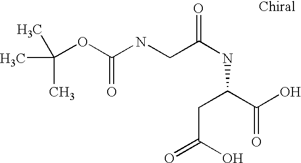

X-80

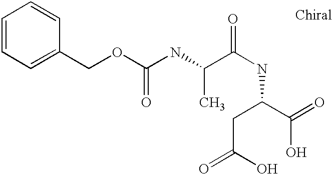

X-81

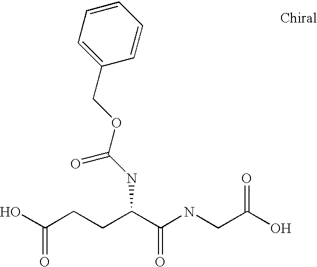

X-82

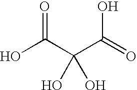

X-83

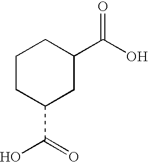

X-84

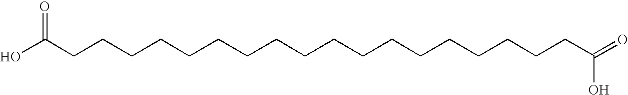

X-85

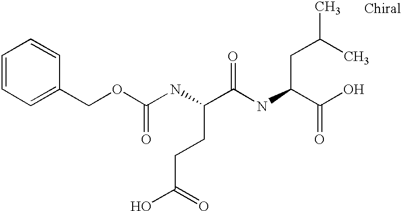

X-86

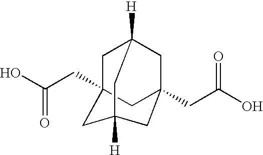

X-87

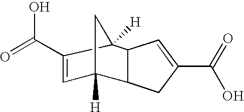

X-88

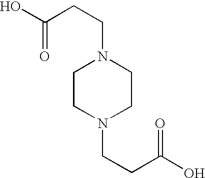

X-89

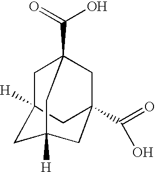

X-90

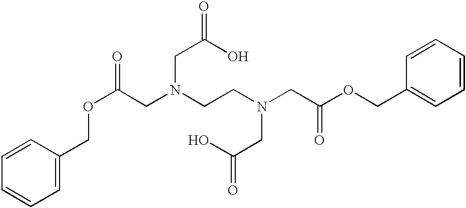

X-91

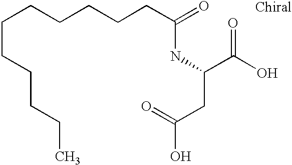

X-92

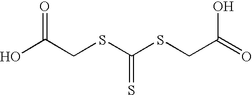

X-93

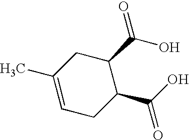

X-94

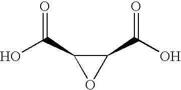

X-95

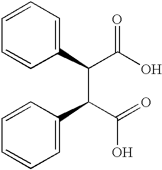

X-96

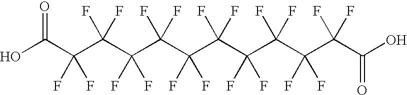

X-97

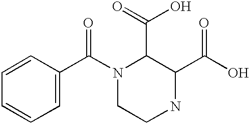

X-98

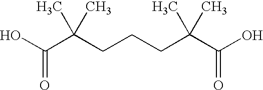

X-99

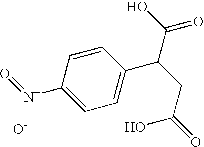

X-100

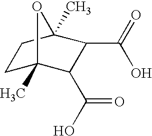

X-101

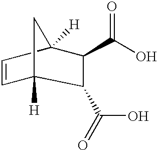

X-102

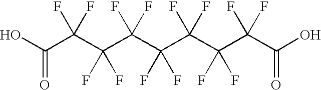

X-103

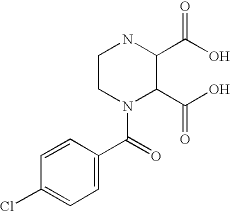

X-104

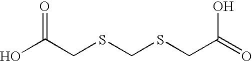

X-105

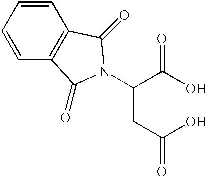

X-106

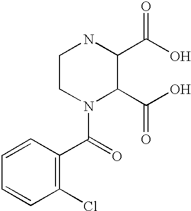

X-107

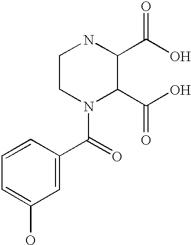

X-108

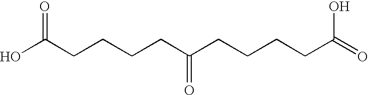

X-109

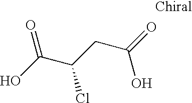

X-110

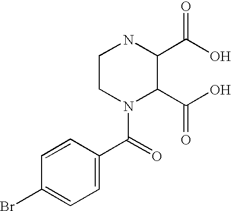

X-111

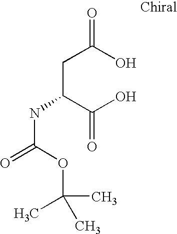

X-112

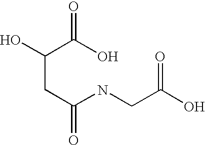

X-113

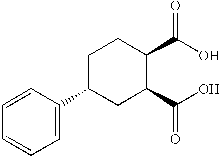

X-114

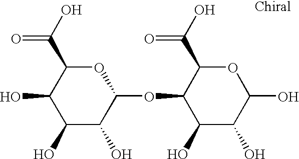

X-115

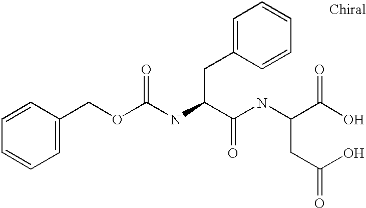

X-116

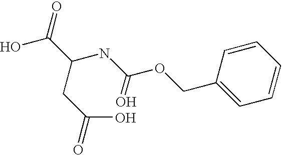

X-117

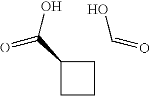

X-118

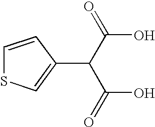

X-119

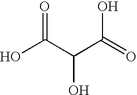

X-120

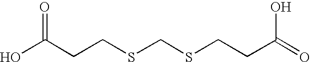

X-121

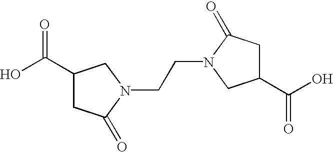

X-122

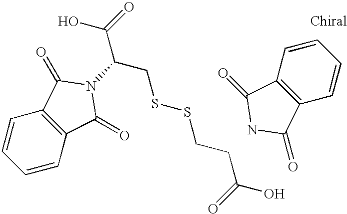

X-123

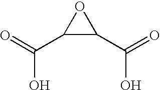

X-124

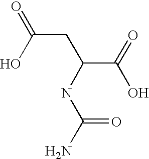

X-125

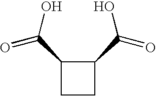

X-126

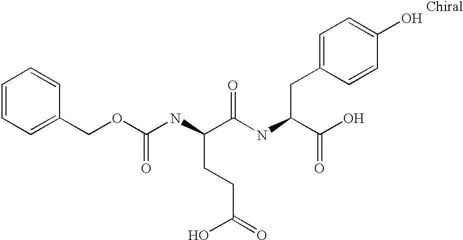

X-127

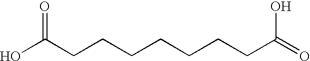

X-128

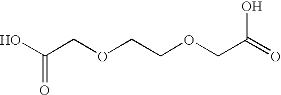

X-129

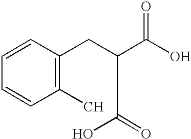

X-130

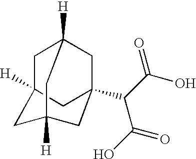

X-131

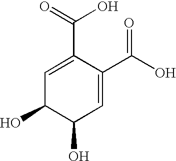

X-132
Disulfonyl Halides

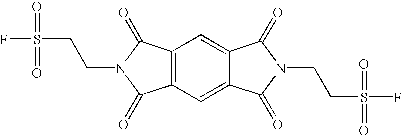

X-133

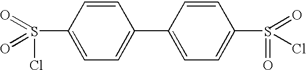

X-134

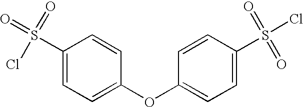

X-135

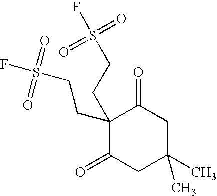

X-136

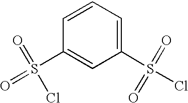

X-137

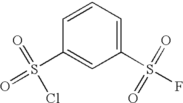

X-138

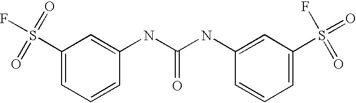

X-139

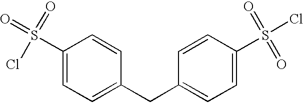

X-140

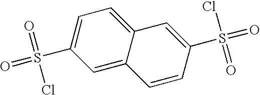

X-141

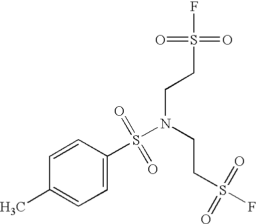

X-142

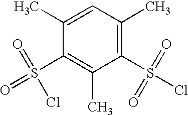

X-143

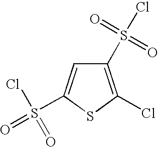

X-144

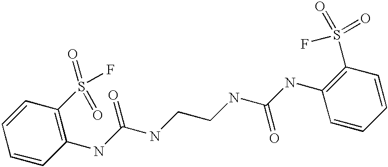

X-145

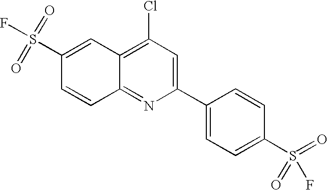

X-146

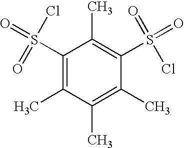

X-147

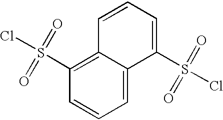

X-148

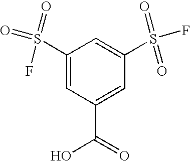

X-149

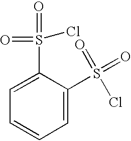

X-150

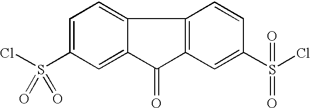

X-151

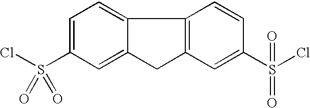

X-152
Dialdehydes

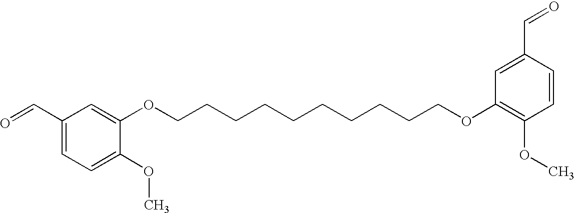

X-153

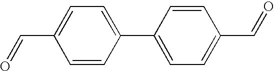

X-154

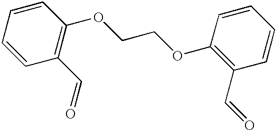

X-155

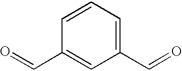

X-156

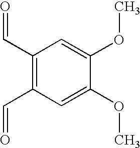

X-157

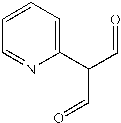

X-158

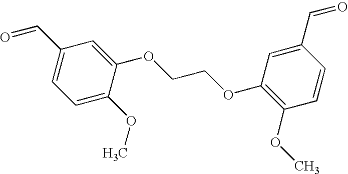

X-159

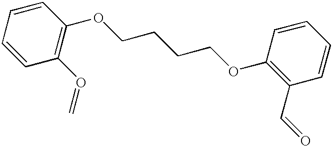

X-160

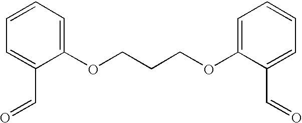

X-161

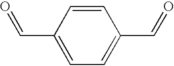

X-162

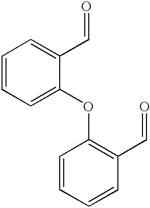

X-163

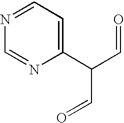

X-164

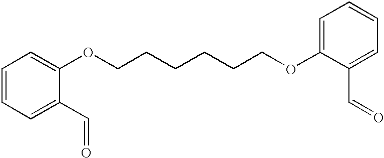

X-165

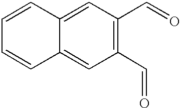

X-166

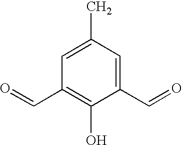

X-167

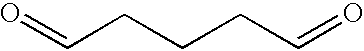

X-168

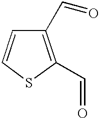

X-169

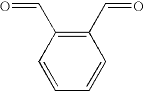

X-170

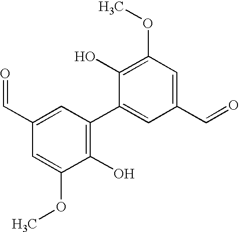

X-171

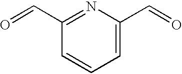

X-172

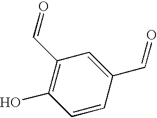

X-173

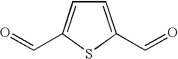

X-174
Dihalides

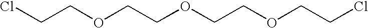

X-175

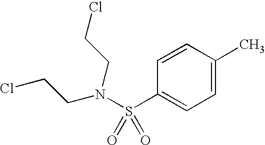

X-176

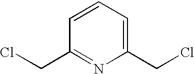

X-177

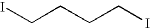

X-178

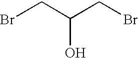

X-179

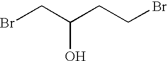

X-180

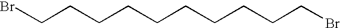

X-181

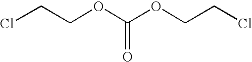

X-182

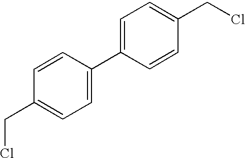

X-183

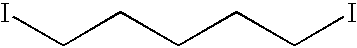

X-184

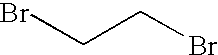

X-185

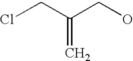

X-186

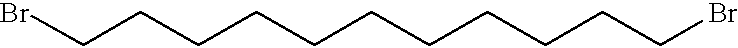

X-187

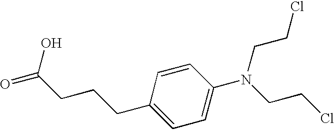

X-188

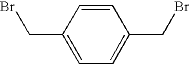

X-189

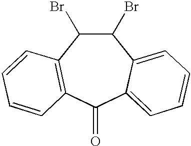

X-190

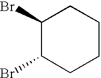

X-191

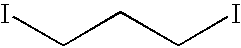

X-192

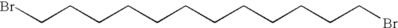

X-193

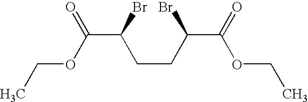

X-194

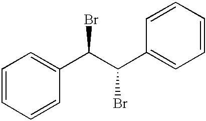

X-195

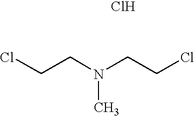

X-196

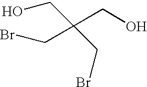

X-197

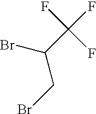

X-198

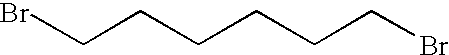

X-199

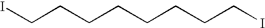

X-200

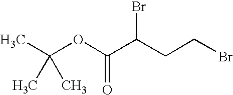

X-201

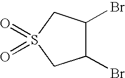

X-202

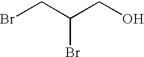

X-203

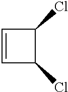

X-204

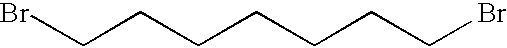

X-205

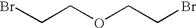

X-206

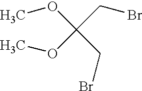

X-207

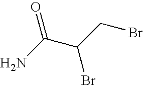

X-208

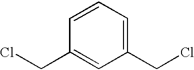

X-209

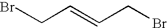

X-210

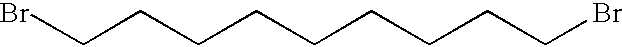

X-211

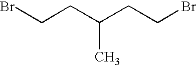

X-212

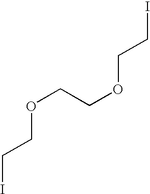

X-213

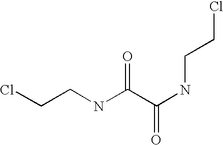

X-214
Diisocyanates

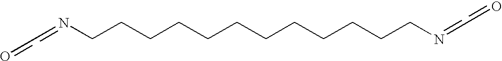

X-215

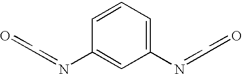

X-216

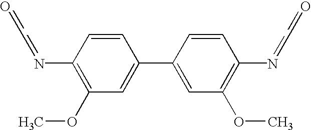

X-217

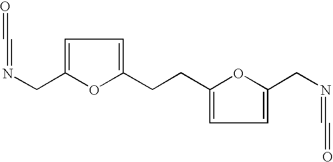

X-218

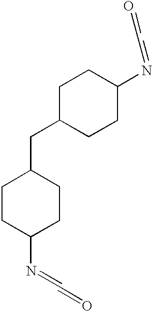

X-219

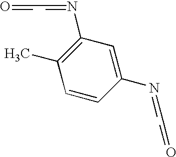

X-220

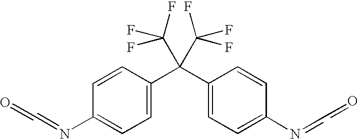

X-221

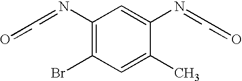

X-222

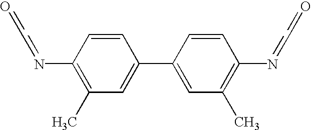

X-223

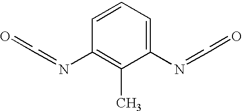

X-224

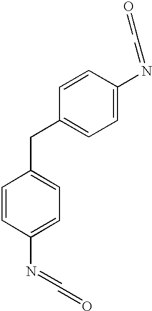

X-225

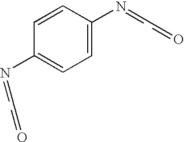

X-226

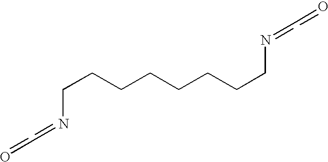

X-227

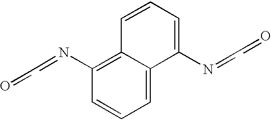

X-228

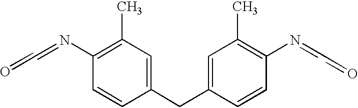

X-229

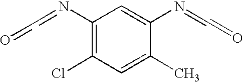

X-230

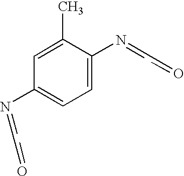

X-231

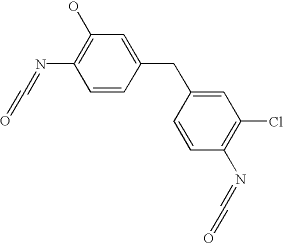

X-232

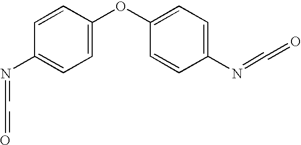

X-233

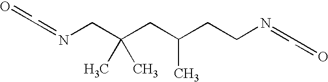

X-234

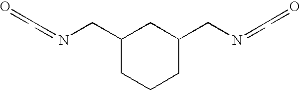

X-235

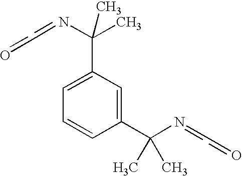

X-236

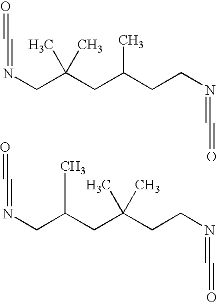

X-237

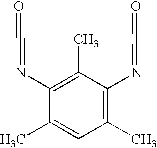

X-238

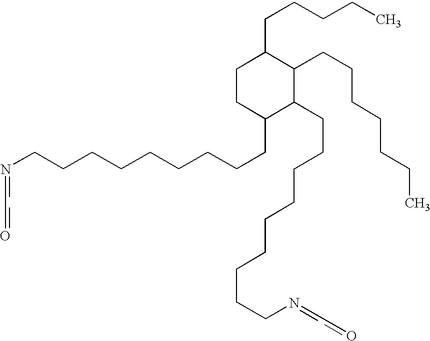

X-239

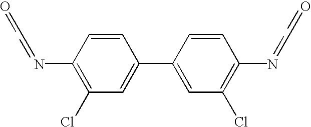

X-240

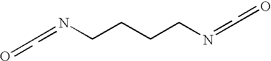

X-241

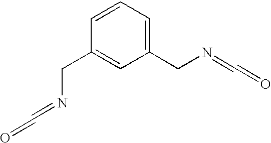

X-242

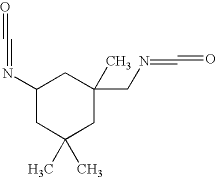

X-243

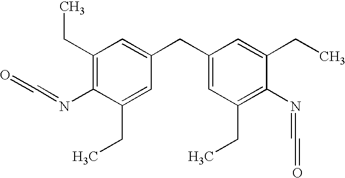

X-244

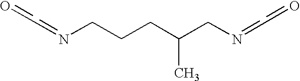

X-245

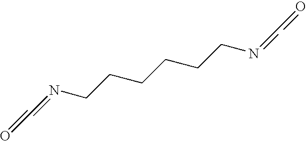

X-246

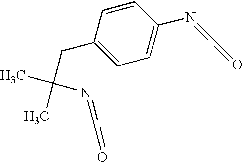

X-247

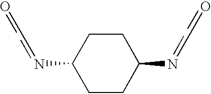

X-248
Diamines

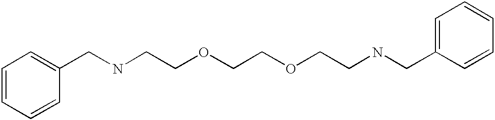

X-249

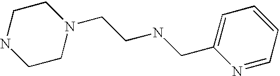

X-250

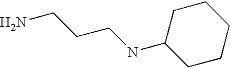

X-251

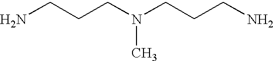

X-252

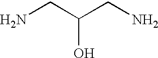

X-253

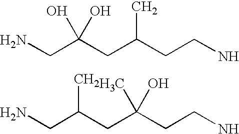

X-254

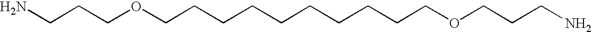

X-255

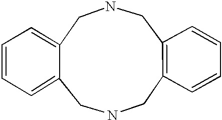

X-256

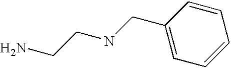

X-257

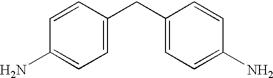

X-258

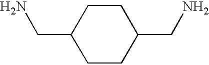

X-259

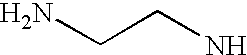

X-260

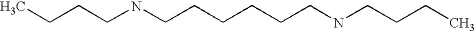

X-261

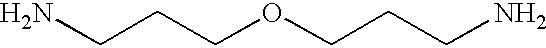

X-262

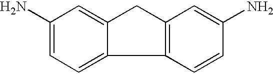

X-263

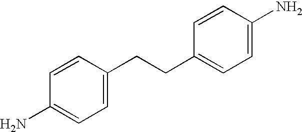

X-264

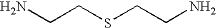

X-265

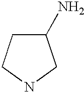

X-266

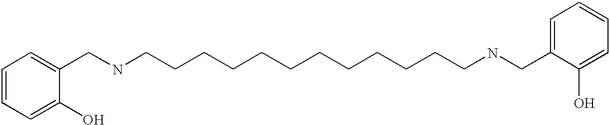

X-267

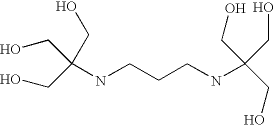

X-268

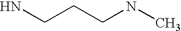

X-269

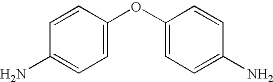

X-270

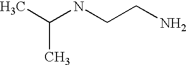

X-271

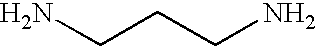

X-272

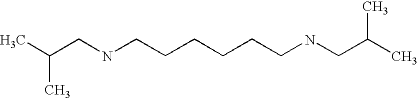

X-273

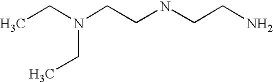

X-274

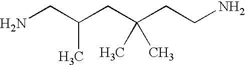

X-275

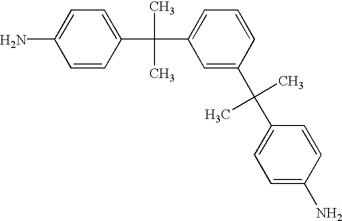

X-276

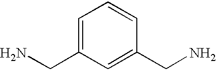

X-277

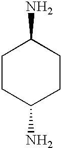

X-278

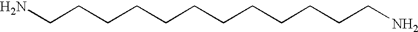

X-279

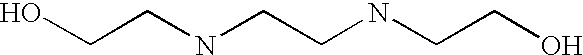

X-280

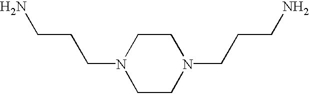

X-281

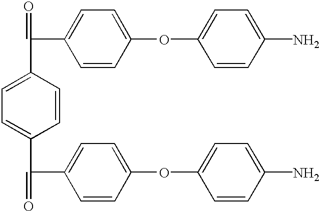

X-282

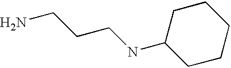

X-283

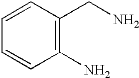

X-284

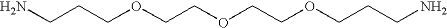

X-285

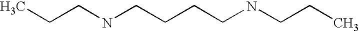

X-286

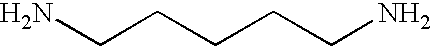

X-287

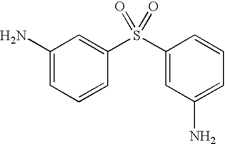

X-288

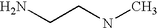

X-289

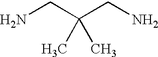

X-290

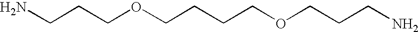

X-291

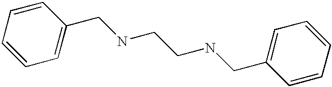

X-292

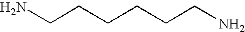

X-293

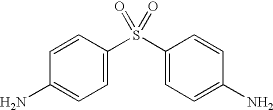

X-294

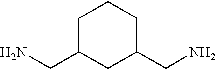

X-295

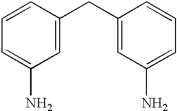

X-296

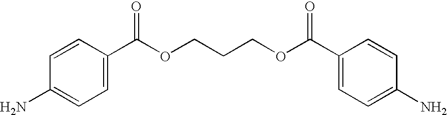

X-297

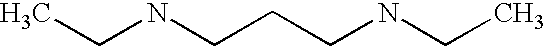

X-298

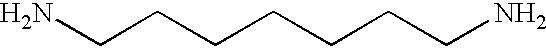

X-299

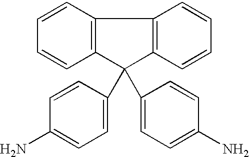

X-300

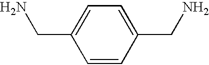

X-301

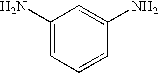

X-302

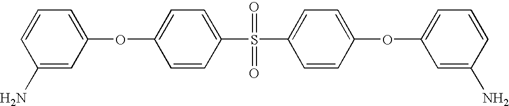

X-303

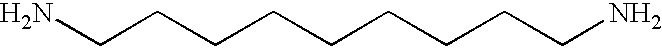

X-304

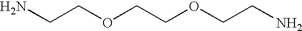

X-305

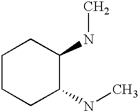

X-306

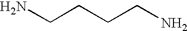

X-307

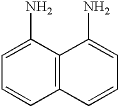

X-308

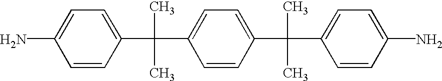

X-309

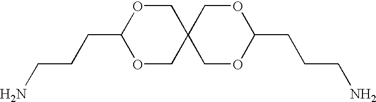

X-310

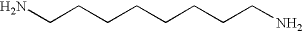

X-311

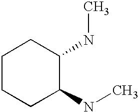

X-312

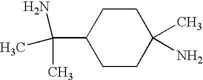

X-313

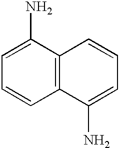

X-314

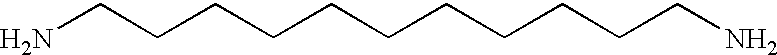

X-315

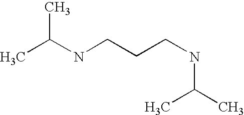

X-316

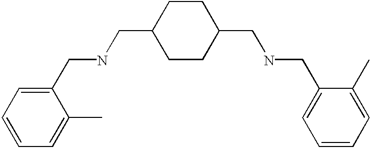

X-317

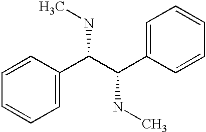

X-318

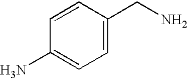

X-319

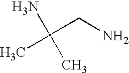

X-320

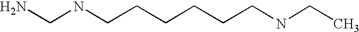

X-321

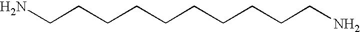

X-322

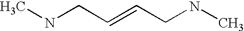

X-323

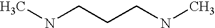

X-324

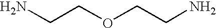

X-325
Diols

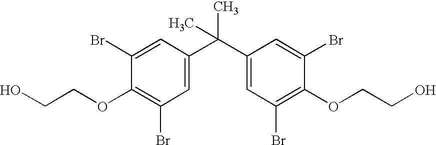

X-326

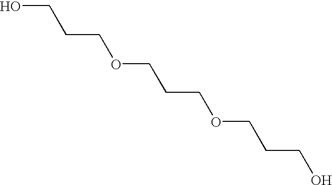

X-327

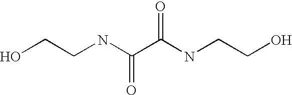

X-328

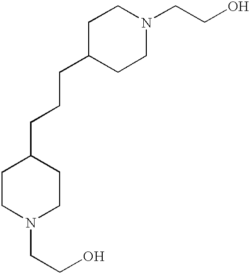

X-329

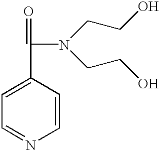

X-330

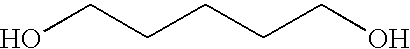

X-331

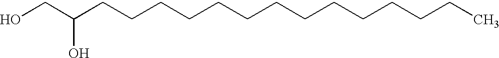

X-332

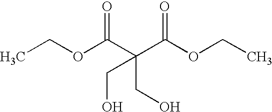

X-333

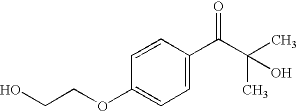

X-334

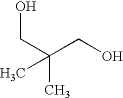

X-335

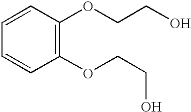

X-336

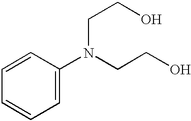

X-337

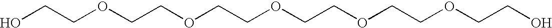

X-338

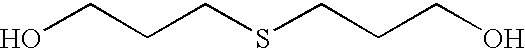

X-339

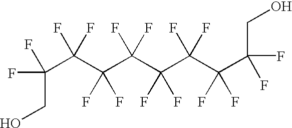

X-340

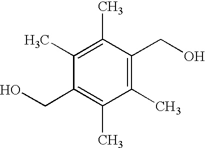

X-341

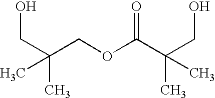

X-342

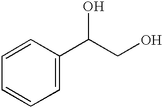

X-343

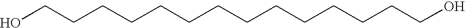

X-344

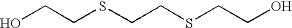

X-345

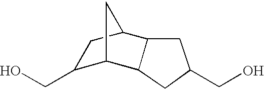

X-346

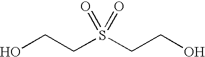

X-347

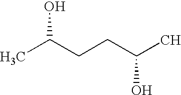

X-348

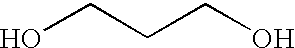

X-349

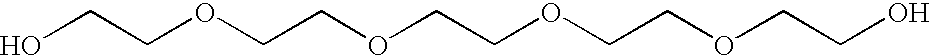

X-350

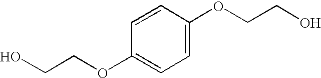

X-351

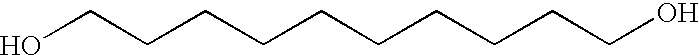

X-352

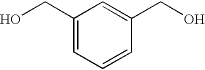

X-353

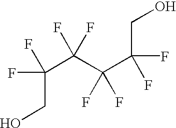

X-354

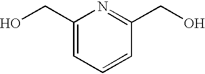

X-355

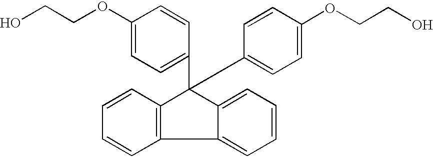

X-356

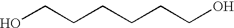

X-357

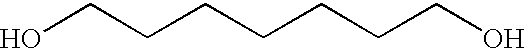

X-358

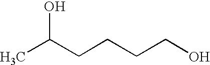

X-359

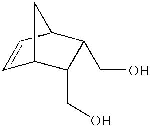

X-360

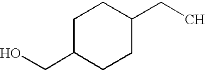

X-361

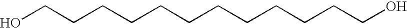

X-362

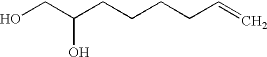

X-363

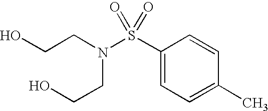

X-364

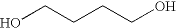

X-365

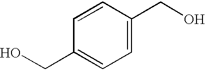

X-366

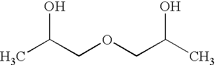

X-367

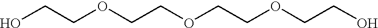

X-368

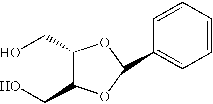

X-369

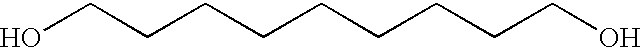

X-370

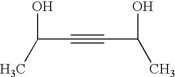

X-371

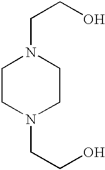

X-372

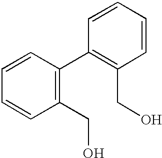

X-373

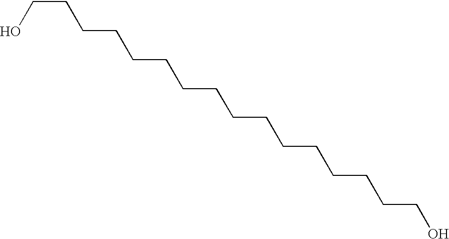

X-374

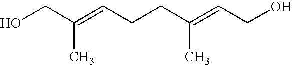

X-375

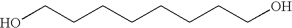

X-376

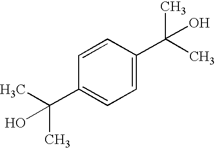

X-377

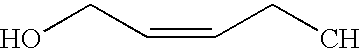

X-378

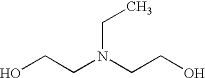

X-379

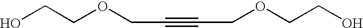

X-380

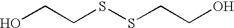

X-381

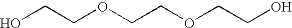

X-382

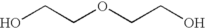

X-383

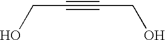

X-384

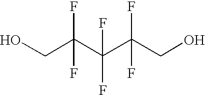

X-385
Dithiols

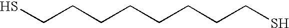

X-386

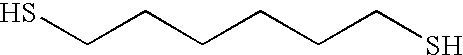

X-387

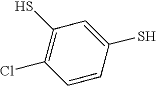

X-388

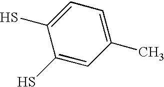

X-389

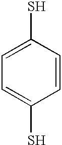

X-390

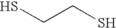

X-391

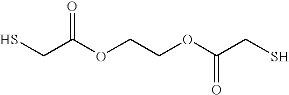

X-392

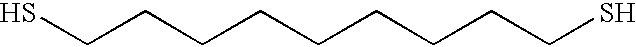

X-393

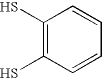

X-394

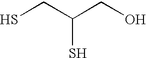

X-395

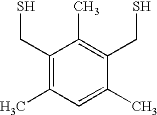

X-396

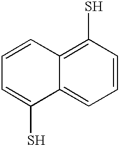

X-397

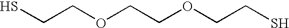

X-398

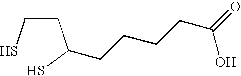

X-399

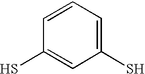

X-400

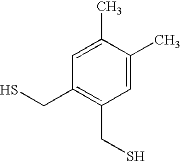

X-401

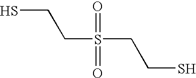

X-402

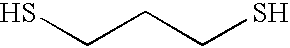

X-403

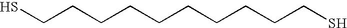

X-404

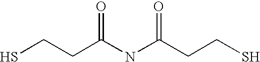

X-405

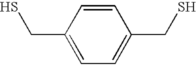

X-406

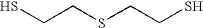

X-407

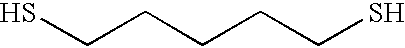

X-408

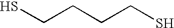

X-409

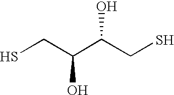

X-410

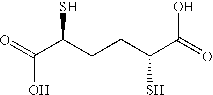

X-411

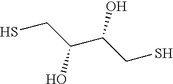

X-412

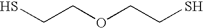

X-413

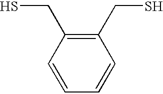

X-414

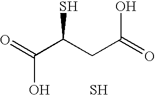

X-415

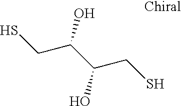

X-416

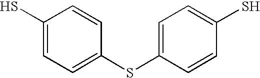

X-417

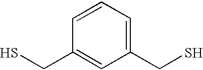

X-418

Representative ligands for use in this invention include, by way of example, L-1 through L-6 as identified below.

| | | | |
|---|---|---|---|
| L-1 | Compound A | L-2 | Compound B |
| L-3 | Compound C | L-4 | Compound D |
| L-5 | Compound B | L-6 | Compound F |

Combinations of ligands (L) and linkers (X) per this invention include, by way example only, homo- and hetero-dimers wherein a first ligand is selected from L-1 through L-6 above and the second ligand and linker is selected from the following:

| | | | | | |
|---|---|---|---|---|---|
| L-1/X-1- | L-1/X-2- | L-1/X-3- | L-1/X-4- | L-1/X-5- | L-1/X-6- |
| L-1/X-7- | L-1/X-8- | L-1/X-9- | L-1/X-10- | L-1/X-11- | L-1/X-12- |
| L-1/X-13- | L-1/X-14- | L-1/X-15- | L-1/X-16- | L-1/X-17- | L-1/X-18- |
| L-1/X-19- | L-1/X-20- | L-1/X-21- | L-1/X-22- | L-1/X-23- | L-1/X-24- |
| L-1/X-25- | L-1/X-26- | L-1/X-27- | L-1/X-28- | L-1/X-29- | L-1/X-30- |
| L-1/X-31- | L-1/X-32- | L-1/X-33- | L-1/X-34- | L-1/X-35- | L-1/X-36- |
| L-1/X-37- | L-1/X-38- | L-1/X-39- | L-1/X-40- | L-1/X-41- | L-1/X-42- |
| L-1/X-43- | L-1/X-44- | L-1/X-45- | L-1/X-46- | L-1/X-47- | L-1/X-48- |
| L-1/X-49- | L-1/X-50- | L-1/X-51- | L-1/X-52- | L-1/X-53- | L-1/X-54- |
| L-1/X-55- | L-1/X-56- | L-1/X-57- | L-1/X-58- | L-1/X-59- | L-1/X-60- |
| L-1/X-61- | L-1/X-62- | L-1/X-63- | L-1/X-64- | L-1/X-65- | L-1/X-66- |
| L-1/X-67- | L-1/X-68- | L-1/X-69- | L-1/X-70- | L-1/X-71- | L-1/X-72- |
| L-1/X-73- | L-1/X-74- | L-1/X-75- | L-1/X-76- | L-1/X-77- | L-1/X-78- |
| L-1/X-79- | L-1/X-80- | L-1/X-81- | L-1/X-82- | L-1/X-83- | L-1/X-84- |
| L-1/X-85- | L-1/X-86- | L-1/X-87- | L-1/X-88- | L-1/X-89- | L-1/X-90- |
| L-1/X-91- | L-1/X-92- | L-1/X-93- | L-1/X-94- | L-1/X-95- | L-1/X-96- |
| L-1/X-97- | L-1/X-98- | L-1/X-99- | L-1/X-100- | L-1/X-101- | L-1/X-102- |
| L-1/X-103- | L-1/X-104- | L-1/X-105- | L-1/X-106- | L-1/X-107- | L-1/X-108- |
| L-1/X-109- | L-l/X-110- | L-1/X-111- | L-1/X-112- | L-1/X-113- | L-1/X-114- |
| L-1/X-115- | L-1/X-116- | L-1/X-117- | L-1/X-118- | L-1/X-119- | L-1/X-120- |
| L-1/X-121- | L-1/X-122- | L-1/X-123- | L-1/X-124- | L-1/X-125- | L-1/X-126- |
| L-1/X-127- | L-1/X-128- | L-1/X-129- | L-1/X-130- | L-1/X-131- | L-1/X-132- |
| L-1/X-133- | L-1/X-134- | L-1/X-135- | L-1/X-136- | L-1/X-137- | L-1/X-138- |
| L-1/X-139- | L-1/X-140- | L-1/X-141- | L-1/X-142- | L-1/X-143- | L-1/X-144- |
| L-1/X-145- | L-1/X-146- | L-1/X-147- | L-1/X-148- | L-1/X-149- | L-1/X-150- |
| L-1/X-151- | L-1/X-152- | L-1/X-153- | L-1/X-154- | L-1/X-155- | L-1/X-156- |
| L-1/X-157- | L-1/X-158- | L-1/X-159- | L-1/X-160- | L-1/X-161- | L-1/X-162- |
| L-1/X-163- | L-1/X-164- | L-1/X-165- | L-1/X-166- | L-1/X-167- | L-1/X-168- |
| L-1/X-169- | L-1/X-170- | L-1/X-171- | L-1/X-172- | | |
| L-1/X-173- | L-1/X-174- | L-1/X-175- | L-1/X-176- | L-1/X-177- | L-1/X-178- |
| L-1/X-179- | L-1/X-180- | L-1/X-181- | L-1/X-182- | L-1/X-183- | L-1/X-184- |
| L-1/X-185- | L-1/X-186- | L-1/X-187- | L-1/X-188- | L-1/X-189- | L-1/X-190- |
| L-1/X-191- | L-1/X-192- | L-1/X-193- | L-1/X-194- | L-1/X-195- | L-1/X-196- |
| L-1/X-197- | L-1/X-198- | L-1/X-199- | L-1/X-200- | L-1/X-201- | L-1/X-202- |
| L-1/X-203- | L-1/X-204- | L-1/X-205- | L-1/X-206- | L-1/X-207- | L-1/X-208- |
| L-1/X-209- | L-1/X-210- | L-1/X-211- | L-1/X-212- | L-1/X-213- | L-1/X-214- |
| L-1/X-215- | L-1/X-216- | L-1/X-217- | L-1/X-218- | L-1/X-219- | L-1/X-220- |
| L-1/X-221- | L-1/X-222- | L-1/X-223- | L-1/X-224- | L-1/X-225- | L-1/X-226- |
| L-1/X-227- | L-1/X-228- | L-1/X-229- | L-1/X-230- | L-1/X-231- | L-1/X-232- |
| L-1/X-233- | L-1/X-234- | L-1/X-235- | L-1/X-236- | L-1/X-237- | L-1/X-238- |
| L-1/X-239- | L-1/X-240- | L-1/X-241- | L-1/X-242- | L-1/X-243- | L-1/X-244- |
| L-1/X-245- | L-1/X-246- | L-1/X-247- | L-1/X-248- | L-1/X-249- | L-1/X-250- |
| L-1/X-251- | L-1/X-252- | L-1/X-253- | L-1/X-254- | L-1/X-255- | L-1/X-256- |
| L-1/X-257- | L-1/X-258- | L-1/X-259- | L-1/X-260- | L-1/X-261- | L-1/X-262- |
| L-1/X-263- | L-1/X-264- | L-1/X-265- | L-1/X-266- | L-1/X-267- | L-1/X-268- |
| L-1/X-269- | L-1/X-270- | L-1/X-271- | L-1/X-272- | L-1/X-273- | L-1/X-274- |
| L-1/X-275- | L-1/X-276- | L-1/X-277- | L-1/X-278- | L-1/X-279- | L-1/X-280- |
| L-1/X-281- | L-1/X-282- | L-1/X-283- | L-1/X-284- | L-1/X-285- | L-1/X-286- |
| L-1/X-287- | L-1/X-288- | L-1/X-289- | L-1/X-290- | L-1/X-291- | L-1/X-292- |
| L-1/X-293- | L-1/X-294- | L-1/X-295- | L-1/X-296- | L-1/X-297- | L-1/X-298- |
| L-1/X-299- | L-1/X-300- | L-1/X-301- | L-1/X-302- | L-1/X-303- | L-1/X-304- |
| L-1/X-305- | L-1/X-306- | L-1/X-307- | L-1/X-308- | L-1/X-309- | L-1/X-310- |
| L-1/X-311- | L-1/X-312- | L-1/X-313- | L-1/X-314- | L-1/X-315- | L-1/X-316- |
| L-1/X-317- | L-1/X-318- | L-1/X-319- | L-1/X-320- | L-1/X-321- | L-1/X-322- |
| L-1/X-323- | L-1/X-324- | L-1/X-325- | L-1/X-326- | L-1/X-327- | L-1/X-328- |
| L-1/X-329- | L-1/X-330- | L-1/X-331- | L-1/X-332- | L-1/X-333- | L-1/X-334- |
| L-1/X-335- | L-1/X-336- | L-1/X-337- | L-1/X-338- | L-1/X-339- | L-1/X-340- |
| L-1/X-341- | L-1/X-342- | L-1/X-343- | L-1/X-344- | L-1/X-345- | L-1/X-346- |
| L-1/X-347- | L-1/X-348- | L-1/X-349- | L-1/X-350- | L-1/X-351- | L-1/X-352- |
| L-1/X-353- | L-1/X-354- | L-1/X-355- | L-l/X-356- | L-1/X-357- | L-1/X-358- |
| L-1/X-359- | L-1/X-360- | L-1/X-361- | L-1/X-362- | L-1/X-363- | L-1/X-364- |
| L-1/X-365- | L-1/X-366- | L-1/X-367- | L-1/X-368- | L-1/X-369- | L-1/X-370- |
| L-1/X-371- | L-1/X-372- | L-1/X-373- | L-1/X-374- | L-1/X-375- | L-1/X-376- |
| L-1/X-377- | L-1/X-378- | L-1/X-379- | L-1/X-380- | L-1/X-381- | L-1/X-382- |
| L-1/X-383- | L-1/X-384- | L-1/X-385- | L-1/X-386- | L-1/X-387- | L-1/X-388- |
| L-1/X-389- | L-1/X-390- | L-1/X-391- | L-1/X-392- | L-1/X-393- | L-1/X-394- |
| L-1/X-395- | L-1/X-396- | L-1/X-397- | L-1/X-398- | L-1/X-399- | L-1/X-400- |
| L-1/X-401- | L-1/X-402- | L-1/X-403- | L-1/X-404- | L-1/X-405- | L-1/X-406- |
| L-1/X-407- | L-1/X-408- | L-1/X-409- | L-1/X-410- | L-1/X-411- | L-1/X-412- |
| L-1/X-413- | L-1/X-414- | L-1/X-415- | L-1/X-416- | L-1/X-417- | L-1/X-418- |
| L-2/X-1- | L-2/X-2- | L-2/X-3- | L-2/X-4- | L-2/X-5- | L-2/X-6- |
| L-2/X-7- | L-2/X-8- | L-2/X-9- | L-2/X-10- | L-2/X-11- | L-2/X-12- |
| L-2/X-13- | L-2/X-14- | L-2/X-15- | L-2/X-16- | L-2/X-17- | L-2/X-18- |
| L-2/X-19- | L-2/X-20- | L-2/X-21- | L-2/X-22- | L-2/X-23- | L-2/X-24- |
| L-2/X-25- | L-2/X-26- | L-2/X-27- | L-2/X-28- | L-2/X-29- | L-2/X-30- |
| L-2/X-31- | L-2/X-32- | L-2/X-33- | L-2/X-34- | L-2/X-35- | L-2/X-36- |
| L-2/X-37- | L-2/X-38- | L-2/X-39- | L-2/X-40- | L-2/X-41- | L-2/X-42- |
| L-2/X-43- | L-2/X-44- | L-2/X-45- | L-2/X-46- | L-2/X-47- | L-2/X-48- |
| L-2/X-49- | L-2/X-50- | L-2/X-51- | L-2/X-52- | L-2/X-53- | L-2/X-54- |
| L-2/X-55- | L-2/X-56- | L-2/X-57- | L-2/X-58- | L-2/X-59- | L-2/X-60- |
| L-2/X-61- | L-2/X-62- | L-2/X-63- | L-2/X-64- | L-2/X-65- | L-2/X-66- |
| L-2/X-67- | L-2/X-68- | L-2/X-69- | L-2/X-70- | L-2/X-71- | L-2/X-72- |
| L-2/X-73- | L-2/X-74- | L-2/X-75- | L-2/X-76- | L-2/X-77- | L-2/X-78- |
| L-2/X-79- | L-2/X-80- | L-2/X-81- | L-2/X-82- | L-2/X-83- | L-2/X-84- |
| L-2/X-85- | L-2/X-86- | L-2/X-87- | L-2/X-88- | L-2/X-89- | L-2/X-90- |
| L-2/X-91- | L-2/X-92- | L-2/X-93- | L-2/X-94- | L-2/X-95- | L-2/X-96- |
| L-2/X-97- | L-2/X-98- | L-2/X-99- | L-2/X-100- | L-2/X-101- | L-2/X-102- |
| L-2/X-103- | L-2/X-104- | L-2/X-105- | L-2/X-106- | L-2/X-107- | L-2/X-108- |
| L-2/X-109- | L-2/X-110- | L-2/X-111- | L-2/X-112- | L-2/X-113- | L-2/X-114- |
| L-2/X-115- | L-2/X-116- | L-2/X-117- | L-2/X-118- | L-2/X-119- | L-2/X-120- |
| L-2/X-121- | L-2/X-122- | L-2/X-123- | L-2/X-124- | L-2/X-125- | L-2/X-126- |
| L-2/X-127- | L-2/X-128- | L-2/X-129- | L-2/X-130- | L-2/X-131- | L-2/X-132- |
| L-2/X-133- | L-2/X-134- | L-2/X-135- | L-2/X-136- | L-2/X-137- | L-2/X-138- |
| L-2/X-139- | L-2/X-140- | L-2/X-141- | L-2/X-142- | L-2/X-143- | L-2/X-144- |
| L-2/X-145- | L-2/X-146- | L-2/X-147- | L-2/X-148- | L-2/X-149- | L-2/X-150- |
| L-2/X-151- | L-2/X-152- | L-2/X-153- | L-2/X-154- | L-2/X-155- | L-2/X-156- |
| L-2/X-157- | L-2/X-158- | L-2/X-159- | L-2/X-160- | L-2/X-161- | L-2/X-162- |
| L-2/X-163- | L-2/X-164- | L-2/X-165- | L-2/X-166- | L-2/X-167- | L-2/X-168- |
| L-2/X-169- | L-2/X-170- | L-2/X-171- | L-2/X-172- | | |
| L-2/X-173- | L-2/X-174- | L-2/X-175- | L-2/X-176- | L-2/X-177- | L-2/X-178- |
| L-2/X-179- | L-2/X-180- | L-2/X-181- | L-2/X-182- | L-2/X-183- | L-2/X-184- |
| L-2/X-185- | L-2/X-186- | L-2/X-187- | L-2/X-188- | L-2/X-189- | L-2/X-190- |
| L-2/X-191- | L-2/X-192- | L-2/X-193- | L-2/X-194- | L-2/X-195- | L-2/X-196- |
| L-2/X-197- | L-2/X-198- | L-2/X-199- | L-2/X-200- | L-2/X-201- | L-2/X-202- |
| L-2/X-203- | L-2/X-204- | L-2/X-205- | L-2/X-206- | L-2/X-207- | L-2/X-208- |
| L-2/X-209- | L-2/X-210- | L-2/X-211- | L-2/X-212- | L-2/X-213- | L-2/X-214- |
| L-2/X-215- | L-2/X-216- | L-2/X-217- | L-2/X-218- | L-2/X-219- | L-2/X-220- |
| L-2/X-221- | L-2/X-222- | L-2/X-223- | L-2/X-224- | L-2/X-225- | L-2/X-226- |
| L-2/X-227- | L-2/X-228- | L-2/X-229- | L-2/X-230- | L-2/X-231- | L-2/X-232- |
| L-2/X-233- | L-2/X-234- | L-2/X-235- | L-2/X-236- | L-2/X-237- | L-2/X-238- |
| L-2/X-239- | L-2/X-240- | L-2/X-241- | L-2/X-242- | L-2/X-243- | L-2/X-244- |
| L-2/X-245- | L-2/X-246- | L-2/X-247- | L-2/X-248- | L-2/X-249- | L-2/X-250- |
| L-2/X-251- | L-2/X-252- | L-2/X-253- | L-2/X-254- | L-2/X-255- | L-2/X-256- |
| L-2/X-257- | L-2/X-258- | L-2/X-259- | L-2/X-260- | L-2/X-261- | L-2/X-262- |
| L-2/X-263- | L-2/X-264- | L-2/X-265- | L-2/X-266- | L-2/X-267- | L-2/X-268- |
| L-2/X-269- | L-2/X-270- | L-2/X-271- | L-2/X-272- | L-2/X-273- | L-2/X-274- |
| L-2/X-275- | L-2/X-276- | L-2/X-277- | L-2/X-278- | L-2/X-279- | L-2/X-280- |
| L-2/X-281- | L-2/X-282- | L-2/X-283- | L-2/X-284- | L-2/X-285- | L-2/X-286- |
| L-2/X-287- | L-2/X-288- | L-2/X-289- | L-2/X-290- | L-2/X-291- | L-2/X-292- |
| L-2/X-293- | L-2/X-294- | L-2/X-295- | L-2/X-296- | L-2/X-297- | L-2/X-298- |
| L-2/X-299- | L-2/X-300- | L-2/X-301- | L-2/X-302- | L-2/X-303- | L-2/X-304- |
| L-2/X-305- | L-2/X-306- | L-2/X-307- | L-2/X-308- | L-2/X-309- | L-2/X-310- |
| L-2/X-311- | L-2/X-312- | L-2/X-313- | L-2/X-314- | L-2/X-315- | L-2/X-316- |
| L-2/X-317- | L-2/X-318- | L-2/X-319- | L-2/X-320- | L-2/X-321- | L-2/X-322- |
| L-2/X-323- | L-2/X-324- | L-2/X-325- | L-2/X-326- | L-2/X-327- | L-2/X-328- |
| L-2/X-329- | L-2/X-330- | L-2/X-331- | L-2/X-332- | L-2/X-333- | L-2/X-334- |
| L-2/X-335- | L-2/X-336- | L-2/X-337- | L-2/X-338- | L-2/X-339- | L-2/X-340- |
| L-2/X-341- | L-2/X-342- | L-2/X-343- | L-2/X-344- | L-2/X-345- | L-2/X-346- |
| L-2/X-347- | L-2/X-348- | L-2/X-349- | L-2/X-350- | L-2/X-351- | L-2/X-352- |
| L-2/X-353- | L-2/X-354- | L-2/X-355- | L-2/X-356- | L-2/X-357- | L-2/X-358- |
| L-2/X-359- | L-2/X-360- | L-2/X-361- | L-2/X-362- | L-2/X-363- | L-2/X-364- |
| L-2/X-365- | L-2/X-366- | L-2/X-367- | L-2/X-368- | L-2/X-369- | L-2/X-370- |
| L-2/X-371- | L-2/X-372- | L-2/X-373- | L-2/X-374- | L-2/X-375- | L-2/X-376- |
| L-2/X-377- | L-2/X-378- | L-2/X-379- | L-2/X-380- | L-2/X-381- | L-2/X-382- |
| L-2/X-383- | L-2/X-384- | L-2/X-385- | L-2/X-386- | L-2/X-387- | L-2/X-388- |
| L-2/X-389- | L-2/X-390- | L-2/X-391- | L-2/X-392- | L-2/X-393- | L-2/X-394- |
| L-2/X-395- | L-2/X-396- | L-2/X-397- | L-2/X-398- | L-2/X-399- | L-2/X-400- |
| L-2/X-401- | L-2/X-402- | L-2/X-403- | L-2/X-404- | L-2/X-405- | L-2/X-406- |
| L-2/X-407- | L-2/X-408- | L-2/X-409- | L-2/X-410- | L-2/X-411- | L-2/X-412- |
| L-2/X-413- | L-2/X-414- | L-2/X-415- | L-2/X-416- | L-2/X-417- | L-2/X-418- |
| L-3/X-1- | L-3/X-2- | L-3/X-3- | L-3/X-4- | L-3/X-5- | L-3/X-6- |
| L-3/X-7- | L-3/X-8- | L-3/X-9- | L-3/X-10- | L-3/X-11- | L-3/X-12- |
| L-3/X-13- | L-3/X-14- | L-3/X-15- | L-3/X-16- | L-3/X-17- | L-3/X-18- |
| L-3/X-19- | L-3/X-20- | L-3/X-21- | L-3/X-22- | L-3/X-23- | L-3/X-24- |
| L-3/X-25- | L-3/X-26- | L-3/X-27- | L-3/X-28- | L-3/X-29- | L-3/X-30- |
| L-3/X-31- | L-3/X-32- | L-3/X-33- | L-3/X-34- | L-3/X-35- | L-3/X-36- |
| L-3/X-37- | L-3/X-38- | L-3/X-39- | L-3/X-40- | L-3/X-41- | L-3/X-42- |
| L-3/X-43- | L-3/X-44- | L-3/X-45- | L-3/X-46- | L-3/X-47- | L-3/X-48- |
| L-3/X-49- | L-3/X-50- | L-3/X-51- | L-3/X-52- | L-3/X-53- | L-3/X-54- |
| L-3/X-55- | L-3/X-56- | L-3/X-57- | L-3/X-58- | L-3/X-59- | L-3/X-60- |
| L-3/X-61- | L-3/X-62- | L-3/X-63- | L-3/X-64- | L-3/X-65- | L-3/X-66- |
| L-3/X-67- | L-3/X-68- | L-3/X-69- | L-3/X-70- | L-3/X-71- | L-3/X-72- |
| L-3/X-73- | L-3/X-74- | L-3/X-75- | L-3/X-76- | L-3/X-77- | L-3/X-78- |
| L-3/X-79- | L-3/X-80- | L-3/X-81- | L-3/X-82- | L-3/X-83- | L-3/X-84- |
| L-3/X-85- | L-3/X-86- | L-3/X-87- | L-3/X-88- | L-3/X-89- | L-3/X-90- |
| L-3/X-91- | L-3/X-92- | L-3/X-93- | L-3/X-94- | L-3/X-95- | L-3/X-96- |
| L-3/X-97- | L-3/X-98- | L-3/X-99- | L-3/X-100- | L-3/X-101- | L-3/X-102- |
| L-3/X-103- | L-3/X-104- | L-3/X-105- | L-3/X-106- | L-3/X-107- | L-3/X-108- |
| L-3/X-109- | L-3/X-110- | L-3/X-111- | L-3/X-112- | L-3/X-113- | L-3/X-114- |
| L-3/X-115- | L-3/X-116- | L-3/X-117- | L-3/X-118- | L-3/X-119- | L-3/X-120- |
| L-3/X-121- | L-3/X-122- | L-3/X-123- | L-3/X-124- | L-3/X-125- | L-3/X-126- |
| L-3/X-127- | L-3/X-128- | L-3/X-129- | L-3/X-130- | L-3/X-131- | L-3/X-132- |
| L-3/X-133- | L-3/X-134- | L-3/X-135- | L-3/X-136- | L-3/X-137- | L-3/X-138- |
| L-3/X-139- | L-3/X-140- | L-3/X-141- | L-3/X-142- | L-3/X-143- | L-3/X-144- |
| L-3/X-145- | L-3/X-146- | L-3/X-147- | L-3/X-148- | L-3/X-149- | L-3/X-150- |
| L-3/X-151- | L-3/X-152- | L-3/X-153- | L-3/X-154- | L-3/X-155- | L-3/X-156- |
| L-3/X-157- | L-3/X-158- | L-3/X-159- | L-3/X-160- | L-3/X-161- | L-3/X-162- |
| L-3/X-163- | L-3/X-164- | L-3/X-165- | L-3/X-166- | L-3/X-167- | L-3/X-168- |
| L-3/X-169- | L-3/X-170- | L-3/X-171- | L-3/X-172- | | |
| L-3/X-173- | L-3/X-174- | L-3/X-175- | L-3/X-176- | L-3/X-177- | L-3/X-178- |
| L-3/X-179- | L-3/X-180- | L-3/X-181- | L-3/X-182- | L-3/X-183- | L-3/X-184- |
| L-3/X-185- | L-3/X-186- | L-3/X-187- | L-3/X-188- | L-3/X-189- | L-3/X-190- |
| L-3/X-191- | L-3/X-192- | L-3/X-193- | L-3/X-194- | L-3/X-195- | L-3/X-196- |
| L-3/X-197- | L-3/X-198- | L-3/X-199- | L-3/X-200- | L-3/X-201- | L-3/X-202- |
| L-3/X-203- | L-3/X-204- | L-3/X-205- | L-3/X-206- | L-3/X-207- | L-3/X-208- |
| L-3/X-209- | L-3/X-210- | L-3/X-211- | L-3/X-212- | L-3/X-213- | L-3/X-214- |
| L-3/X-215- | L-3/X-216- | L-3/X-217- | L-3/X-218- | L-3/X-219- | L-3/X-220- |
| L-3/X-221- | L-3/X-222- | L-3/X-223- | L-3/X-224- | L-3/X-225- | L-3/X-226- |
| L-3/X-227- | L-3/X-228- | L-3/X-229- | L-3/X-230- | L-3/X-231- | L-3/X-232- |
| L-3/X-233- | L-3/X-234- | L-3/X-235- | L-3/X-236- | L-3/X-237- | L-3/X-238- |
| L-3/X-239- | L-3/X-240- | L-3/X-241- | L-3/X-242- | L-3/X-243- | L-3/X-244- |
| L-3/X-245- | L-3/X-246- | L-3/X-247- | L-3/X-248- | L-3/X-249- | L-3/X-250- |
| L-3/X-251- | L-3/X-252- | L-3/X-253- | L-3/X-254- | L-3/X-255- | L-3/X-256- |
| L-3/X-257- | L-3/X-258- | L-3/X-259- | L-3/X-260- | L-3/X-261- | L-3/X-262- |
| L-3/X-263- | L-3/X-264- | L-3/X-265- | L-3/X-266- | L-3/X-267- | L-3/X-268- |
| L-3/X-269- | L-3/X-270- | L-3/X-271- | L-3/X-272- | L-3/X-273- | L-3/X-274- |
| L-3/X-275- | L-3/X-276- | L-3/X-277- | L-3/X-278- | L-3/X-279- | L-3/X-280- |
| L-3/X-281- | L-3/X-282- | L-3/X-283- | L-3/X-284- | L-3/X-285- | L-3/X-286- |
| L-3/X-287- | L-3/X-288- | L-3/X-289- | L-3/X-290- | L-3/X-291- | L-3/X-292- |
| L-3/X-293- | L-3/X-294- | L-3/X-295- | L-3/X-296- | L-3/X-297- | L-3/X-298- |
| L-3/X-299- | L-3/X-300- | L-3/X-301- | L-3/X-302- | L-3/X-303- | L-3/X-304- |
| L-3/X-305- | L-3/X-306- | L-3/X-307- | L-3/X-308- | L-3/X-309- | L-3/X-310- |
| L-3/X-311- | L-3/X-312- | L-3/X-313- | L-3/X-314- | L-3/X-315- | L-3/X-316- |
| L-3/X-317- | L-3/X-318- | L-3/X-319- | L-3/X-320- | L-3/X-321- | L-3/X-322- |
| L-3/X-323- | L-3/X-324- | L-3/X-325- | L-3/X-326- | L-3/X-327- | L-3/X-328- |
| L-3/X-329- | L-3/X-330- | L-3/X-331- | L-3/X-332- | L-3/X-333- | L-3/X-334- |
| L-3/X-335- | L-3/X-336- | L-3/X-337- | L-3/X-338- | L-3/X-339- | L-3/X-340- |
| L-3/X-341- | L-3/X-342- | L-3/X-343- | L-3/X-344- | L-3/X-345- | L-3/X-346- |
| L-3/X-347- | L-3/X-348- | L-3/X-349- | L-3/X-350- | L-3/X-351- | L-3/X-352- |
| L-3/X-353- | L-3/X-354- | L-3/X-355- | L-3/X-356- | L-3/X-357- | L-3/X-358- |
| L-3/X-359- | L-3/X-360- | L-3/X-361- | L-3/X-362- | L-3/X-363- | L-3/X-364- |
| L-3/X-365- | L-3/X-366- | L-3/X-367- | L-3/X-368- | L-3/X-369- | L-3/X-370- |
| L-3/X-371- | L-3/X-372- | L-3/X-373- | L-3/X-374- | L-3/X-375- | L-3/X-376- |
| L-3/X-377- | L-3/X-378- | L-3/X-379- | L-3/X-380- | L-3/X-381- | L-3/X-382- |
| L-3/X-383- | L-3/X-384- | L-3/X-385- | L-3/X-386- | L-3/X-387- | L-3/X-388- |
| L-3/X-389- | L-3/X-390- | L-3/X-391- | L-3/X-392- | L-3/X-393- | L-3/X-394- |
| L-3/X-395- | L-3/X-396- | L-3/X-397- | L-3/X-398- | L-3/X-399- | L-3/X-400- |
| L-3/X-401- | L-3/X-402- | L-3/X-403- | L-3/X-404- | L-3/X-405- | L-3/X-406- |
| L-3/X-407- | L-3/X-408- | L-3/X-409- | L-3/X-410- | L-3/X-411- | L-3/X-412- |
| L-3/X-413- | L-3/X-414- | L-3/X-415- | L-3/X-416- | L-3/X-417- | L-3/X-418- |
| L-4/X-1- | L-4/X-2- | L-4/X-3- | L-4/X-4- | L-4/X-5- | L-4/X-6- |
| L-4/X-7- | L-4/X-8- | L-4/X-9- | L-4/X-10- | L-4/X-11- | L-4/X-12- |
| L-4/X-13- | L-4/X-14- | L-4/X-15- | L-4/X-16- | L-4/X-17- | L-4/X-18- |
| L-4/X-19- | L-4/X-20- | L-4/X-21- | L-4/X-22- | L-4/X-23- | L-4/X-24- |
| L-4/X-25- | L-4/X-26- | L-4/X-27- | L-4/X-28- | L-4/X-29- | L-4/X-30- |
| L-4/X-31- | L-4/X-32- | L-4/X-33- | L-4/X-34- | L-4/X-35- | L-4/X-36- |
| L-4/X-37- | L-4/X-38- | L-4/X-39- | L-4/X-40- | L-4/X-41- | L-4/X-42- |
| L-4/X-43- | L-4/X-44- | L-4/X-45- | L-4/X-46- | L-4/X-47- | L-4/X-48- |
| L-4/X-49- | L-4/X-50- | L-4/X-51- | L-4/X-52- | L-4/X-53- | L-4/X-54- |
| L-4/X-55- | L-4/X-56- | L-4/X-57- | L-4/X-58- | L-4/X-59- | L-4/X-60- |
| L-4/X-61- | L-4/X-62- | L-4/X-63- | L-4/X-64- | L-4/X-65- | L-4/X-66- |
| L-4/X-67- | L-4/X-68- | L-4/X-69- | L-4/X-70- | L-4/X-71- | L-4/X-72- |
| L-4/X-73- | L-4/X-74- | L-4/X-75- | L-4/X-76- | L-4/X-77- | L-4/X-78- |
| L-4/X-79- | L-4/X-80- | L-4/X-81- | L-4/X-82- | L-4/X-83- | L-4/X-84- |
| L-4/X-85- | L-4/X-86- | L-4/X-87- | L-4/X-88- | L-4/X-89- | L-4/X-90- |
| L-4/X-91- | L-4/X-92- | L-4/X-93- | L-4/X-94- | L-4/X-95- | L-4/X-96- |
| L-4/X-97- | L-4/X-98- | L-4/X-99- | L-4/X-100- | L-4/X-101- | L-4/X-102- |
| L-4/X-103- | L-4/X-104- | L-4/X-105- | L-4/X-106- | L-4/X-107- | L-4/X-108- |
| L-4/X-109- | L-4/X-110- | L-4/X-111- | L-4/X-112- | L-4/X-113- | L-4/X-114- |
| L-4/X-115- | L-4/X-116- | L-4/X-117- | L-4/X-118- | L-4/X-119- | L-4/X-120- |
| L-4/X-121- | L-4/X-122- | L-4/X-123- | L-4/X-124- | L-4/X-125- | L-4/X-126- |
| L-4/X-127- | L-4/X-128- | L-4/X-129- | L-4/X-130- | L-4/X-131- | L-4/X-132- |
| L-4/X-133- | L-4/X-134- | L-4/X-135- | L-4/X-136- | L-4/X-137- | L-4/X-138- |
| L-4/X-139- | L-4/X-140- | L-4/X-141- | L-4/X-142- | L-4/X-143- | L-4/X-144- |
| L-4/X-145- | L-4/X-146- | L-4/X-147- | L-4/X-148- | L-4/X-149- | L-4/X-150- |
| L-4/X-151- | L-4/X-152- | L-4/X-153- | L-4/X-154- | L-4/X-155- | L-4/X-156- |
| L-4/X-157- | L-4/X-158- | L-4/X-159- | L-4/X-160- | L-4/X-161- | L-4/X-162- |
| L-4/X-163- | L-4/X-164- | L-4/X-165- | L-4/X-166- | L-4/X-167- | L-4/X-168- |
| L-4/X-169- | L-4/X-170- | L-4/X-171- | L-4/X-172- | | |
| L-4/X-173- | L-4/X-174- | L-4/X-175- | L-4/X-176- | L-4/X-177- | L-4/X-178- |
| L-4/X-179- | L-4/X-180- | L-4/X-181- | L-4/X-182- | L-4/X-183- | L-4/X-184- |
| L-4/X-185- | L-4/X-186- | L-4/X-187- | L-4/X-188- | L-4/X-189- | L-4/X-190- |
| L-4/X-191- | L-4/X-192- | L-4/X-193- | L-4/X-194- | L-4/X-195- | L-4/X-196- |
| L-4/X-197- | L-4/X-198- | L-4/X-199- | L-4/X-200- | L-4/X-201- | L-4/X-202- |
| L-4/X-203- | L-4/X-204- | L-4/X-205- | L-4/X-206- | L-4/X-207- | L-4/X-208- |
| L-4/X-209- | L-4/X-210- | L-4/X-211- | L-4/X-212- | L-4/X-213- | L-4/X-214- |
| L-4/X-215- | L-4/X-216- | L-4/X-217- | L-4/X-218- | L-4/X-219- | L-4/X-220- |
| L-4/X-221- | L-4/X-222- | L-4/X-223- | L-4/X-224- | L-4/X-225- | L-4/X-226- |
| L-4/X-227- | L-4/X-228- | L-4/X-229- | L-4/X-230- | L-4/X-231- | L-4/X-232- |
| L-4/X-233- | L-4/X-234- | L-4/X-235- | L-4/X-236- | L-4/X-237- | L-4/X-238- |
| L-4/X-239- | L-4/X-240- | L-4/X-241- | L-4/X-242- | L-4/X-243- | L-4/X-244- |
| L-4/X-245- | L-4/X-246- | L-4/X-247- | L-4/X-248- | L-4/X-249- | L-4/X-250- |
| L-4/X-251- | L-4/X-252- | L-4/X-253- | L-4/X-254- | L-4/X-255- | L-4/X-256- |
| L-4/X-257- | L-4/X-258- | L-4/X-259- | L-4/X-260- | L-4/X-261- | L-4/X-262- |
| L-4/X-263- | L-4/X-264- | L-4/X-265- | L-4/X-266- | L-4/X-267- | L-4/X-268- |
| L-4/X-269- | L-4/X-270- | L-4/X-271- | L-4/X-272- | L-4/X-273- | L-4/X-274- |
| L-4/X-275- | L-4/X-276- | L-4/X-277- | L-4/X-278- | L-4/X-279- | L-4/X-280- |
| L-4/X-281- | L-4/X-282- | L-4/X-283- | L-4/X-284- | L-4/X-285- | L-4/X-286- |
| L-4/X-287- | L-4/X-288- | L-4/X-289- | L-4/X-290- | L-4/X-291- | L-4/X-292- |
| L-4/X-293- | L-4/X-294- | L-4/X-295- | L-4/X-296- | L-4/X-297- | L-4/X-298- |
| L-4/X-299- | L-4/X-300- | L-4/X-301- | L-4/X-302- | L-4/X-303- | L-4/X-304- |
| L-4/X-305- | L-4/X-306- | L-4/X-307- | L-4/X-308- | L-4/X-309- | L-4/X-310- |
| L-4/X-311- | L-4/X-312- | L-4/X-313- | L-4/X-314- | L-4/X-315- | L-4/X-316- |
| L-4/X-317- | L-4/X-318- | L-4/X-319- | L-4/X-320- | L-4/X-321- | L-4/X-322- |
| L-4/X-323- | L-4/X-324- | L-4/X-325- | L-4/X-326- | L-4/X-327- | L-4/X-328- |
| L-4/X-329- | L-4/X-330- | L-4/X-331- | L-4/X-332- | L-4/X-333- | L-4/X-334- |
| L-4/X-335- | L-4/X-336- | L-4/X-337- | L-4/X-338- | L-4/X-339- | L-4/X-340- |
| L-4/X-341- | L-4/X-342- | L-4/X-343- | L-4/X-344- | L-4/X-345- | L-4/X-346- |
| L-4/X-347- | L-4/X-348- | L-4/X-349- | L-4/X-350- | L-4/X-351- | L-4/X-352- |
| L-4/X-353- | L-4/X-354- | L-4/X-355- | L-4/X-356- | L-4/X-357- | L-4/X-358- |
| L-4/X-359- | L-4/X-360- | L-4/X-361- | L-4/X-362- | L-4/X-363- | L-4/X-364- |
| L-4/X-365- | L-4/X-366- | L-4/X-367- | L-4/X-368- | L-4/X-369- | L-4/X-370- |
| L-4/X-371- | L-4/X-372- | L-4/X-373- | L-4/X-374- | L-4/X-375- | L-4/X-376- |
| L-4/X-377- | L-4/X-378- | L-4/X-379- | L-4/X-380- | L-4/X-381- | L-4/X-382- |
| L-4/X-383- | L-4/X-384- | L-4/X-385- | L-4/X-386- | L-4/X-387- | L-4/X-388- |
| L-4/X-389- | L-4/X-390- | L-4/X-391- | L-4/X-392- | L-4/X-393- | L-4/X-394- |
| L-4/X-395- | L-4/X-396- | L-4/X-397- | L-4/X-398- | L-4/X-399- | L-4/X-400- |
| L-4/X-401- | L-4/X-402- | L-4/X-403- | L-4/X-404- | L-4/X-405- | L-4/X-406- |
| L-4/X-407- | L-4/X-408- | L-4/X-409- | L-4/X-410- | L-4/X-411- | L-4/X-412- |
| L-4/X-413- | L-4/X-414- | L-4/X-415- | L-4/X-416- | L-4/X-417- | L-4/X-418- |
| L-5/X-1- | L-5/X-2- | L-5/X-3- | L-5/X-4- | L-5/X-5- | L-5/X-6- |
| L-5/X-7- | L-5/X-8- | L-5/X-9- | L-5/X-10- | L-5/X-11- | L-5/X-12- |
| L-5/X-13- | L-5/X-14- | L-5/X-15- | L-5/X-16- | L-5/X-17- | L-5/X-18- |
| L-5/X-19- | L-5/X-20- | L-5/X-21- | L-5/X-22- | L-5/X-23- | L-5/X-24- |
| L-5/X-25- | L-5/X-26- | L-5/X-27- | L-5/X-28- | L-5/X-29- | L-5/X-30- |
| L-5/X-31- | L-5/X-32- | L-5/X-33- | L-5/X-34- | L-5/X-35- | L-5/X-36- |
| L-5/X-37- | L-5/X-38- | L-5/X-39- | L-5/X-40- | L-5/X-41- | L-5/X-42- |
| L-5/X-43- | L-5/X-44- | L-5/X-45- | L-5/X-46- | L-5/X-47- | L-5/X-48- |
| L-5/X-49- | L-5/X-50- | L-5/X-51- | L-5/X-52- | L-5/X-53- | L-5/X-54- |
| L-5/X-55- | L-5/X-56- | L-5/X-57- | L-5/X-58- | L-5/X-59- | L-5/X-60- |
| L-5/X-61- | L-5/X-62- | L-5/X-63- | L-5/X-64- | L-5/X-65- | L-5/X-66- |
| L-5/X-67- | L-5/X-68- | L-5/X-69- | L-5/X-70- | L-5/X-71- | L-5/X-72- |
| L-5/X-73- | L-5/X-74- | L-5/X-75- | L-5/X-76- | L-5/X-77- | L-5/X-78- |
| L-5/X-79- | L-5/X-80- | L-5/X-81- | L-5/X-82- | L-5/X-83- | L-5/X-84- |
| L-5/X-85- | L-5/X-86- | L-5/X-87- | L-5/X-88- | L-5/X-89- | L-5/X-90- |
| L-5/X-91- | L-5/X-92- | L-5/X-93- | L-5/X-94- | L-5/X-95- | L-5/X-96- |
| L-5/X-97- | L-5/X-98- | L-5/X-99- | L-5/X-100- | L-5/X-101- | L-5/X-102- |
| L-5/X-103- | L-5/X-104- | L-5/X-105- | L-5/X-106- | L-5/X-107- | L-5/X-108- |
| L-5/X-109- | L-5/X-110- | L-5/X-111- | L-5/X-112- | L-5/X-113- | L-5/X-114- |
| L-5/X-115- | L-5/X-116- | L-5/X-117- | L-5/X-118- | L-5/X-119- | L-5/X-120- |
| L-5/X-121- | L-5/X-122- | L-5/X-123- | L-5/X-124- | L-5/X-125- | L-5/X-126- |
| L-5/X-127- | L-5/X-128- | L-5/X-129- | L-5/X-130- | L-5/X-131- | L-5/X-132- |
| L-5/X-133- | L-5/X-134- | L-5/X-135- | L-5/X-136- | L-5/X-137- | L-5/X-138- |
| L-5/X-139- | L-5/X-140- | L-5/X-141- | L-5/X-142- | L-5/X-143- | L-5/X-144- |
| L-5/X-145- | L-5/X-146- | L-5/X-147- | L-5/X-148- | L-5/X-149- | L-5/X-150- |
| L-5/X-151- | L-5/X-152- | L-5/X-153- | L-5/X-154- | L-5/X-155- | L-5/X-156- |
| L-5/X-157- | L-5/X-158- | L-5/X-159- | L-5/X-160- | L-5/X-161- | L-5/X-162- |
| L-5/X-163- | L-5/X-164- | L-5/X-165- | L-5/X-166- | L-5/X-167- | L-5/X-168- |
| L-5/X-169- | L-5/X-170- | L-5/X-171- | L-5/X-172- | | |
| L-5/X-173- | L-5/X-174- | L-5/X-175- | L-5/X-176- | L-5/X-177- | L-5/X-178- |
| L-5/X-179- | L-5/X-180- | L-5/X-181- | L-5/X-182- | L-5/X-183- | L-5/X-184- |
| L-5/X-185- | L-5/X-186- | L-5/X-187- | L-5/X-188- | L-5/X-189- | L-5/X-190- |
| L-5/X-191- | L-5/X-192- | L-5/X-193- | L-5/X-194- | L-5/X-195- | L-5/X-196- |
| L-5/X-197- | L-5/X-198- | L-5/X-199- | L-5/X-200- | L-5/X-201- | L-5/X-202- |
| L-5/X-203- | L-5/X-204- | L-5/X-205- | L-5/X-206- | L-5/X-207- | L-5/X-208- |
| L-5/X-209- | L-5/X-210- | L-5/X-211- | L-5/X-212- | L-5/X-213- | L-5/X-214- |
| L-5/X-215- | L-5/X-216- | L-5/X-217- | L-5/X-218- | L-5/X-219- | L-5/X-220- |
| L-5/X-221- | L-5/X-222- | L-5/X-223- | L-5/X-224- | L-5/X-225- | L-5/X-226- |
| L-5/X-227- | L-5/X-228- | L-5/X-229- | L-5/X-230- | L-5/X-231- | L-5/X-232- |
| L-5/X-233- | L-5/X-234- | L-5/X-235- | L-5/X-236- | L-5/X-237- | L-5/X-238- |
| L-5/X-239- | L-5/X-240- | L-5/X-241- | L-5/X-242- | L-5/X-243- | L-5/X-244- |
| L-5/X-245- | L-5/X-246- | L-5/X-247- | L-5/X-248- | L-5/X-249- | L-5/X-250- |
| L-5/X-251- | L-5/X-252- | L-5/X-253- | L-5/X-254- | L-5/X-255- | L-5/X-256- |
| L-5/X-257- | L-5/X-258- | L-5/X-259- | L-5/X-260- | L-5/X-261- | L-5/X-262- |
| L-5/X-263- | L-5/X-264- | L-5/X-265- | L-5/X-266- | L-5/X-267- | L-5/X-268- |
| L-5/X-269- | L-5/X-270- | L-5/X-271- | L-5/X-272- | L-5/X-273- | L-5/X-274- |
| L-5/X-275- | L-5/X-276- | L-5/X-277- | L-5/X-278- | L-5/X-279- | L-5/X-280- |
| L-5/X-281- | L-5/X-282- | L-5/X-283- | L-5/X-284- | L-5/X-285- | L-5/X-286- |
| L-5/X-287- | L-5/X-288- | L-5/X-289- | L-5/X-290- | L-5/X-291- | L-5/X-292- |
| L-5/X-293- | L-5/X-294- | L-5/X-295- | L-5/X-296- | L-5/X-297- | L-5/X-298- |
| L-5/X-299- | L-5/X-300- | L-5/X-301- | L-5/X-302- | L-5/X-303- | L-5/X-304- |
| L-5/X-305- | L-5/X-306- | L-5/X-307- | L-5/X-308- | L-5/X-309- | L-5/X-310- |
| L-5/X-311- | L-5/X-312- | L-5/X-313- | L-5/X-314- | L-5/X-315- | L-5/X-316- |
| L-5/X-317- | L-5/X-318- | L-5/X-319- | L-5/X-320- | L-5/X-321- | L-5/X-322- |
| L-5/X-323- | L-5/X-324- | L-5/X-325- | L-5/X-326- | L-5/X-327- | L-5/X-328- |
| L-5/X-329- | L-5/X-330- | L-5/X-331- | L-5/X-332- | L-5/X-333- | L-5/X-334- |
| L-5/X-335- | L-5/X-336- | L-5/X-337- | L-5/X-338- | L-5/X-339- | L-5/X-340- |
| L-5/X-341- | L-5/X-342- | L-5/X-343- | L-5/X-344- | L-5/X-345- | L-5/X-346- |
| L-5/X-347- | L-5/X-348- | L-5/X-349- | L-5/X-350- | L-5/X-351- | L-5/X-352- |
| L-5/X-353- | L-5/X-354- | L-5/X-355- | L-5/X-356- | L-5/X-357- | L-5/X-358- |
| L-5/X-359- | L-5/X-360- | L-5/X-361- | L-5/X-362- | L-5/X-363- | L-5/X-364- |
| L-5/X-365- | L-5/X-366- | L-5/X-367- | L-5/X-368- | L-5/X-369- | L-5/X-370- |
| L-5/X-371- | L-5/X-372- | L-5/X-373- | L-5/X-374- | L-5/X-375- | L-5/X-376- |
| L-5/X-377- | L-5/X-378- | L-5/X-379- | L-5/X-380- | L-5/X-381- | L-5/X-382- |
| L-5/X-383- | L-5/X-384- | L-5/X-385- | L-5/X-386- | L-5/X-387- | L-5/X-388- |
| L-5/X-389- | L-5/X-390- | L-5/X-391- | L-5/X-392- | L-5/X-393- | L-5/X-394- |
| L-5/X-395- | L-5/X-396- | L-5/X-397- | L-5/X-398- | L-5/X-399- | L-5/X-400- |
| L-5/X-401- | L-5/X-402- | L-5/X-403- | L-5/X-404- | L-5/X-405- | L-5/X-406- |
| L-5/X-407- | L-5/X-408- | L-5/X-409- | L-5/X-410- | L-5/X-411- | L-5/X-412- |
| L-5/X-413- | L-5/X-414- | L-5/X-415- | L-5/X-416- | L-5/X-417- | L-5/X-418- |
| L-6/X-1- | L-6/X-2- | L-6/X-3- | L-6/X-4- | L-6/X-5- | L-6/X-6- |
| L-6/X-7- | L-6/X-8- | L-6/X-9- | L-6/X-10- | L-6/X-11- | L-6/X-12- |
| L-6/X-13- | L-6/X-14- | L-6/X-15- | L-6/X-16- | L-6/X-17- | L-6/X-18- |
| L-6/X-19- | L-6/X-20- | L-6/X-21- | L-6/X-22- | L-6/X-23- | L-6/X-24- |
| L-6/X-25- | L-6/X-26- | L-6/X-27- | L-6/X-28- | L-6/X-29- | L-6/X-30- |
| L-6/X-31- | L-6/X-32- | L-6/X-33- | L-6/X-34- | L-6/X-35- | L-6/X-36- |
| L-6/X-37- | L-6/X-38- | L-6/X-39- | L-6/X-40- | L-6/X-41- | L-6/X-42- |
| L-6/X-43- | L-6/X-44- | L-6/X-45- | L-6/X-46- | L-6/X-47- | L-6/X-48- |
| L-6/X-49- | L-6/X-50- | L-6/X-51- | L-6/X-52- | L-6/X-53- | L-6/X-54- |
| L-6/X-55- | L-6/X-56- | L-6/X-57- | L-6/X-58- | L-6/X-59- | L-6/X-60- |
| L-6/X-61- | L-6/X-62- | L-6/X-63- | L-6/X-64- | L-6/X-65- | L-6/X-66- |
| L-6/X-67- | L-6/X-68- | L-6/X-69- | L-6/X-70- | L-6/X-71- | L-6/X-72- |
| L-6/X-73- | L-6/X-74- | L-6/X-75- | L-6/X-76- | L-6/X-77- | L-6/X-78- |
| L-6/X-79- | L-6/X-80- | L-6/X-81- | L-6/X-82- | L-6/X-83- | L-6/X-84- |
| L-6/X-85- | L-6/X-86- | L-6/X-87- | L-6/X-88- | L-6/X-89- | L-6/X-90- |
| L-6/X-91- | L-6/X-92- | L-6/X-93- | L-6/X-94- | L-6/X-95- | L-6/X-96- |
| L-6/X-97- | L-6/X-98- | L-6/X-99- | L-6/X-100- | L-6/X-101- | L-6/X-102- |
| L-6/X-103- | L-6/X-104- | L-6/X-105- | L-6/X-106- | L-6/X-107- | L-6/X-108- |
| L-6/X-109- | L-6/X-110- | L-6/X-111- | L-6/X-112- | L-6/X-113- | L-6/X-114- |
| L-6/X-115- | L-6/X-116- | L-6/X-117- | L-6/X-118- | L-6/X-119- | L-6/X-120- |
| L-6/X-121- | L-6/X-122- | L-6/X-123- | L-6/X-124- | L-6/X-125- | L-6/X-126- |
| L-6/X-127- | L-6/X-128- | L-6/X-129- | L-6/X-130- | L-6/X-131- | L-6/X-132- |
| L-6/X-133- | L-6/X-134- | L-6/X-135- | L-6/X-136- | L-6/X-137- | L-6/X-138- |
| L-6/X-139- | L-6/X-140- | L-6/X-141- | L-6/X-142- | L-6/X-143- | L-6/X-144- |
| L-6/X-145- | L-6/X-146- | L-6/X-147- | L-6/X-148- | L-6/X-149- | L-6/X-150- |
| L-6/X-151- | L-6/X-152- | L-6/X-153- | L-6/X-154- | L-6/X-155- | L-6/X-156- |
| L-6/X-157- | L-6/X-158- | L-6/X-159- | L-6/X-160- | L-6/X-161- | L-6/X-162- |
| L-6/X-163- | L-6/X-164- | L-6/X-165- | L-6/X-166- | L-6/X-167- | L-6/X-168- |
| L-6/X-169- | L-6/X-170- | L-6/X-171- | L-6/X-172- | | |
| L-6/X-173- | L-6/X-174- | L-6/X-175- | L-6/X-176- | L-6/X-177- | L-6/X-178- |
| L-6/X-179- | L-6/X-180- | L-6/X-181- | L-6/X-182- | L-6/X-183- | L-6/X-184- |
| L-6/X-185- | L-6/X-186- | L-6/X-187- | L-6/X-188- | L-6/X-189- | L-6/X-190- |
| L-6/X-191- | L-6/X-192- | L-6/X-193- | L-6/X-194- | L-6/X-195- | L-6/X-196- |
| L-6/X-197- | L-6/X-198- | L-6/X-199- | L-6/X-200- | L-6/X-201- | L-6/X-202- |
| L-6/X-203- | L-6/X-204- | L-6/X-205- | L-6/X-206- | L-6/X-207- | L-6/X-208- |
| L-6/X-209- | L-6/X-210- | L-6/X-211- | L-6/X-212- | L-6/X-213- | L-6/X-214- |
| L-6/X-215- | L-6/X-216- | L-6/X-217- | L-6/X-218- | L-6/X-219- | L-6/X-220- |
| L-6/X-221- | L-6/X-222- | L-6/X-223- | L-6/X-224- | L-6/X-225- | L-6/X-226- |
| L-6/X-227- | L-6/X-228- | L-6/X-229- | L-6/X-230- | L-6/X-231- | L-6/X-232- |
| L-6/X-233- | L-6/X-234- | L-6/X-235- | L-6/X-236- | L-6/X-237- | L-6/X-238- |
| L-6/X-239- | L-6/X-240- | L-6/X-241- | L-6/X-242- | L-6/X-243- | L-6/X-244- |
| L-6/X-245- | L-6/X-246- | L-6/X-247- | L-6/X-248- | L-6/X-249- | L-6/X-250- |
| L-6/X-251- | L-6/X-252- | L-6/X-253- | L-6/X-254- | L-6/X-255- | L-6/X-256- |
| L-6/X-257- | L-6/X-258- | L-6/X-259- | L-6/X-260- | L-6/X-261- | L-6/X-262- |
| L-6/X-263- | L-6/X-264- | L-6/X-265- | L-6/X-266- | L-6/X-267- | L-6/X-268- |
| L-6/X-269- | L-6/X-270- | L-6/X-271- | L-6/X-272- | L-6/X-273- | L-6/X-274- |
| L-6/X-275- | L-6/X-276- | L-6/X-277- | L-6/X-278- | L-6/X-279- | L-6/X-280- |
| L-6/X-281- | L-6/X-282- | L-6/X-283- | L-6/X-284- | L-6/X-285- | L-6/X-286- |
| L-6/X-287- | L-6/X-288- | L-6/X-289- | L-6/X-290- | L-6/X-291- | L-6/X-292- |
| L-6/X-293- | L-6/X-294- | L-6/X-295- | L-6/X-296- | L-6/X-297- | L-6/X-298- |
| L-6/X-299- | L-6/X-300- | L-6/X-301- | L-6/X-302- | L-6/X-303- | L-6/X-304- |
| L-6/X-305- | L-6/X-306- | L-6/X-307- | L-6/X-308- | L-6/X-309- | L-6/X-310- |
| L-6/X-311- | L-6/X-312- | L-6/X-313- | L-6/X-314- | L-6/X-315- | L-6/X-316- |
| L-6/X-317- | L-6/X-318- | L-6/X-319- | L-6/X-320- | L-6/X-321- | L-6/X-322- |
| L-6/X-323- | L-6/X-324- | L-6/X-325- | L-6/X-326- | L-6/X-327- | L-6/X-328- |
| L-6/X-329- | L-6/X-330- | L-6/X-331- | L-6/X-332- | L-6/X-333- | L-6/X-334- |
| L-6/X-335- | L-6/X-336- | L-6/X-337- | L-6/X-338- | L-6/X-339- | L-6/X-340- |
| L-6/X-341- | L-6/X-342- | L-6/X-343- | L-6/X-344- | L-6/X-345- | L-6/X-346- |
| L-6/X-347- | L-6/X-348- | L-6/X-349- | L-6/X-350- | L-6/X-351- | L-6/X-352- |
| L-6/X-353- | L-6/X-354- | L-6/X-355- | L-6/X-356- | L-6/X-357- | L-6/X-358- |
| L-6/X-359- | L-6/X-360- | L-6/X-361- | L-6/X-362- | L-6/X-363- | L-6/X-364- |
| L-6/X-365- | L-6/X-366- | L-6/X-367- | L-6/X-368- | L-6/X-369- | L-6/X-370- |
| L-6/X-371- | L-6/X-372- | L-6/X-373- | L-6/X-374- | L-6/X-375- | L-6/X-376- |
| L-6/X-377- | L-6/X-378- | L-6/X-379- | L-6/X-380- | L-6/X-381- | L-6/X-382- |
| L-6/X-383- | L-6/X-384- | L-6/X-385- | L-6/X-386- | L-6/X-387- | L-6/X-388- |
| L-6/X-389- | L-6/X-390- | L-6/X-391- | L-6/X-392- | L-6/X-393- | L-6/X-394- |
| L-6/X-395- | L-6/X-396- | L-6/X-397- | L-6/X-398- | L-6/X-399- | L-6/X-400- |
| L-6/X-401- | L-6/X-402- | L-6/X-403- | L-6/X-404- | L-6/X-405- | L-6/X-406- |
| L-6/X-407- | L-6/X-408- | L-6/X-409- | L-6/X-410- | L-6/X-411- | L-6/X-412- |
| L-6/X-413- | L-6/X-414- | L-6/X-415- | L-6/X-416- | L-6/X-417- | L-6/X-418- |

In addition to the combinatorial aspects of this invention, there is also provided an iterative process for rationally evaluating what molecular constraints impart multibinding properties to a class of multimeric macromolecular compounds or ligands targeting a macromolecular receptor. Specifically, this method aspect is directed to a method for identifying multimeric macromolecular ligand compounds possessing multibinding properties which method comprises:

(a) preparing a first collection or iteration of multimeric compounds which is prepared by contacting at least two stoichiometric equivalents of a macromolecular ligand or mixture of ligands which target a receptor with a linker or mixture of linkers wherein said ligand or mixture of ligands comprises at least one reactive functionality and said linker or mixture of linkers comprises at least two functional groups having complementary reactivity to at least one of the reactive functional groups of the ligand wherein said contacting is conducted under conditions wherein the complementary functional groups react to form a covalent linkage between said linker and at least two of said ligands;

(b) assaying said first collection or iteration of multimeric compounds to assess which if any of said multimeric compounds possess multibinding properties;

(c) repeating the process of (a) and (b) above until at least one multimeric compound is found to possess multibinding properties;

(d) evaluating what molecular constraints imparted multibinding properties to the multimeric compound or compounds found in the first iteration recited in (a)-(c) above;

(e) creating a second collection or iteration of multimeric compounds which elaborate upon the particular molecular constraints imparting multibinding properties to the multimeric compound or compounds found in said first iteration;

(f) evaluating what molecular constraints imparted enhanced multibinding properties to the multimeric compound or compounds found in the second collection or iteration recited in (e) above;

(g) optionally repeating steps (e) and (f) to further elaborate upon said molecular constraints.

Preferably, steps (e) and (f) are repeated at least two times and more preferably from 2 to 50 times, even more preferably from 3 to 50 times and still more preferably form 5 to 10 times.

This iterative process can employ collections of multimeric compounds which are prepared either by conventional sequential synthesis (a synthetic process involving a number of steps to provide a single compound which is then repeated with appropriate alteration in use of reagents or process conditions to provide a second compound and wherein the repetition occurs n times to provide for n multimeric compounds) or by combinatorial synthesis to provide a multiplicity of compounds from a single synthetic pathway. In either event, in the first iteration, the ligands and linkers employed and the type and position of the reactive functional groups thereon are selected to provide a diverse collection of multimeric compounds thereby providing maximal information regarding those molecular constraints imparting multibinding properties. Subsequent iterations fine tune the constraints until a final “lead compound” is prepared.

Pharmaceutical Formulations

When employed as pharmaceuticals, the compounds of formula I are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds described herein associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 0.001 to about 1 g, more usually about 1 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of formula I above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The following formulation examples illustrate representative pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | |
|---|---|
| | Quantity |
| Ingredient | (mg/tablet) |

| | | |
|---|---|---|
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 1.0 | mg |
| Talc | 0.5 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount | |
|---|---|---|
| Active Ingredient | 25 | mg |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

A formulation may be prepared as follows:

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, starch, and magnesium stearate are blended, passed through a No. 20 mesh U.S. sieve, and filled into hard gelatin capsules in 425.0 mg quantities.

### Formulation Example 9

A formulation may be prepared as follows:

| Ingredient | Quantity | |
|---|---|---|
| Active Ingredient | 5.0 | mg |
| Corn Oil | 1.0 | mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Other suitable formulations for use in the present invention can be found in Remington's Pharmaceutical Sciences, Mace Publishing Company, Philadelphia, Pa., 17th ed. (1985).

Utility

In one embodiment, the multibinding compounds of this invention are useful in binding to macromolecular structures of cells or microorganisms such as fungi, bacteria, viruses, etc. either in vitro or in vivo. Accordingly, the compounds of this invention can be used to ameliorate pathologic conditions associated with such cells or microorganisms. When bound to the targeted structure component, the ligands of the multibinding compounds modulate or disrupt the biological process/function of cells which, in some cases, can result in targeted cell death. Such modulation or disruption mediates disease conditions associated with these cells.

When used in treating or ameliorating such conditions, the compounds of this invention are typically delivered to a patient in need of such treatment by a pharmaceutical composition comprising a pharmaceutically acceptable diluent and an effective amount of at least one compound of this invention. The amount of compound administered to the patient will vary depending upon what compound and/or composition is being administered, the purpose of the administration, such as prophylaxis or therapy, the state of the patient, the manner of administration, and the like. In therapeutic applications, compositions are administered to a patient already suffering from an infection in an amount sufficient to at least partially arrest further onset of the symptoms of the disease and its complications. Amounts effective for this use will depend on the judgment of the attending clinician depending upon factors such as the degree or severity of the infection in the patient, the age, weight and general condition of the patient, and the like. Such pharmaceutical compositions may contain more than one compound of the present invention.

As noted above, the compounds administered to a patient are in the form of pharmaceutical compositions described above which can be administered by a variety of routes including oral, rectal, transdermal, subcutaneous, intravenous, intramuscular, etc. These compounds are effective as both injectable and oral deliverable pharmaceutical compositions. Such compositions are prepared in a manner well known in the pharmaceutical art and comprise at least one active compound.

The multibinding compounds of this invention can also be administered in the form of pro-drugs, i.e., as derivatives which are converted into a biologically active compound of formula I in vivo. Such pro-drugs will typically include compounds of formula I in which, for example, a carboxylic acid group, a hydroxyl group or a thiol group is converted to a biologically liable group, such as an ester or thioester group which will hydrolyze in vivo to reinstate the respective group.

The multibinding compounds of this invention have further utility as diagnostic agents, research tools, and for various uses in vitro. They are also useful for treating diseases related to animal health, including avian uses, as insecticides, as fungicides (include agricultural fungicides). Additionally, these compounds are also useful as affinity resins in affinity chromatography.

The following synthetic and biological examples are offered to illustrate this invention and are not to be construed in any way as limiting the scope of this invention. Unless otherwise stated, all temperatures are in degrees Celsius.

### EXAMPLES

In the examples below, the following abbreviations have the following meanings. If an abbreviation is not defined, it has its generally accepted meaning.

| | |
|---|---|
| Å | Angstroms |
| cm = | centimeter |
| DCC = | dicyclohexyl carbodiimide |
| DMA = | N, N-dimethylacetamide |
| DMF = | N, N-dimethylformamide |
| DMSO = | dimethylsulfoxide |
| DPPA = | diphenylphosphoryl azide |
| g = | gram |
| HBTU = | 1-hydroxybenzotrizole |
| HPLC = | high performance liquid chromatography |
| Hunig's base = | diisopropylethylamine |
| MFC = | minimum fungicidal concentration |
| mg = | milligram |
| MIC = | minimum inhibitory concentration |
| min = | minute |
| mL = | milliliter |
| mm = | millimeter |
| mmol = | millimol |
| N = | normal |
| PyBOP = | pyridine benzotriazol-1-yloxy-tris(dimethyl- |
| | amino)phosphonium hexafluorophosphate |
| t-BOC = | tert-butyloxycarbonyl |
| TFA = | trifluoroacetic acid |
| THF = | tetrahydrofuran |
| μL = | microliters |
| μm = | microns |

Examples 1-6 are given as representative examples of methods for preparing the linkers.

### Example 1

### Preparation of [C-C] Compounds of Formula I

(1) Preparation of a Compound of Formula (3) in which m is 2 and n is 3

To a solution of tert-butyl N-(2-aminoethyl)carbamate (2.3 g, 14.4 mmol) and N,N-diisopropylethylamine (2.5 mL, 14.3 mmol) in 15 mL methylene chloride at 0° C. was added dropwise glutaryl dichloride (0.6 mL, 4.7 mmol) in 15 mL methylene chloride. The resulting mixture was allowed to warm to room temperature with stirring while adding water (15 mL). The methylene chloride was removed under reduced pressure and more water was added (30 mL). The resulting suspension was filtered and washed sequentially with 10% potassium hydrogen sulfate, water, saturated sodium bicarbonate, and water. The solid was dried under vacuum yielding 1.3 g (3.1 mmol, 66%) of pentanedioic acid bis-[(2-t-butoxycarbonylaminoethyl)amide], a compound of formula (3).

Similarly, varying the composition of m and n, other compounds of formula (3) can be prepared.

(2) Preparation of a Compound of Formula (4) in which m is 2 and n is 3

Pentanedioic acid bis-[(2-t-butoxycarbonylaminoethyl)amide], a compound of formula (3) (1.3 g, 3.1 mmol) was suspended in 15 mL methylene chloride. 15 mL of trifluoroacetic acid was added at room temperature giving (with effervescence) a solution that was stirred for 40 minutes, then evaporated in vacuo. The residue was dissolved in methanol and treated with 3 mL of 4 N hydrogen chloride in dioxane followed by diethyl ether, giving a gum. The liquids were decanted and the gum dried under vacuum yielding 1.0 g (3.4 mmol) of pentanedioic acid bis-[(2-aminoethyl)amide], a compound of formula (4).

Similarly, varying m and n, other compounds of formula (4) can be prepared.

(3) Preparation of a Compound of Formula I

At room temperature, a carboxyl containing ligand (e.g., amphotericin) (2.3 mmol) is dissolved in 36 mL of DMSO. To this solution is added pentanedioic acid bis-[(2-aminoethyl)amide], a compound of formula (4) (1.0 g, 3.4 mmol) suspended in 27 mL DMF followed by addition of N,N-diisopropylethylamine (2.4 mL, 13.8 mmol). The resulting suspension is stirred at room temperature for several hours until it is mostly soluble. Then a solution of PyBOP (1.3 g, 2.5 mmol) and 1-hydroxybenzotriazole (310 mg, 2.3 mmol) in 9 mL DMF is added rapidly dropwise. The mixture is stirred at room temperature for 1 hour and then added dropwise to 600 mL of acetonitrile, giving a precipitate that is filtered, washed with acetonitrile, then diethyl ether, and dried under vacuum. The crude product is purified by reverse phase HPLC (50 minute 2-30% acetonitrile in water containing 0.1% trifluoroacetic acid to yield [C]-pentanedioic acid (2-aminoethyl)amide) ligand and [C-C]-pentanedioic acid bis-[(2-aminoethyl)amide]-bis-(ligand), a compound of Formula I as their respective trifluoroacetic acid salts.

(4) Preparation of Other Compounds of Formula I

Similarly, following the procedures of Example 1, steps 1-3, other compounds of Formula I can be prepared.

### Example 2

### Alternative Preparation of [C-C] Compounds of Formula I

(1) Preparation of a Compound of Formula (7) in which m is 2

At room temperature a carboxyl containing ligand for a macromolecular structure containing a carboxyl group such as amantin (4.7 mmol) is dissolved in 75 mL of DMSO. To this solution is added N,N-diisopropylethylamine (4.1 mL, 23.5 mmol) followed by 9-fluorenylmethyl N-(2-aminoethyl)carbamate hydrochloride (1.8 g, 5.6 mmol). To the resulting solution at room temperature is added rapidly dropwise a solution of PyBOP (2.7 g, 5.2 mmol) and 1-hydroxybenzotriazole (630 mg, 4.7 mmol) in 75 mL 1,3-dimethyl-3,4,5,6-tetrahydro-2(1H)-pyrimidinone. The resulting solution is stirred at room temperature for 2 hours, then poured into 800 mL diethyl ether, giving a gum. The diethyl ether is decanted and the gum is washed with additional diethyl ether to give a compound of formula (7).

(2) Preparation of a Compound of Formula (8) in which m is 2

The gum of formula (7) is then taken up in 40 mL of DMF, to which 10 mL of piperidine is added and the solution left to stand at room temperature for 20 minutes. The solution is then added dropwise to 450 mL of acetonitrile giving a precipitate. Centrifugation is followed by decantation of the acetonitrile and the residue washed twice with 450 mL of acetonitrile, once with 450 mL of diethyl ether and air dried. The residue is taken up in water, acidified to pH<5 with a small amount of 3 N hydrochloric acid and purified by reverse-phase HPLC using a gradient of 2-30% acetonitrile in water containing 0.1% trifluoroacetic acid yielding a compound of formula (8).

(3) Preparation of a Compound of Formula I

Compound (8) (400 mg, 220 mmol) and glutaric acid (10 mg, 76 mmol) are dissolved in 5 mL DMF and N,N-diisopropylethylamine (140 mL, 800 mmol) followed by addition of PyBOP (83 mg, 160 mmol) and 1-hydroxybenzotriazole (10 mg, 74 mmol) in 500 mL DMF. The reaction is stirred for 75 minutes at room temperature then an additional 20 mg of PyBOP is added. 75 minutes later the solution is dripped into 45 mL acetonitrile. The resulting precipitate is collected by centrifugation, washed with ether, air dried and purified by reverse-phase HPLC (50 min 2-30% acetonitrile in water containing 0.1% trifluoroacetic acid, elutes at 33 min) to give a compound of Formula I as its trifluoroacetic salt.

(4) Preparation of Other Compounds of Formula I

Accordingly, following the procedures of Example 2, steps 1-3, other [C-C] compounds of Formula I can be prepared.

### Example 3

### Preparation of a [C-V] Compound of Formula I in which Position C is Substituted

(1) Preparation of a Compound of Formula (22) in which m and n are both 2

To a solution of an amino containing ligand (26.8 mmol) in DMF (2.0 mL) is added a compound of the formula:

Ligand-C(O)NHCH₂CH₂NHC(O)CH₂CH₂C(O)₂Fm

(Fm refers to Fluorenylmethyl ester) (26.8 mmol), followed by PyBOP (20.9 mg, 40.2 mmol), HOBt (5.40 mg, 40.2 mmol), and Hunig's base (23.3 mL, 134 mmol). The reaction solution is stirred for 1 hour and then added dropwise to 20 mL of acetonitrile giving a precipitate, which was collected by centrifugation. The crude precipitate is dried in air, yielding a compound of formula (22). The compound is used in the next step without further purification.

(2) Preparation of a Compound of Formula (23) in which m and n are both 2

The compound of formula (22) is dissolved in 1 mL of DMF, and 100 mL of piperidine is added to the solution. The solution is allowed to stand at room temperature for 30 minutes, following the course of the reaction by mass spectroscopy. The reaction solution is then added dropwise to 20 mL of acetonitrile, giving a precipitate that is collected by centrifugation. The precipitate is purified by reverse phase HPLC on a Ranin C18 Dynamax column (2.5 cm×25 cm, 8 mm particle size), at 10 mL/min flow rate using 0.045% TFA in water as buffer A and 0.045% TFA in acetonitrile as buffer B (HPLC gradient of 2-50% Buffer B over 90 minutes). The desired product is identified by mass spectroscopy using an API 300 electrospray mass spectrometer and afterwards lyophilized to a white powder to afford compound (23) as a white powder.

Preparation of a Compound of Formula I

The compound of formula (23) prepared above (4.80 mmol) is dissolved in 500 mL of DMF. A ligand for a macromolecular structure having a free amino group, such as a ligand of formula (17) (4.80 mmol ) is added to the solution, followed by PyBOP (2.50 mg, 4.8 μmol), HOBt (0.65 mg, 4.80 mmol) and Hunig's base (6.70 mL, 38.4 mmol). The reaction solution is added dropwise to 20 mL of acetonitrile, giving a precipitate that is collected by centrifugation. The precipitate is purified by reverse phase HPLC on a Ranin C18 Dynamax column (2.5 cm×25 cm, 8 mm particle size), at 10 mL/min flow rate using 0.045% TFA in water as buffer A and 0.045% TFA in acetonitrile as buffer B (HPLC gradient of 2-50% B over 90 minutes). The desired product is identified by mass spectroscopy using an API 300 electrospray mass spectrometer.

### Example 4

### Preparation of a [C-N] Compound of Formula I

(1) Preparation of a Compound of Formula (24) in which m is 2

A ligand having both primary and secondary amines such as those found in FIG. 8 (2.60 mmol) is suspended in 40 mL of 1,3-dimethyl-3,4,5,6-tetrahydro-2-(1H)-pyrimidinone and heated to 70° C. for 15 minutes. N-(9-fluorenylmethoxycarbonyl)-aminoacetaldehyde (720 mg, 2.6 mmol) is added and the mixture is heated at 70° C. for one hour. Sodium cyanoborohydride (160 mg, 2.5 mmol) in 2 mL methanol is added and the mixture is heated at 70° C. for 2 hours, then cooled to room temperature. The reaction solution is added dropwise to 20 mL of acetonitrile, giving a precipitate that is collected by centrifugation. The precipitate is purified by reverse phase HPLC on a Ranin C18 Dynamax column (2.5 cm×25 cm, 8 mm particle size), at 10 mL/min flow rate using 0.045% TFA in water as buffer A and 0.045% TFA in acetonitrile as buffer B (HPLC gradient of 10-70% B over 90 minutes), which yielded a compound of formula (24) as its trifluoroacetate salt.

(2) Preparation of a Compound of Formula (25) in which m is 2 and p is 3

The compound of formula (24) obtained above (291 mg, 150 mmol) is dissolved in 3 mL of DMF. 3-(dimethylamino)propylamine (28.3 mL, 225 mmol) is added, followed by the addition of PyBOP (85.8 mg, 165 mmol), HOBt (20.3 mg, 150 mmol) and Hunig's base (65.0 mL, 375 mmol). The reaction solution is stirred for one hour then added 20 mL of acetonitrile is added dropwise to give a precipitate, which was collected by centrifugation. Recovery of this precipitate provides for a compound of formula (25).

(3) Preparation of a Compound of Formula (26) in which m is 2 and p is 3

The compound of formula (25) obtained above is dissolved in 1 mL of DMF, and 100 mL of piperidine is added to the solution. The solution is allowed to stand at room temperature for 30 minutes and the course of the reaction is followed by mass spectroscopy. The reaction solution is added dropwise to 20 mL of acetonitrile, giving a precipitate that is collected by centrifugation. The precipitate is purified by reverse phase HPLC on a Ranin C18 Dynamax column (2.5 cm×25 cm, 8 mm particle size), at 10 mL/min flow rate using 0.045% TFA in water as buffer A and 0.045% TFA in acetonitrile as buffer B (HPLC gradient of 2-50% B over 90 minutes), which yielded the compound of formula (26) as its trifluroacetate salt.

Preparation of a Compound of Formula I

The compound of formula (26) prepared above (3.14 mmol) is dissolved in 500 mL of DMF. A compound of formula (19) (3.14 mmol) is added to the solution, followed by PyBOP (2.44 mg, 4.8 mmol), HOBt(0.65 mg, 4.8 mmol) and Hunig's base (6.7 μL, 38.4 mmol). The reaction solution is added dropwise to 20 mL of acetonitrile, giving a precipitate that is collected by centrifugation. The precipitate is purified by reverse phase HPLC on a Ranin C18 Dynamax column (2.5 cm×25 cm, 8 mm particle size), at 10 mL/min flow rate using 0.045% TFA in water as buffer A and 0.045% TFA in acetonitrile as buffer B (HPLC gradient of 2-50% B over 90 minutes), which yielded a compound of Formula I as its trifluroacetate salt.

### Example 5

### Preparation of an [N-N] Compound of Formula I

(1) Preparation of a Compound of Formula I

The compound of formula (26) prepared above (12.7 mmol) is dissolved in 500 mL of DMF. HO₂C(CH₂)ₙCO₂H (6.34 mmol) is added, followed by PyBOP (8.24 mg, 15.8 mol), HOBt (2.13 mg, 15.8 mmol) and Hunig's base (8.8 ml, 51.0 mmol). The reaction solution is added dropwise to 20 mL of acetonitrile, giving a precipitate that is collected by centrifugation. The precipitate is purified by reverse phase HPLC on a Ranin C18 Dynamax column (2.5 cm×25 cm, 8 mm particle size), at 10 mL/min flow rate using 0.045% TFA in water as buffer A and 0.045% TFA in acetonitrile as buffer B (HPLC gradient of 2-50% B over 90 minutes), which yielded a compound of formula I as its trifluoroacetate salt.

### Example 6

### Preparation of a [C-V] Compound of Formula I

(1) Preparation of a Compound of Formula (27) in which m is 2

The synthesis of compounds 27 and 28 are illustrated in FIG. 43 and 44.

A ligand for a macromolecular structure having a carboxyl group, a primary amino group (NH₂) and a secondary amino group (NHCH₃) (3.2 mmol) is suspended in 40 mL of 1,3-dimethyl-3,4,5,6-tetrahydro-2-(1H)-pyrimidinone and is heated to 70° C. for 15 minutes. 4-Butoxybenzaldehyde (570 mg, 3.2 mmol) is added and the mixture is heated at 70° C. for an additional hour. Sodium cyanoborohydride (241 mg, 3.8 mmol) in 2 mL methanol is added and the mixture is heated to 70° C. for 2 hours and is then cooled to room temperature. The reaction solution is added dropwise to 20 mL of acetonitrile, giving a precipitate that is collected by centrifugation. The precipitate is purified by reverse phase HPLC on Ranin C18 Dynamax column (2.5 cm×25 cm, 8 μm particle size), at 10 mL/min flow rate using 0.045% TFA in water as Buffer A and 0.045% TFA in acetonitrile as Buffer B (HPLC gradient of 10-70% B over 90 minutes), yielding a compound of formula (27) as its trifluoroacetate salt.

(2) Preparation of a Compound of Formula (28) where m is 2

The compound of formula (27) prepared above (45 μmol) is dissolved in 2 mL of DMF. Ethylene diamine (13.4 mg, 22.3 μmol) is added followed by PyBOP (28.0 mg, 54 μmol), HOBt (7.2 mg, 54 μmol) and Hunig's base (63.0 μL, 360 μmol). The reaction solution is added dropwise to 20 mL of acetonitrile, giving a precipitate that is collected by centrifugation. The precipitate is purified by reverse phase HPLC on Ranin C18 Dynamax column (2.5 cm×25 cm, 8 μm particle size), at 10 mL/min flow rate using 0.045% TFA in water as Buffer A and 0.045% TFA in acetonitrile as Buffer B (HPLC gradient of 2-50% B over 90 minutes), yielding a compound of formula (28) as its trifluoroacetate salt.

(3) Preparation of a [C-V] Compound of Formula I in which One V Position is Substituted, wherein m and n are 2 and p is 3

The preparation of the compound of this example is illustrated in FIG. 44.

The compound of formula (28) prepared as above (4.9 μmol) is dissolved in 500 μL of DMF. The compound of formula (19) (4.9 μmol) is added to the solution, followed by PyBOP (3.06 mg, 5.9 μmol), HOBt (0.80 mg, 5.9 μmol) and Hunig's base 6.7 μL, 38.4 μmol). The reaction solution is added dropwise to 20 mL of acetonitrile, giving a precipitate that is collected by centrifugation. The precipitate is purified by reverse phase HPLC on Ranin C18 Dynamax column (2.5 cm×25 cm, 8 μm particle size), at 10 mL/min flow rate using 0.045% TFA in water as Buffer A and 0.045% TFA in acetonitrile as Buffer B (HPLC gradient of 2-50% B over 90 minutes), yielding a compound of Formula I as its trifluoroacetate salt.

From the foregoing description, various modifications and changes in the compositions and methods of this invention will occur to those skilled in the art. All such modifications coming within the scope of the appended claims are intended to be included therein.

What is claimed is:

1. A multibinding compound and salts thereof comprising 2 to 10 macromolecular ligands which may be the same or different and which are covalently attached to a linker or linkers which may be the same or different, each of said ligands comprising a ligand domain capable of binding to a macromolecular structure of a cell with the proviso excluding multimeric compounds having DNA binding as a primary mode of action.

2. A multibinding compound represented by formula I:
(L)ₚ(X)_{q} I
wherein each L is independently selected from macromolecular ligands comprising a ligand domain capable of binding to a macromolecular structure of a cell; each X is independently a linker; p is an integer of from 2 to 10; q is an integer of from 1 to 20 and salts thereof with the proviso excluding multimeric compounds having DNA binding as a primary mode of action.

3. The multibinding compound according to claim 2 wherein q is <p.

4. The multibinding compound according to claim 1 or claim 2 wherein the compound is dimeric.

5. The multibinding compound according to claim 4 wherein the dimeric compound is heterodimeric.

6. The multibinding compound according to claim 1 or claim 2 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

7. The multibinding compound according to claim 6 wherein the linkers comprise linkers of different chain length and/or having different complementary reactive groups.

8. The multibinding compound according to claim 7 wherein the linkers are selected to have different linker lengths ranging from about 2 to 100 Å.

9. The multibinding compound according to claim 8 wherein the linkers are selected to have different linker lengths ranging from about 3 to 40 Å.

10. The multibinding compound according to claim 1 or 2 wherein the linker is represented by the formula::
—X′—Z—(Y′—Z)ₘ—Y″—Z—X′—
in which:
m is an integer of from 0 to 20;
X′ at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;
Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;
Y′ and Y″ at each separate occurrence are selected from the group consisting of: 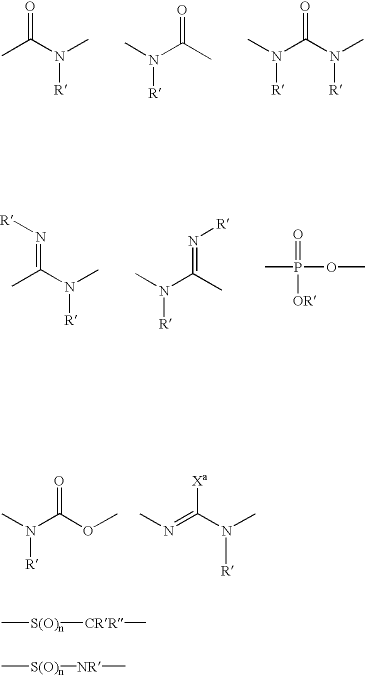
—S—S— or a covalent bond;
in which:
n is 0, 1 or 2; and
R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

11. The multibinding compound according to claim 4 wherein the dimeric compound is homodimeric.

12. The multibinding compound according to claim 11 wherein the two macromolecular ligands are linked to the homodimeric compound at the same point of the ligand.

13. The multibinding compound according to claim 10 wherein the two macromolecular ligands are linked to the homodimeric compound at different points on the ligand.

14. A pharmaceutical composition comprising a pharmaceutically effect excipient and an effective amount of a multibinding compound or a pharmaceutically acceptable salt thereof comprising 2 to 10 ligands which may be the same or different and which are covalently attached to a linker or linkers which may be the same or different, each of said ligands comprising a ligand domain capable of binding to a macromolecular structure of a cell mediating mammalian or avian pathologic conditions thereby inhibiting the pathologic condition with the proviso excluding multimeric compounds having DNA binding as a primary mode of action.

15. A pharmaceutical composition comprising a pharmaceutically acceptable excipient and an effective amount of a multibinding compound represented by formula I:
(L)ₚ(X)_{q} I
wherein each L is independently selected from ligands comprising a ligand domain capable of binding to one or more macromolecular structures of a cell mediating mammalian or avian pathologic conditions; each X is independently a linker; p is an integer of from 2 to 10; q is an integer of from 1 to 20 and pharmaceutically acceptable salts thereof with the proviso excluding multimeric compounds having DNA binding as a primary mode of action.

16. The pharmaceutical composition according to claim 15 wherein q is <p.

17. The pharmaceutical composition according to claim 14 or claim 15 wherein the compound is dimeric.

18. The pharmaceutical composition according to claim 17 wherein the dimeric compound is heterodimeric.

19. The pharmaceutical composition according to claim 14 or claim 15 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

20. The pharmaceutical composition according to claim 19 wherein the linkers comprise linkers of different chain length and/or having different complementary reactive groups.

21. The pharmaceutical composition according to claim 20 wherein the linkers are selected to have different linker lengths ranging from about 2 to 100 Å.

22. The pharmaceutical composition according to claim 21 wherein the linkers are selected to have different linker lengths ranging from about 3 to 40 Å.

23. The pharmaceutical composition according to claim 14 or 15 wherein the linker is represented by the formula::
—X′—Z′(Y′—Z)ₘ—Y″—Z—X′—
in which:
m is an integer of from 0 to 20;
X′ at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;
Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;
Y′ and Y″ at each separate occurrence are selected from the group consisting of: 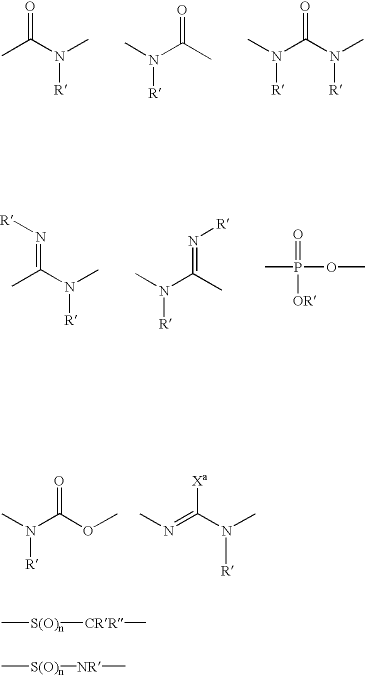
—S—S— or a covalent bond;
in which:
n is 0, 1 or 2; and
R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

24. A method for treating a mammalian or avian pathologic condition mediated by cells comprising macromolecular structures which method comprises administering to said mammal or avian an effective amount of a pharmaceutical composition comprising a pharmaceutically acceptable excipient and a multibinding compound or a pharmaceutically acceptable salt thereof comprising 2 to 10 ligands which may be the same or different and which are covalently attached to a linker or linkers which may be the same or different, each of said ligands comprising a ligand domain capable of binding to a macromolecular structure of the cell(s) mediating mammalian or avian pathologic conditions with the proviso excluding multimeric compounds having DNA binding as a primary mode of action.

25. A method for treating a mammalian or avian pathologic condition mediated by cells comprising macromolecular structures which method comprises administering to said mammal or avian an effective amount of a pharmaceutical composition comprising a pharmaceutically acceptable excipient and a multibinding compound represented by formula I:
(L)ₚ(X)_{q} I
wherein each L is independently selected from ligands comprising a ligand domain capable of binding to one or more macromolecular structures of a cell mediating mammalian pathologic conditions; Each X is independently a linker; p is an integer of from 2 to 10; q is an integer of from 1 to 20 and pharmaceutically acceptable salts thereof with the proviso excluding multimeric compounds having DNA binding as a primary mode of action.

26. A method for modulating the biological processes/functions of a cell which method comprises contacting said cell with a multi-binding compound or a pharmaceutically acceptable salt thereof under conditions sufficient to modulate one or more biological processes/functions of said cell wherein said multi-binding compound comprises 2 to 10 ligands which may be the same or different and which are covalently attached to a linker or linkers which may be the same or different, each of said ligands comprising a ligand domain capable of binding to one or more macromolecular structures of the cells with the proviso excluding multimeric compounds having DNA binding as a primary mode of action.

27. The method according to claim 25 wherein q is <p.

28. The method according to claim 24, claim 25 or claim 26 wherein the compound is dimeric.

29. The method according to claim 28 wherein the dimeric compound is heterodimeric.

30. The method according to claim 24, claim 25 or claim 26 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

31. The method according to claim 30 wherein the linkers comprise linkers of different chain length and/or having different complementary reactive groups.

32. The method according to claim 31 wherein the linkers are selected to have different linker lengths ranging from about 2 to 100 Å.

33. The method according to claim 32 wherein the linkers are selected to have different linker lengths ranging from about 3 to 40 Å.

34. The method compound according to claim 24, claim 25 or claim 26 wherein the linker is represented by the formula::
—X′—Z—(Y′—Z)ₘ—Y″—Z—X′—
in which:
m is an integer of from 0 to 20;
X′ at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;
Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;
Y′ and Y″ at each separate occurrence are selected from the group consisting of: 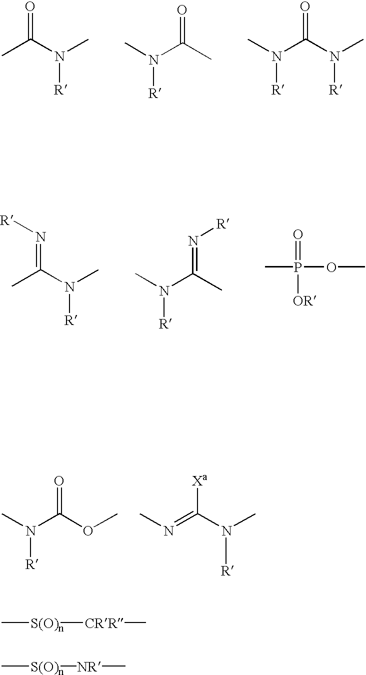
—S—S— or a covalent bond;
in which:
n is 0, 1 or 2; and
R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

35. A method for identifying multimeric macromolecular ligand compounds possessing multibinding properties which method comprises:
(a) identifying a macromolecular ligand or a mixture of ligands wherein each ligand contains at least one reactive functionality;
(b) identifying a library of linkers wherein each linker in said library comprises at least two functional groups having complementary reactivity to at least one of the reactive functional groups of the ligand;
(c) preparing a multimeric macromolecular ligand compound library by combining at least two stoichiometric equivalents of the ligand or mixture of ligands identified in (a) with the library of linkers identified in (b) under conditions wherein the complementary functional groups react to form a covalent linkage between said linker and at least two of said ligands; and
(d) assaying the multimeric macromolecular ligand compounds produced in the library prepared in (c) above to identify multimeric macromolecular ligand compounds possessing multibinding properties.

36. A method for identifying multimeric macromolecular ligand compounds possessing multibinding properties which method comprises:
(a) identifying a library of macromolecular ligands wherein each ligand contains at least one reactive functionality;
(b) identifying a linker or mixture of linkers wherein each linker comprises at least two functional groups having complementary reactivity to at least one of the reactive functional groups of the ligand;
(c) preparing a multimeric macromolecular ligand compound library by combining at least two stoichiometric equivalents of the library of ligands identified in (a) with the linker or mixture of linkers identified in (b) under conditions wherein the complementary functional groups react to form a covalent linkage between said linker and at least two of said ligands; and
(d) assaying the multimeric macromolecular ligand compounds produced in the library prepared in (c) above to identify multimeric macromolecular ligand compounds possessing multibinding properties.

37. The method according to claim 35 or claim 36 wherein the preparation of the multimeric macromolecular ligand compound library is achieved by either the sequential or concurrent combination of the two or more stoichiometric equivalents of the ligands identified in (a) with the linkers identified in (b).

38. The method according to claim 37 wherein the multimeric macromolecular ligand compounds comprising the multimeric ligand compound library are dimeric.

39. The method according to claim 38 wherein the dimeric macromolecular ligand compounds comprising the dimeric ligand compound library are heterodimeric.

40. The method according to claim 39 wherein the heterodimeric macromolecular ligand compound library is prepared by sequential addition of a first and second ligand.

41. The method according to claim 35 or claim 36 wherein, prior to procedure (d), each member of the multimeric macromolecular ligand compound library is isolated from the library.

42. The method according to claim 41 wherein each member of the library is isolated by preparative liquid chromatography mass spectrometry (LCMS).

43. The method according to claim 35 or claim 36 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

44. The method according to claim 43 wherein the linkers comprise linkers of different chain length and/or having different complementary reactive groups.

45. The method according to claim 44 wherein the linkers are selected to have different linker lengths ranging from about 2 to 100 Å.

46. The method according to claim 35 or 36 wherein the ligand or mixture of ligands is selected to have reactive functionality at different sites on said ligands.

47. The method according to claim 46 wherein said reactive functionality is selected from the group consisting of carboxylic acids, carboxylic acid halides, carboxyl esters, amines, halides, pseudohalides, isocyanates, vinyl unsaturation, ketones, aldehydes, thiols, alcohols, anhydrides, boronates, and precursors thereof wherein the reactive functionality on the ligand is selected to be complementary to at least one of the reactive groups on the linker so that a covalent linkage can be formed between the linker and the ligand.

48. The method according to claim 35 or claim 36 wherein the multimeric macromolecular ligand compound library comprises homomeric ligand compounds.

49. The method according to claim 35 or claim 36 wherein the multimeric macromolecular ligand compound library comprises heteromeric ligand compounds.

50. A library of multimeric macromolecular ligand compounds which may possess multivalent properties which library is prepared by the method comprising:
(a) identifying a macromolecular ligand or a mixture of ligands wherein each ligand contains at least one reactive functionality;
(b) identifying a library of linkers wherein each linker in said library comprises at least two functional groups having complementary reactivity to at least one of the reactive functional groups of the ligand; and
(c) preparing a multimeric macromolecular ligand compound library by combining at least two stoichiometric equivalents of the ligand or mixture of ligands identified in (a) with the library of linkers identified in (b) under conditions wherein the complementary functional groups react to form a covalent linkage between said linker and at least two of said ligands.

51. A library of multimeric macromolecular ligand compounds which may possess multivalent properties which library is prepared by the method comprising:
(a) identifying a library of macromolecular ligands wherein each ligand contains at least one reactive functionality;
(b) identifying a linker or mixture of linkers wherein each linker comprises at least two functional groups having complementary reactivity to at least one of the reactive functional groups of the ligand; and
(c) preparing a multimeric macromolecular ligand compound library by combining at least two stoichiometric equivalents of the library of ligands identified in (a) with the linker or mixture of linkers identified in (b) under conditions wherein the complementary functional groups react to form a covalent linkage between said linker and at least two of said ligands.

52. The library according to claim 50 or claim 51 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

53. The library according to claim 52 wherein the linkers comprise linkers of different chain length and/or having different complementary reactive groups.

54. The library according to claim 53 wherein the linkers are selected to have different linker lengths ranging from about 2 to 100 Å.

55. The library according to claim 50 or 51 wherein the macromolecular ligand or mixture of ligands is selected to have reactive functionality at different sites on said ligands.

56. The library according to claim 55 wherein said reactive functionality is selected from the group consisting of carboxylic acids, carboxylic acid halides, carboxyl esters, amines, halides, pseudohalides, isocyanates, vinyl unsaturation, ketones, aldehydes, thiols, alcohols, anhydrides, boronates, and precursors thereof wherein the reactive functionality on the ligand is selected to be complementary to at least one of the reactive groups on the linker so that a covalent linkage can be formed between the linker and the ligand.

57. The library according to claim 50 or claim 51 wherein the multimeric macromolecular ligand compound library comprises homomeric ligand compounds.

58. The library according to claim 50 or claim 51 wherein the multimeric macromolecular ligand compound library comprises heteromeric ligand compounds.

59. An iterative method for identifying multimeric macromolecular ligand compounds possessing multibinding properties which method comprises:
(a) preparing a first collection or iteration of multimeric macromolecular compounds which is prepared by contacting at least two stoichiometric equivalents of a macromolecular ligand or mixture of ligands which target a receptor with a linker or mixture of linkers wherein said ligand or mixture of ligands comprises at least one reactive functionality and said linker or mixture of linkers comprises at least two functional groups having complementary reactivity to at least one of the reactive functional groups of the ligand wherein said contacting is conducted under conditions wherein the complementary functional groups react to form a covalent linkage between said linker and at least two of said ligands;
(b) assaying said first collection or iteration of multimeric macromolecular compounds to assess which if any of said multimeric compounds possess multibinding properties;
(c) repeating the process of (a) and (b) above until at least one multimeric macromolecular compound is found to possess multibinding properties;
(d) evaluating what molecular constraints imparted or are consistent with imparting multibinding properties to the multimeric macromolecular compound or compounds found in the first iteration recited in (a)-(c) above;
(e) creating a second collection or iteration of multimeric macromolecular compounds which elaborates upon the particular molecular constraints imparting multibinding properties to the multimeric compound or compounds found in said first iteration;
(f) evaluating what molecular constraints imparted or are consistent with imparting enhanced multibinding properties to the multimeric macromolecular compound or compounds found in the second collection or iteration recited in (e) above;
(g) optionally repeating steps (e) and (f) to further elaborate upon said molecular constraints.

60. The method according to claim 59 wherein steps (e) and (f) are repeated from 2-50 times.

61. The method according to claim 60 wherein steps (e) and (f) are repeated from 5-50 times.
